(19) United States
(12) Patent Application Publication
(10) Pub. No.: US 2023/0159452 A1
(43) Pub. Date: May 25, 2023

(54) REGIOSELECTIVE OXIDATION OF HETEROCYCLIC ALPHA-AMINO AMIDES

(71) Applicant: Pharmazell GmbH, Raubling (DE)

(72) Inventors: Siegfried R. WALDVOGEL, Gau-Algesheim (DE); Sebastian ARNDT, Mainz (DE); Dominik WEIS, Mainz (DE); Kai DONSBACH, Traunstein (DE); Alexander Matthias NAUTH, Mainz (DE); Till OPATZ, Oberursel (DE)

(21) Appl. No.: 17/996,844

(22) PCT Filed: Apr. 23, 2021
(86) PCT No.: PCT/EP2021/060639

§ 371 Date: Oct. 21, 2022

(30) Foreign Application Priority Data
Apr. 24, 2020 (EP) 20171351.8
May 5, 2020 (EP) 20172908.4
Oct. 2, 2020 (EP) 20199842.4
Dec. 11, 2020 (EP) 20213425.0

(51) Int. Cl. C07D 207/27; C25B 3/05; C25B 11/043; B01J 23/46

(52) U.S. Cl. CPC C07D 207/27; C25B 3/05; C25B 11/043; B01J 23/462

(57) ABSTRACT

The present invention relates to regioselective chemical and electrochemical processes for the preparation of an oxidized heterocyclic alpha-amino amide compounds. By applying specific catalysts or catalyst systems during chemical oxidation or by applying particular electrochemical oxidation conditions the present invention provides access to valuable alpha amino amide compounds, which are oxidized at the heterocyclic amino group by regioselective introduction of either a hydroxyl or a keto group. In a more particular embodiment, the present invention describes a chemical oxidation reaction, which advantageously is applicable in the enantioselective synthesis of valuable oxidized heterocyclic alpha-amino amide compounds, like levetiracetam, brivaracetam or the synthesis of piracetam. Another aspect of the present invention relates to a process for the electrochemical recycling of alkali perhalogenate oxidants as spent during said regioselective oxidation reactions of the invention. Still another aspect of the invention relates to the electrochemical preparation of perhalogenates.

The present invention relates to regioselective chemical and electrochemical processes for the preparation of an oxidized heterocyclic alpha-amino amide compounds. By applying specific catalysts or catalyst systems during chemical oxidation or by applying particular electrochemical oxidation conditions the present invention provides access to valuable alpha amino amide compounds, which are oxidized at the heterocyclic amino group by regioselective introduction of either a hydroxyl or a keto group. In a more particular embodiment, the present invention describes a chemical oxidation reaction, which advantageously is applicable in the enantioselective synthesis of valuable oxidized heterocyclic alpha-amino amide compounds, like levetiracetam, brivaracetam or the synthesis of piracetam. Another aspect of the present invention relates to a process for the electrochemical recycling of alkali perhalogenate oxidants as spent during said regioselective oxidation reactions of the invention. Still another aspect of the invention relates to the electrochemical preparation of perhalogenates.

### BACKGROUND OF THE INVENTION

### a) Regioselective Chemical and Electrochemical Oxidation of Heterocyclic Alpha-Amino Amide Compounds

The group of oxidized heterocyclic alpha-amino amide compounds encompasses valuable pharmaceutically active ingredients, like levetiracetam, brivaracetam and piracetam.

For example, levetiracetam of the formula LIVa

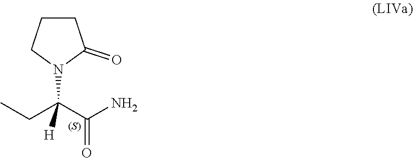

is a valuable drug for treating epilepsy and contains one chiral center. Unfortunately, most of the known synthetic routes need chiral auxiliaries or enantiomerical pure starting materials. (cf. the chemical routes proposed by F. Boschi, P. Camps, M. Comes-Franchini, D. Muñoz-Torrero, A. Ricci, L. Sánchez, Tetrahedron: Asymmetry 2005, 16, 3739-3745; R. Mylavarapu, R. V. Anand, G. C. M. Kondaiah, L. A. Reddy, G. S. Reddy, A. Roy, A. Bhattacharya, K. Mukkanti, R. Bandichhor, Green Chemistry Letters and Reviews 2010, 3, 225-230; and V. Raju, S. Somaiah, S. Sashikanth, E. Laxminarayana, K. Mukkanti, Indian Journal of Chemistry 2014, 53V, 1218-1221.

Different authors report on the regio-selective chemical oxidation of heterocyclic carboxamides (Wei, Y et al, Organic Letters 2011, 13, 7, 1674-1677), N-acyl amines (Pawlik, J. W. et al., J. Am. Chem. Soc. 1981,103, 6755-6757; Kaname, M. et al., Tetrahedron Letters, 2008, 49, 2786-2788), or N-alkyl amides (Minisci, F. et al., J. Org. Chem. 2002, 67, 2671-2676) with different oxidation catalysts. However, none of these documents teaches or suggests the regio-selective chemical oxidation of heterocyclic alpha-amino amides of the present invention.

So far, no synthetic route has been suggested which encompasses, as the final synthetic step, the chemical or electrochemical oxidation of the heterocyclic alpha-amino amide compound, in particular (S)-2-(pyrrolidin-1-yl) butanamide LIa

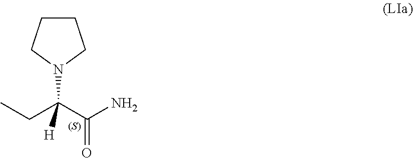

or structurally related alpha amino amide compounds with or without asymmetric carbon atoms in their structure.

A first problem to be solved by the present invention is therefore the provision of a synthetic method for the regioselective oxidation of heterocyclic alpha-amino amides, as for example those of formula LIa and structurally related heterocycles, in alpha position of the heterocyclic amine substituent, as for example the pyrrolidin-1-yl substituent.

A more particular, second problem to be solved by the present invention is the provision of a synthetic method for the regioselective oxidation of such heterocyclic alpha-amino amides comprising an asymmetric carbon atom in alpha position of the carbonyl group in alpha position of the heterocyclic amine substituent, and wherein the oxidation reaction substantially retains the stereochemistry at said asymmetric carbon atom.

### b) Electrochemical Recycling or Preparation of Periodate Oxidant

Electrochemical oxidations of iodate to periodate were reported for lead dioxide anodes (H. H. Willard and R. R. Ralston, Trans. Electrochem. Soc. 1932, 62, 239; C. W. Nam and H. J. Kim, Journal of the Korean Chemical Society 1971, 16, 324; A. Hickling and S. H. Richards, J. Chem. Soc. 1940, 256). Besides, redox mediatory systems were reported that involved the periodate regeneration from iodate at PbO₂ anodes in situ (F. Nawaz Khan, R. Jayakumar, C. N. Pillai, J. Mol. Catal. A: Chemical 2003, 195, 139; U.-St. Bäumer and H. J. Schäfer, Electrochimica Acta 2003, 48, 489; A. Yoshiyama, T. Nonaka, M. M. Baizer, T.-C. Chou, Bull. Chem. Soc. Jpn. 1985, 58, 201; and H. Tanaka, R. Kikuchi, M. Baba, S. Torii, Bull. Chem. Soc. Jpn. 1995, 68, 2989). The oxidation of lithium iodate to periodate was also published at BDD anodes (L. J. J. Janssen, M. H. A. Blijlevens, Electrochimica Acta 2003, 48, 3959; L. J. J. Janssen, NL1013348C2 (2001). Herein, the current efficiency on BDD was compared to platinum and lead electrodes for different iodate and base concentrations. As result, BDD had a similar current efficiency as lead dioxide, but a superior durability. The oxidation of chlorate to perchlorate at BDD anodes was reported in WO2004/055243A and DE10258652A1 (by Lehmann et al.).

However, PbO₂ electrodes disintegrate during electrolysis and generate toxic impurities. Electrode mass losses up to 2.5 g/Ah, or even the generation of lead dioxide particles were reported C. W. Nam and H. J. Kim, Journal of the Korean Chemical Society 1974, 18, 373; Y. Aiya, S, Fujii, K. Sugino, K. Shirai, Journal of the Electrochemical Society 1962, 109, 419; and U.S. Pat. No. 2,830,941A (1958 by Mehltretter)) which is inacceptable for sensitive products.

The iodate oxidation at BDD anodes has only been investigated for lithium iodate, which exhibits the best solubility amongst all alkaline metal iodates. Lithium salts are, however, expensive and yields, analytical data, and scales have not been reported. Hence, no experimental information can be deduced for the oxidation of other iodates. The oxidation of sodium chlorate to perchlorate by Lehmann et al (see above) has been carried out in undivided electrolysis cells that necessitated highly toxic anti-reducing agents. In addition, the properties of sodium perchlorate differ from sodium iodate. Hence, no experimental information can be deduced for the oxidation of iodate to periodate.

A third problem to be solved by the present invention is the provision of a method for the electrochemical preparation or, more particular, the electrochemical recycling of periodates, in particular sodium periodate which avoids the above-mentioned problems reported in the prior art.

### SUMMARY OF THE INVENTION

### a) Regioselective Chemical and Electrochemical Oxidation of Heterocyclic Alpha-Amino Amide Compounds

The above-mentioned first problem of the present invention surprisingly could be solved by the provision of a chemical or electrochemical oxidation reaction, which differs from the known prior art in that it proposes for the first time heterocyclic alpha-amino amide compounds as starting material for a chemical or electrochemical oxidation step resulting in the desired oxidized product carrying a hydroxyl or keto group in alpha-position of its heterocyclic moiety. As the present applicant has observed in a parallel intervention (EP patent application No's: 20171351.8 and 20172908.4) that the above mentioned non-oxidized heterocyclic alpha-amino amide starting materials are surprisingly good accessible, in particular also in stereoisomerical pure form, from the respective heterocyclic nitrile compound, in particular in the form of a mixture of stereoisomers, via an enzyme catalysed step, the present invention also opens the door for a completely new synthesis strategy for oxidized heterocyclic alpha-amino amide compounds. Such oxidized compounds are represented by the general formula II

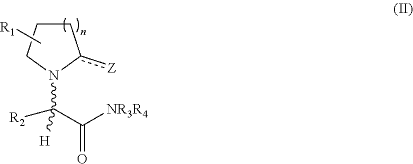

as further defined in the subsequent parts of the description.

Depending on the oxidation reaction conditions of the chemical oxidation reaction, i. p. the type of oxidation catalyst, a product of formula II is obtained, wherein Z represents either a hydroxyl group (OH) or an oxo (═O) group, or a mixture of said two product types is generated.

The above-mentioned second problem of the invention was surprisingly solved by the provision of a chemical oxidation reaction based on a particular type of oxidation catalyst, which allows to run the oxidation reaction under substantial retention of the stereochemistry at said asymmetric carbon atom in alpha position of the carbonyl group of the amide. These oxidation reactions are ruthenium catalyzed and apply a combination of ruthenium dioxide or ruthenium chloride salts each in combination with a periodate comprising oxidant. More particularly, it was surprisingly observed that in the case of such an asymmetric alpha-carbon atom of the substrate the oxidation reaction is performed under essential retention of the stereochemistry. Due to this surprising finding the present invention offers a completely new strategy for the synthesis of the above-mentioned pharmaceutical valuable compounds levetriracetam and brivaracetam, which require a particular stereoisomeric configuration of the carbon atom in alpha position to their amide group.

### b) Electrochemical Recycling or Preparation of Periodate Oxidant

The above-mentioned third problem of the invention was surprisingly solved by the present inventors by the provision an improved method for the electrochemical oxidation of iodate to periodate. The iodate electrolysis of the invention is based on the use of BDD anodes, whereby the conventional use of metal-based electrodes, in particular lead dioxide electrodes, could be avoided. The improved process may be used in the de novo synthesis of periodate oxidants as well as in the recycling of iodate, as obtained during a periodate-based oxidation process.

### DESCRIPTION OF THE FIGURES

FIG. 1: GC Calibration lines for the precursor (S)-1 and for Levetiracetam 3 using caffeine as internal standard.

FIG. 2: LC-PDA calibration lines for NaIO₄ and NaIO₃.

### ABBREVIATIONS

acac Acetylacetonate
BDD Boron Doped Diamond
ee enantiomeric excess
HPLC High performance liquid chromatography
GLC Glass-like carbon
GC Gas chromatography
ox Oxalate
OVAT One Value at a Time
TLC Thin layer chromatography

### General Definitions

### a) General Terms:

For the descriptions herein and the appended claims, the use of “or” means “and/or” unless stated otherwise. Similarly, “comprise”, “comprises”, “comprising”, “include”, “includes”, and “including” are interchangeable and not intended to be limiting.

It is to be further understood that where descriptions of various embodiments use the term “comprising” those skilled in the art would understand that in some specific instances, an embodiment can be alternatively described using language “consisting essentially of” or “consisting of”.

The terms “purified”, “substantially purified”, and “isolated” as used herein refer to the state of being free of other, dissimilar compounds with which a compound of the invention is normally associated in its natural state, so that the “purified”, “substantially purified”, and “isolated” subject comprises at least 0.1%, 0.5%, 1%, 5%, 10%, or 20%, or at least 50% or 75% of the mass, by weight, of a given sample. In one embodiment, these terms refer to the compound of the invention comprising at least 95, 96, 97, 98, 99 or 100%, of the mass, by weight, of a given sample.

The term “about” indicates a potential variation of ±25% of the stated value, in particular ±15%, ±10%, more particularly ±5%, ±2% or ±1%.

The term “essentially” refers to describes narrow range of values of least about 90%, 91%, 92%, 93% 94%, particularly 95%, 96%, 97%, 98%, more particularly 99%, and especially 99.5%, 99.9% or 100% .

The term “substantially” describes a range of values of from about 80 to 100%, such as, for example, 85-99.9%, in particular 90 to 99.9%, more particularly 95 to 99.9%, or 98 to 99.9% and especially 99 to 99.9%.

“Predominantly” refers to a proportion in the range of above 50%, as for example in the range of 51 to 90%, particularly in the range of 55 to 89,9%, more particularly 60 to 85%, like 70 to 80%.

A “main product” in the context of the present invention designates a single compound or a group of at least 2 compounds, like 2, 3, 4, 5 or more, particularly 2 or 3 compounds, which single compound or group of compounds is “predominantly” prepared by a reaction as described herein, and is contained in said reaction in a predominant proportion based on the total amount of the constituents of the product formed by said reaction. Said proportion may be a molar proportion, a weight proportion or, particularly based on chromatographic analytics, an area proportion calculated from the corresponding chromatogram of the reaction products.

A “side product” in the context of the present invention designates a single compound or a group of at least 2 compounds, like 2, 3, 4, 5 or more, particularly 2 or 3 compounds, which single compound or group of compounds is not “predominantly” prepared by a reaction as described herein.

The term “stereoisomers” includes conformational isomers and in particular configuration isomers.

Included in general are, according to the invention, all “stereoisomeric forms” of the compounds described herein, such as “constitutional isomers” and “stereoisomers”.

“Stereoisomeric forms” encompass in particular, “stereoisomers” and mixtures thereof, e.g. configuration isomers, encompassing enantiomers, diastereomers and geometric isomers and mixtures thereof. An enantiomer, or optical isomer is one of two stereoisomers that are mirror images of each other and non-superimposable, as for example (R)- and (S)-enantiomers. Diastereomers contain two or more stereo centers; two diastereomers are not mirror images of each other and are non-superimposable. Geometric isomers are for example E- and Z-isomers. The invention also encompasses any combination of such configuration isomers. If one or more asymmetric centers are present in one molecule, the invention encompasses all combinations of different configurations of these asymmetry centers, e.g. enantiomer pairs and diastereomer pairs.

The term “regiospecificity or “regiospecific” describes the orientation of a reaction that involves a reactant containing at least two possible reaction sites. If such reaction takes place and produces two or more products and one of the products “predominates”, the reaction is said to be “regioselective”. If merely one of the products is produced or “essentially” produced then the reaction is said to be “regiospecific” (i.e. proceed under retention of configuration).

The term “stereo-conserving” reaction describes the influence of a chemical, electrochemical or biochemical reaction on an asymmetrical reactant containing at least one asymmetrical carbon atom. If such reaction takes place and produces a product wherein the stereochemical configuration is not changed at the asymmetrical carbon atom, or is “essentially” not changed at the asymmetrical carbon atom, then the reaction may be classified as “stereo-conserving” or, synonymously, as reaction performed under “stereo retention”.

“Stereoselectivity” describes the ability to produce a particular stereoisomer of a compound in a stereoisomerical pure or enriched form or to specifically or predominantly convert a particular stereoisomer (like enantiomer or diastereomer) in a method as described herein out of a plurality of stereoisomers. More specifically, this means that a product of the invention is enriched with respect to a specific stereoisomer, or a starting material may be depleted with respect to a particular stereoisomer. This may be quantified via the purity % ee-parameter calculated according to the formula:

% ee =[X_{A}−X_{B}]/[X_{A}+X_{B}]*100,

wherein X_{A} and X_{B} represent the molar ratio of the stereoisomers A and B.

The % ee-parameter may also be applied to quantify the so-called “enantiomeric excess” or “stereoisomeric excess” of a particular enantiomer formed or converted or non-converted in a specific reaction. Particular % ee-values are in the range of 50 to 100%, like more particularly 60 to 99.9% even more particularly 70 to 99%, 80 to 98% or 85 to 97%.

The term “essentially stereoisomerical pure” refers to a relative proportion of a particular stereoisomer at least 90%, 91%, 92%, 93% 94%, 95%, 96%, 97%, 98%, 99%, 99.5%, 99.9% or 100% relative to the total amount of stereoisomers of a particular compound.

The terms “selectively converting” or “increasing the selectivity” in general means that a particular stereoisomeric form, as for example the (S)-form, of an asymmetric chemical compound, is converted in a higher proportion or amount (compared on a molar basis) than the corresponding other stereoisomeric form, as for example (R)-form. This is observed either during the entire course of said reaction (i.e. between initiation and termination of the reaction), at a certain point of time of said reaction, or during an “interval” of said reaction. In particular, said selectivity may be observed during an “interval” corresponding 1 to 99%, 2 to 95%, 3 to 90%, 5 to 85%, 10 to 80%, 15 to 75%, 20 to 70%, 25 to 65%, 30 to 60%, or 40 to 50% conversion of the initial amount of the substrate. Said higher proportion or amount may, for example, be expressed in terms of:

a higher maximum yield of an isomer observed during the entire course of the reaction or said interval thereof;

a higher relative amount of an isomer at a defined % degree of conversion value of the substrate; and/or

an identical relative amount of an isomer at a higher % degree of conversion value;

each of which particularly being observed relative to a reference method, said reference method being performed under otherwise identical conditions with known chemical or biochemical means.

Generally also comprised in accordance with the invention are all “isomeric forms” of the compounds described herein, such as constitutional isomers and in particular stereoisomers and mixtures of these, such as, for example, optical isomers, such as (R) and (S)-form, or geometric isomers, such as E- and Z-isomers, and combinations of these. If several centers of asymmetry are present in a molecule, then the invention comprises all combinations of different conformations of these centers of asymmetry, such as, for example, any mixtures of stereoisomeric forms or any mixtures of diastereomers in the case of more than one stereocenter.

“Yield” and/or the “conversion rate” of a reaction according to the invention is determined over a defined period of, for example, 4, 6, 8, 10, 12, 16, 20, 24, 36, or 48 hours, in which the reaction takes place. In particular, the reaction is carried out under precisely defined conditions, for example at “standard conditions” as herein defined.

If the present disclosure refers to features, parameters and ranges thereof of different degree of preference (including general, not explicitly preferred features, parameters and ranges thereof) then, unless otherwise stated, any combination of two or more of such features, parameters and ranges thereof, irrespective of their respective degree of preference, is encompassed by the disclosure of the present description.

### b) Chemical Terms:

The term “lactam derivative” in the context of the present invention in particular refers to chemical compounds which are obtained from a chemical precursor compound comprising a cyclic amino group by an enzymatic or, in particular, chemical oxidation reaction converting said cyclic amino group to a lactam (or intramolecular amide) group.

“Levetiracetam” designates the chemical compound (S)-2-(2-oxopyrrolidin-1-yl)butanamide CAS-Number: 102767-28-2

“Brivaracetam” designates the chemical compound (2S)-2-[(4R)-2-Oxo-4-propylpyrrolidin-1-yl]butanamide; CAS-Number: 357336-20-0

“Piracetam” designates the chemical compound 2-(2-oxopyrrolidin-1-yl)acetamide CAS-Number: 7491-74-9

A “hydrocarbon” group is a chemical group, which essentially is composed of carbon and hydrogen atoms and may be a non-cyclic, linear or branched, saturated or unsaturated moiety, or a cyclic saturated or unsaturated moiety, aromatic or non-aromatic moiety. A hydrocarbon group comprises 1 to 30, 1 to 25, 1 to 20, 1 to 15, or 1 to 10, or 1 to 6, or 1 to 3 carbon atoms in the case of a non-cyclic structure. It comprises 3 to 30, 3 to 25, 3 to 20, 3 to 15, 3 to 10 or in particular 3, 4, 5, 6, or 7 carbon atoms in the case of a cyclic structure. Particularly, it is a non-cyclic, linear or branched, saturated or unsaturated, particularly saturated moiety, comprises 1 to 10, or particularly 1 to 6, or more particularly 1 to 3 carbon atoms

Said hydrocarbon groups may be non-substituted or may carry at least one, like 1, 2, 3, 4, or 5, 2 substituents; particularly it is non-substituted.

Particular examples of such hydrocarbon groups are noncyclic linear or branched alkyl or alkenyl residues as defined below.

An “alkyl” residue represents linear or branched, saturated hydrocarbon residues. The term comprises long chain and short chain alkyl groups. It comprises 1 to 30, 1 to 25, 1 to 20, 1 to 15 or 1 to 10 or 1 to 7, particularly 1 to 6, 1 to 5, or 1 to 4 or more particularly 1 to 3 carbon atoms.

An “alkenyl” residue represents linear or branched, mono- or polyunsaturated hydrocarbon residues. The term comprises long chain and short chain alkenyl groups. It comprises 2 to 30, 2 to 25, 2 to 20, 2 to 15 or 2 to 10 or 2 to 7, particularly 2 to 6, 2 to 5, or more particularly2 to 4 carbon atoms. I may have up to 10, like 1, 2, 3, 4 or 5, particularly 1 or 2, more particularly 1 C═C double bonds.

The term “lower alkyl” or “short chain alkyl” represents saturated, straight-chain or branched hydrocarbon radicals having 1 to 3, 1 to 4, 1 to 5, 1 to 6, or 1 to 7, in particular 1 to 3 carbon atoms. As examples there may be mentioned: methyl, ethyl, n-propyl, 1-methylethyl, n-butyl, 1-methylpropyl, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; and also n-heptyl, and the singly or multiply branched analogs thereof.

“Long-chain alkyl” represents, for example, saturated straight-chain or branched hydrocarbyl radicals having 8 to 30, for example 8 to 20 or 8 to 15, carbon atoms, such as octyl, nonyl, decyl, undecyl, dodecyl, tridecyl, tetradecyl, pentadecyl, hexadecyl, heptadecyl, octadecyl, nonadecyl, eicosyl, hencosyl, docosyl, tricosyl, tetracosyl, pentacosyl, hexacosyl, heptacosyl, octacosyl, nonacosyl, squalyl, constitutional isomers, especially singly or multiply branched isomers thereof.

“Long-chain alkenyl” represents the mono- or polyunsaturated analogues of the above mentioned “long-chain alkyl” groups,

“Short chain alkenyl” (or “ lower alkenyl”) represents mono- or polyunsaturated, especially monounsaturated, straight-chain or branched hydrocarbon radicals having 2 to 4, 2 to 6, or 2 to 7 carbon atoms and one double bond in any position, e.g. C₂-C₆-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The “substituent” of the above mentioned residues contains one hetero atom, like O or N. Particularly the substituents are independently selected from —OH, C═O, or —COOH.

A cyclic saturated or unsaturated moiety as referred to herein particularly refers to monocyclic hydrocarbon groups comprising one optionally substituted, saturated or unsaturated hydrocarbon ring groups (or “carbocyclic” groups). The cycle may comprise 3 to 8, in particular 5 to 7, more particularly 6 ring carbon atoms. As examples of monocyclic residues there may be mentioned “cycloalkyl” groups which are carbocyclic radicals having 3 to 7 ring carbon atoms, such as cyclopropyl, cyclobutyl, cyclopentyl, cyclohexyl, cycloheptyl, and cyclooctyl; and the corresponding “cycloalkenyl” groups. Cycloalkenyl” (or “mono- or polyunsaturated cycloalkyl”) represents, in particular, monocyclic, mono- or polyunsaturated carbocyclic groups having 5 to 8, particularly up to 6, carbon ring members, for example monounsaturated cyclopentenyl, cyclohexenyl, cycloheptenyl and cyclooctenylradicals.

The number of substituents in such cyclic hydrocarbon residues may vary from 1 to 5, in particular 1 or 2 substituents. Suitable substituents of such cyclic residues are selected from lower alkyl, lower alkenyl, or residues containing one heteroatom, like O or N as for example —OH or —COOH. In particular, the substituents are independently selected from —OH, —COOH or methyl.

Unsaturated cyclic groups may contain 1 or more, as for example 1, 2 or 3 C═C bonds and are aromatic, or in particular nonaromatic.

The above-mentioned cyclic groups may also contain at least one, like 1, 2, 3 or 4, preferably 1 or 2 ring heteroatoms, such as O, N or S, particularly N or O.

The term “salt” as used herein, refers in particular to alkali metal salts such as Li, Na and K salts of a compound, alkaline earth metal salts, such as Be, Mg, Ca, Sr and Ba salts of a compound; and ammonium salts, wherein an ammonium salt comprises the NH₄⁺ salt or those ammonium salts in which at least one hydrogen atom can be replaced with a C₁-C₆-alkyl residue. Typical alkyl residues are, in particular, C₁-C₄-alkyl residues, such as methyl, ethyl, n- or i-propyl-, n-, sec- or tert-butyl, and n-pentyl and n-hexyl and the singly or multiply branched analogs thereof.

The term “alkyl esters” of compounds according to the invention are, in particular, lower alkyl esters, for example C₁-C₆-alkyl esters. As non-limiting examples, we may mention methyl, ethyl, n- or i-propyl, n-, sec- or tert-butyl esters, or longer-chain esters, for example n-pentyl and n-hexyl esters and the singly or multiply branched analogs thereof.

Unless stated otherwise, the term “substituent” refers to any residue containing at least one, in particular one hetero atom, like O or N. Particularly such substituents are independently selected from —NH₂, —OH, C═O, or —COOH.

The term “halogenate” unless otherwise stated relates in particular to “metal halogenates” which in turn relates to the metal salts of the respective acids, and encompasses bromates, chlorates, and, in particular, iodates, as well as any optionally existing hydrate form thereof. The alkali metal is K or in particular Na.

The term “iodate” relates to a salt of iodic acid comprising the anion IO₃⁻, as well as any optionally existing hydrate form thereof.

The term “perhalogenates” unless otherwise stated relates in particular to “metal perhalogenates” which in turn relates to the metal salts of the respective perhalogenic acids, and encompasses encompasses perbromates, perchlorates, and, in particular periodates, as well as any optionally existing hydrate form thereof. The alkali metal is K or in particular Na.

The term “periodate” unless otherwise stated relates in particular to “metal periodate” which in turn relates to the metal salts of the various periodic acids. More particularly it relates to “alkali metal periodate, wherein the alkali metal is K or in particular Na. In said periodic acids the corresponding anions are composed of iodine in oxidation state +VII and oxygen. Periodates include i.a. ortho-periodates (IO₆⁵⁻; the metal ortho-periodate thus having the formula M₅IO₆), meta-periodates (IO₄⁻; the metal meta-periodate thus having the formula MIO₄), dimesoperiodates (I₂O₉⁴⁻; the metal dimesoperiodate thus having the formula M₄I₂O₉), mesoperiodates (IO₅³⁻; the metal mesoperiodate thus having the formula M₃IO₅) and para-periodates. Para-periodates are salts of the formula M₃H₂IO₆ and are also known as the corresponding double salt MIO₄*2 MOH. M in the above formulae is a metal equivalent [(Mⁿ⁺)_{1/n}, where n is the charge number]; in case of, for example, an alkali metal periodate M is thus an alkali metal cation; and in case of an earth alkaline metal periodate M is (M²⁺)_{1/2}. In periodates with more than one negative charge, the more than one metal equivalents M can have the same or different meanings. By way of example, in the para-periodates M₃H₂IO₆ or MIO₄*2 MOH all three metal equivalents M can have the same meaning or can be derived from different metals; a situation which can for example occur if the counter cation of the starting material differs from the counter cation present in the base optionally present during anodic oxidation or used during workup of the reaction product.

The term “hypohalogenite” unless otherwise stated relates in particular to “metal hypohalogenites” which in turn relates to the metal salts of the respective hypohalogenic acids, and encompasses hypofluorites, hypobromites, hypoiodites and in particular hypochlorites, as well as any optionally existing hydrate form thereof. The alkali metal is K or in particular Na.

### c) Particular Starting Materials

Particular starting materials of the claimed oxidation reaction are shown below. Middle and right column show particular stereoisomers of the respective starting material depicted in the left column.

TABLE 1
Particular Starting Materials

| | |
|---|---|
| 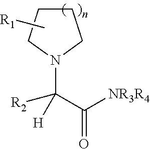 | (I) |
| 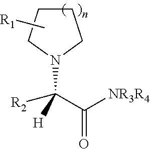 | (Ia) |
| 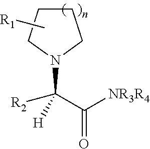 | (Ib) |
| 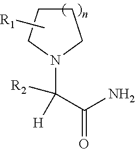 | (XI) |
| 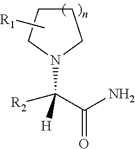 | (XIa) |
| 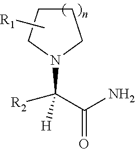 | (XIb) |
| 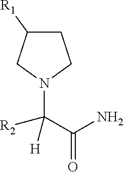 | (XXI) |
| 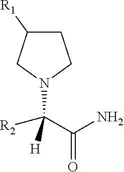 | (XXIa) |
| 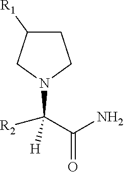 | (XXIb) |
| 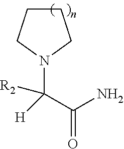 | (XXXI) |
| 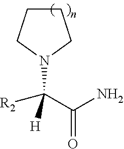 | (XXXIa) |
| 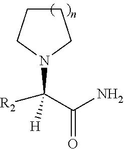 | (XXXIb) |
| 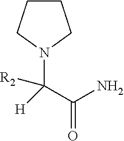 | (XLI) |
| 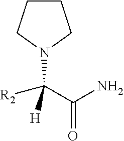 | (XLIa) |
| 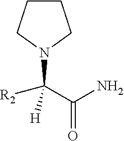 | (XLIb) |
| 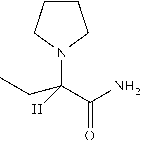 | (LI) |
| 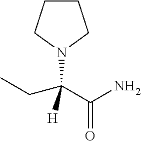 | (LIa) |
| 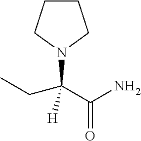 | (LIb) |
| 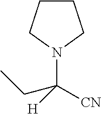 | (IV) |
| 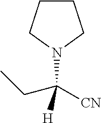 | (IVa) |
| 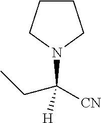 | (IVb) |

### d) Particular Products

Particular products of the claimed oxidation reaction are shown below. Middle and right columns show particular stereoisomers of the respective product depicted in the left column.

TABLE 2
Particular Products

| | |
|---|---|
| 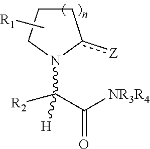 | (II) |
| 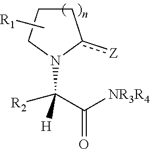 | (IIa) |
| 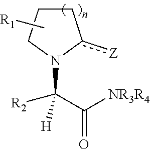 | (IIb) |
| 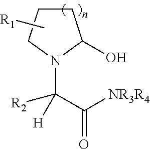 | (III) |
| 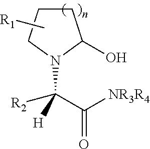 | (IIIa) |
| 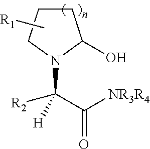 | (IIIb) |
| 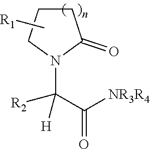 | (IV) |
| 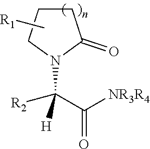 | (IVa) |
| 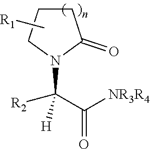 | (IVb) |
| 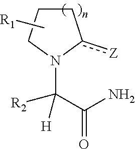 | (XII) |
| 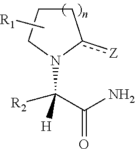 | (XIIa) |
| 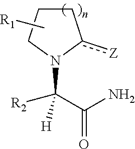 | (XIIb) |
| 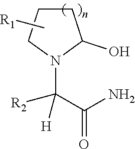 | (XIII) |
| 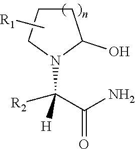 | (XIIIa) |
| 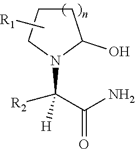 | (XIIIb) |
| 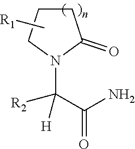 | (XIV) |
| 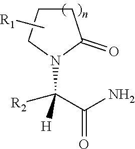 | (XIVa) |
| 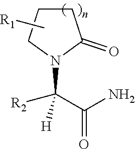 | (XIVb) |
| 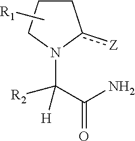 | (XXII) |
| 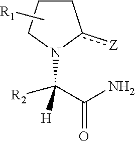 | (XXIIa) |
| 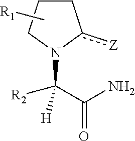 | (XXIIb) |
| 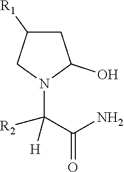 | (XXIII) |
| 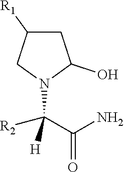 | (XXIIIa) |
| 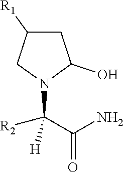 | (XXIIIb) |
| 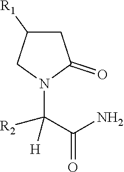 | (XXIV) |
| 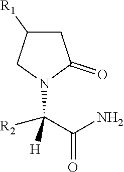 | (XXIVa) |
| 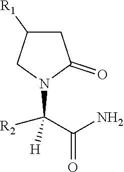 | (XXIVb) |
| 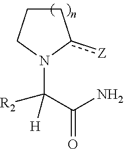 | (XXXII) |
| 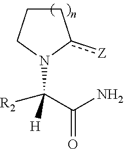 | (XXXIIa) |
| 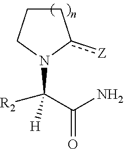 | (XXXIIb) |
| 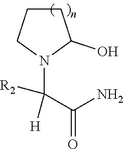 | (XXXIII) |
| 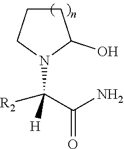 | (XXXIIIa) |
| 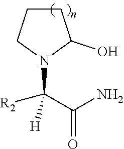 | (XXXIIIb) |
| 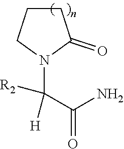 | (XXXIV) |
| 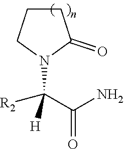 | (XXXIVa) |
| 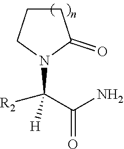 | (XXXIVb) |
| 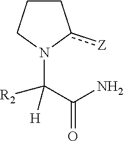 | (XLII) |
| 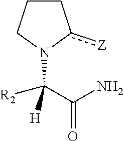 | (XLIIa) |
| 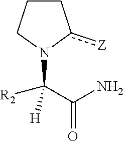 | (XLIIb) |
| 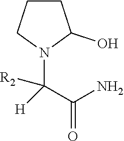 | (XLIII) |
| 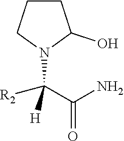 | (XLIIIa) |
| 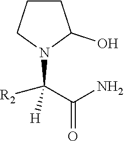 | (XLIIb) |
| 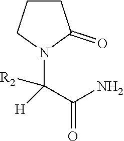 | (XLIV) |
| 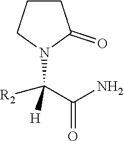 | (XLIVa) |
| 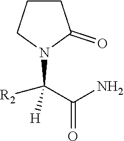 | (XLIVb) |
| 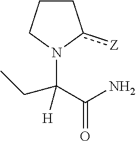 | (LII) |
| 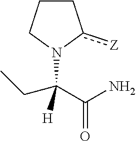 | (LIIa) |
| 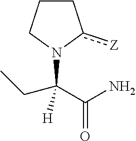 | (LIIb) |
| 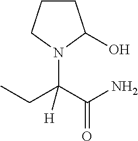 | (LIII) |
| 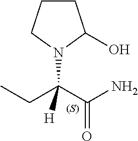 | (LIIIa) |
| 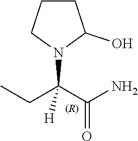 | (LIIIb) |
| 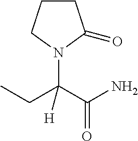 | (LIV) |
| 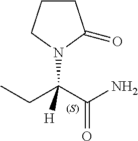 Levetiracetam | (LIVa) |
| 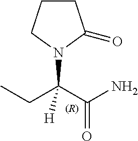 | (LIVb) |
| 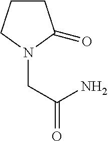 Piracetam | (LV) |
| 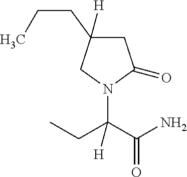 | (LVI) |
| 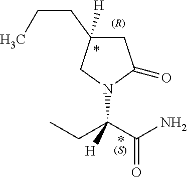 Brivaracetam | (LVIa) |
| 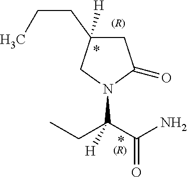 | (LVIb) |

If not stated otherwise the generic parameters of the chemical formulae given in the above tables have the following meanings:

the group Z is bound to the heterocyclic ring via a single or a double bond and is selected from —OH and ═O;

n is 0 or an integer of 1 to 4;

R₁ and R₂ independently of each other represent H or a hydrocarbon group

R₃ and R₄ independently of each other represent H, a straight chain or branched, saturated or non-saturated hydrocarbon group having 1 to 6 carbon atoms; or form, together with the nitrogen atom to which they are bound, a saturated or non-saturated, non-aromatic or aromatic, optionally substituted, in particular non-substituted, heterocyclic 4- to 7-membered ring group carrying one or more ring heteroatoms.

### Special Embodiments of the Invention

The present invention relates to the following particular embodiments.

### a) Regioselective Chemical and Electrochemical Oxidation of Heterocyclic Alpha-Amino Amide Compounds

A first aspect of the invention relates to novel chemical or electrochemical regio-selective oxidation processes of the preparation of certain oxidized heterocyclic alpha-amino amide compounds
1. A first embodiment relates to a process for the preparation of an oxidized heterocyclic alpha-amino amide compound of the general formula (II)

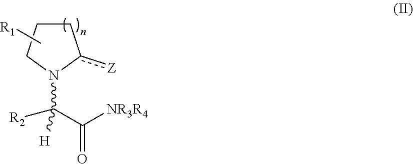

wherein
   the group Z is bound to the heterocyclic ring via a single or a double bond and is selected from —OH and ═O; in particular Z is ═O;
   n is 0 or an integer of 1 to 4; in particular 1 or 2, more particularly 1;
   R₁ and R₂ independently of each other represent H or a hydrocarbon group, in particular a straight-chain or branched, saturated or non-saturated hydrocarbon group having 1 to 6 carbon atoms;
   in particular R₁ and R₂ independently of each other represent H or Cᵢ-C₆ alkyl or C₁-C₃ alkyl, more particularly R₁ and R₂ independently of each other represent H or C₁-C₃ alkyl, like in particular methyl;
   even more particularly R₁ is H and R₂ is ethyl; or R₁ is n-propyl and R₂ is ethyl; or R₁ and R₂ is H;
   R₃ and R₄ independently of each other represent H, a straight-chain or branched, saturated or non-saturated hydrocarbon group having 1 to 6 carbon atoms; or form, together with the nitrogen atom to which they are bound, a saturated or non-saturated, non-aromatic or aromatic, heterocyclic, optionally substituted, in particular non-substituted 4- to 7-membered ring group carrying one or more ring heteroatoms, as for example S, O or N;
   in particular R₃ and R₄ independently of each other represent H or C1-C6 alkyl or C₁-C₃ alkyl, more particularly H or C₁-C₃ alkyl, like in particular methyl; or form, together with the nitrogen atom to which they are bound, a saturated or non-saturated, non-aromatic or aromatic, optionally substituted, in particular non-substituted heterocyclic 5- or 6--membered ring group carrying one or more ring heteroatoms selected from O and N;
   more particularly, R₃ and R₄ independently of each other represent H or C₁-C₃ alkyl, like in particular methyl; or form, together with the nitrogen atom to which they are bound, a saturated or non-saturated, non-aromatic or aromatic, optionally substituted, in particular non-substituted heterocyclic 5- or 6-membered ring group;
   even more particularly R₃ and R₄ represent H;
optionally in essentially stereoisomerical pure form or as a mixture of stereoisomers; like in particular in a proportion of at least 90%, 91%, 92%, 93% 94%, more particularly 95%, 96%, 97%, 98%, 99%, 99.5%, 99.9% or 100% relative to the total amount of stereoisomers of a compound of formula II, in particular in case R₂ is different from H,
   which process comprises
1) either contacting a non-oxidized heterocyclic alpha-amino amide compound of the general formula I

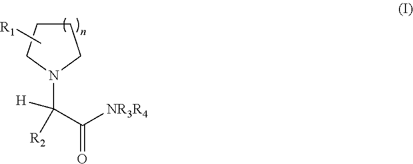

wherein
   Z, n and R₁ to R₄ have the same meanings as defined above,
optionally in essentially stereoisomerical pure form or as a mixture of stereoisomers;
with an oxidation catalyst capable of oxidizing the heterocyclic alpha-amino group in a compound of formula (I), in particular capable of introducing a keto- or hydroxyl group into the heterocyclic amino residue at its alpha-methylene group of a compound of formula (I),
in particular with a heterogeneous or homogenous oxidation catalyst, more particularly a homogeneous catalyst, capable of oxidizing the heterocyclic alpha-amino group in a compound of formula (I) under substantial retention of the stereochemistry at the asymmetric carbon atom in cc-position to the amide group;
or
performing an electrochemical, anodic oxidation of a compound of formula I as defined above;
and
2) optionally isolating at least one compound of formula II.
Such process may be performed batch wise, semi-batch wise or continuously.
More particularly, the process of embodiment 1 encompasses the preparation of an oxidized heterocyclic alpha-amino amide compound of the general formula XII,

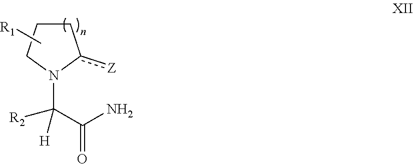

wherein Z, n, R₁ and R₂ have the same meanings as defined above, optionally in essentially stereoisomerical pure form or as a mixture of stereoisomers;
or of the general formula XXII,

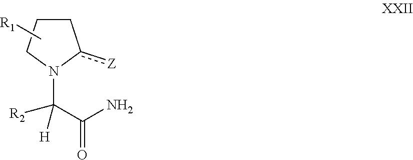

wherein Z and n have the same meanings as defined above, optionally in essentially stereoisomerical pure form or as a mixture of stereoisomers;
or of the general formula XXXII,

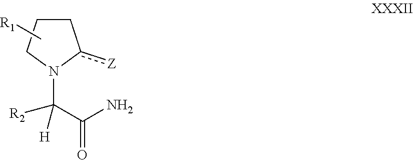

wherein n and R₂ have the same meanings as defined above, optionally in essentially stereoisomerical pure form or as a mixture of stereoisomers;
and more particularly of the general formula XLII,

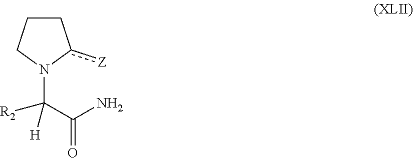

wherein R₂ has the same meanings as defined above, optionally in essentially stereoisomerical pure form or as a mixture of stereoisomers;
and most particularly of the formula LII

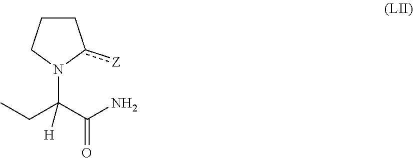

optionally in essentially stereoisomerical pure form or as a mixture of stereoisomers.

The above-mentioned process may result in the formation of an oxidation product consisting essentially of a product wherein the group Z is bound to the heterocyclic ring via a single bond and represents a —OH group, or consisting essentially of a product wherein the group Z is bound via a double bond and represents a ═O group; or alternatively the oxidation product may consist essentially of a mixture of both in any relative ratio and proportion.

For example, a product of the general formula II may be selected from a product of the general formula III or IV or combinations thereof.

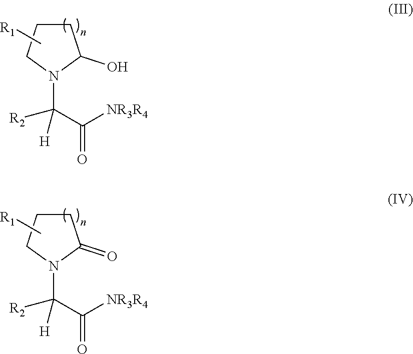

wherein Z, n and R₁ to R₄ have the same meanings as defined above, each compound optionally in essentially stereoisomerical pure form or as a mixture of stereoisomers.

For example, a product of the general formula XII may be selected from a product of the general formula XIII or XIV or combinations thereof.

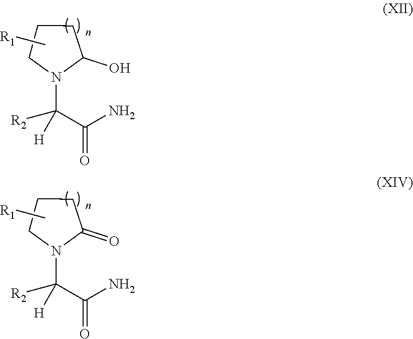

wherein Z, n, R₁ and R₂ have the same meanings as defined above, each compound optionally in essentially stereoisomerical pure form or as a mixture of stereoisomers.

For example, a product of the general formula XXXII may be selected from a product of the general formula XXXIII or XXIV or combinations thereof.

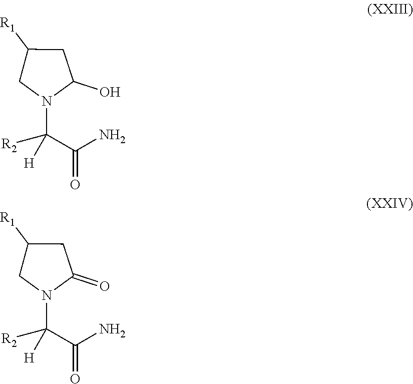

wherein R₁ and R₂ have the same meanings as defined above, each compound optionally in essentially stereoisomerical pure form or as a mixture of stereoisomers.

For example, a product of the general formula XXXII may be selected from a product of the general formula XXXIII or XXXIV or combinations thereof.

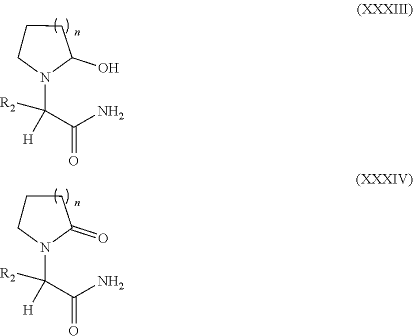

wherein n and R₂ have the same meanings as defined above, each compound optionally in essentially stereoisomerical pure form or as a mixture of stereoisomers.

For example, a product of the general formula XLII may be selected from a product of the general formula XLIII or XLIV or combinations thereof.

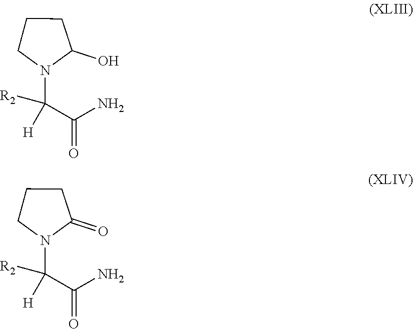

wherein R₂ has the same meanings as defined above, each compound optionally in essentially stereoisomerical pure form or as a mixture of stereoisomers.

For example, a product of the general formula LII may be selected from a product of the general formula LIII or LIV or combinations thereof.

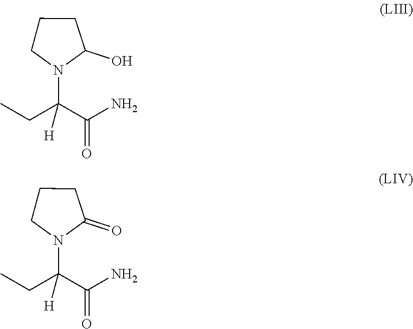

each compound optionally in essentially stereoisomerical pure form or as a mixture of stereoisomers.

More particularly, the process of embodiment 1 encompasses the regiospecific oxidation of a heterocyclic alpha-amino amide compound of the general formula XI.

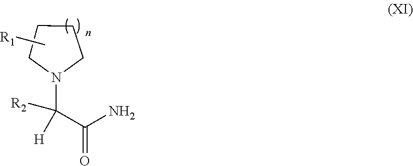

wherein n and R₁ and R₂ have the same meanings as defined above, optionally in essentially stereoisomerical pure form or as a mixture of stereoisomers;

or of the general formula XXI

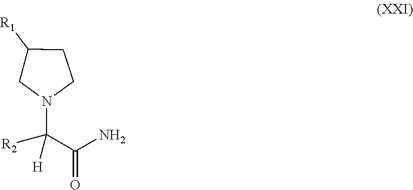

wherein R₁ and R₂ have the same meanings as defined above, optionally in essentially stereoisomerical pure form or as a mixture of stereoisomers;

or of the general formula XXXI

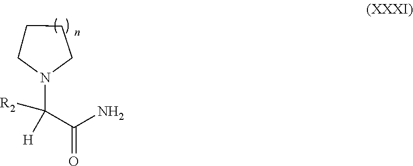

wherein n and R₂ have the same meanings as defined above, optionally in essentially stereoisomerical pure form or as a mixture of stereoisomers;

or of the general formula XLI

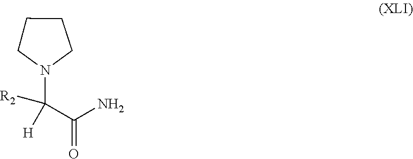

wherein R₂ has the same meanings as defined above, optionally in essentially stereoisomerical pure form or as a mixture of stereoisomers;

or of the general formula LI

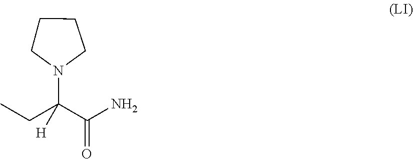

optionally in essentially stereoisomerical pure form or as a mixture of stereoisomers.
2. The process of embodiment 1, wherein the oxidation catalyst is selected from:
   a) a combination of an optionally immobilized inorganic ruthenium salt, in particular a ruthenium (+III), (+IV), (+V), or (+VI) salt, more particularly a ruthenium (+III) or (+IV) salt, and at least one oxidant capable of in situ oxidizing the ruthenium cation, in particular the ruthenium (+III), (+IV), (+V), or (+VI) cation, more particularly the ruthenium (+III) or (+IV) cation, in particular to ruthenium (+VIII), and optionally in the presence of a mono- or polyvalent metal binding ligand, as for example sodium oxalate (ox) or acetylacetonate (acac);
      or
      a combination of an optionally immobilized inorganic osmium salt, in particular an osmium (+III), (+IV), (+V), or (+VI) salt, more particularly an osmium (+III) or (+IV) salt, and at least one oxidant capable of in situ oxidizing the osmium cation, in particular the osmium (+III), (+IV), (+V), or (+VI) cation, more particularly the osmium (+III) or (+IV) cation, in particular to osmium (+VIII), and optionally in the presence of a mono- or polyvalent metal binding ligand, as for example sodium oxalate (ox) or acetylacetonate (acac);
      particularly a combination of an optionally immobilized inorganic ruthenium (+III) or (+IV) salt, and at least one oxidant capable of in situ oxidizing the ruthenium (+III) or (+IV) cation to ruthenium (+VIII), and optionally in the presence of a mono- or polyvalent metal binding ligand, as for example sodium oxalate (ox) or acetylacetonate (acac);
      more particularly a combination of an optionally immobilized inorganic ruthenium (+IV) or (+IV) salt, and at least one oxidant capable of in situ oxidizing the ruthenium (+IV) or (+IV) cation to ruthenium (+VIII), and in the presence of a mono- or polyvalent metal binding ligand, as in particular sodium oxalate (ox) or acetylacetonate (acac);
      Such catalyst systems are suited to catalyze the regio-specific and stereo-conserving (stereo-retentive) oxidation reaction of compounds of the general formula I, in particular of the general formulae XI, XXI, XXXI, and more particularly of the formulae XLI or LI.
      Therefore, if for example a stereoisomer of the formula Ia is applied as substrate, the oxidation reaction produces a product of the formula IIa, or more particularly, of the formula IIIa, or IVa or combinations of IIIa and IVa. If, for example, a stereoisomer of the formula Ib is applied, the oxidation reaction produces a product of the formula IIb, or more particularly, of the formula IIIb or IVb or combinations of IIIb and IVb.
      In analogy, the same is observed if a particular stereoisomer of substrates of the formulae XI, XXI, XXXI, XLI or LI is applied.
   a) a combination of an inorganic iron (+II) or (+III) salt and at least one oxidant capable of in situ oxidizing the iron (+II) or (+III) cation, in particular to iron (+IV), (+V) or (+VI), and optionally in the presence of a mono- or polyvalent metal ligand, as for example sodium oxalate oxalate (ox) or acetylacetonate (acac)
      in particular a combination of an inorganic iron (+II) or (+III) salt and at least one oxidant capable of in situ oxidizing the iron (+II) or (+III) cation to iron (+IV), (+V) or (+VI), and in the presence of a mono- or polyvalent metal ligand, as in particular sodium oxalate oxalate (ox) or acetylacetonate (acac).
      Such catalyst systems are suited to catalyze the regio-specific and optionally stereo-conserving (stereoretentive) oxidation reaction of compounds of the general formula I, in particular of the general formulae XI, XXI, XXXI, and more particularly of the formulae XLI or LI in analogy as described above for the catalyst of section a).
   b) I₂/NaHCO₃
      Such catalyst systems are suited to catalyze the regio-specific and optionally stereo-conserving (stereoretentive) oxidation reaction of compounds of the general formula I, in particular of the general formulae XI, XXI, XXXI, and more particularly of the formulae XLI or LI.
   c) Au-coated Al₂O₃ in the presence of molecular oxygen
      Such catalyst systems are suited to catalyze the regio-specific oxidation reaction of compounds of the general formula I, in particular of the general formulae XI, XXI, XXXI, and more particularly of the formulae XLI or LI.
   d) combinations thereof.
3. The process of embodiment 2, wherein the inorganic ruthenium (+III) or (+IV) salt is selected from RuCl₃, RuO₂ and the respective hydrates thereof (also designated herein RuCl₃.xH₂O, RuO₂.xH₂O) where the stoichiometry x for water is 0.3 or higher, as for example x is about 1, like for example in the rage of about 0.7 to 1.4, or even up to 3 or higher) and wherein the oxidant is selected from
   a) alkali perhalogenates, and their hydrates
   b) alkali hypohalogenites, and their hydrates
   c) Oxone (MPS, potassium monopersulfate)
   d) tert-butyl hydroperoxide (T-HYDRO), optionally in combination with KOtBu
   e) HIO₄
   f) KBrO₃
   g) hypervalent iodide compounds, e.g. DIB (diacetoxyiodobenzene), and
   h) combinations thereof, as for example combinations of a) and b).
   In a particular embodiment the process is performed with a Ruthenium-based oxidation catalyst comprising oxidant a) or b), in particular a).
4. The process of embodiment 2, wherein the inorganic iron (+II) or (+III) salt is selected from FeCl₂, FeCl₃, FeSO₄ and the respective hydrates, and wherein the oxidant is selected from
   a) hydrogen peroxide
   b) T-HYDRO
   c) PhCO₃tBu and
   d) combinations thereof
      Such catalyst systems are suited to catalyze the regio-specific and optionally stereo-conserving oxidation reaction of compounds of the general formula I, in particular of the general formulae XI, XXI, XXXI, and more particularly of the formulae XLI or LI.
5. The process of embodiment 3, wherein the oxidant is selected from
   a) alkali metal periodates, wherein the periodate is a para-periodate, meta-periodate, ortho-periodate or a mixture of two or three of these periodates, and is in particular a para-periodate, a meta-periodate or a mixture of a para-periodate and a metaperiodate, the double salts of such alkali metal periodates, e.g. NaIO₄*2NaOH; and wherein the alkali metal is sodium or potassium and in particular sodium;
   b) alkali metal hypohalogenites, in particular hypochlorites, more particularly NaOCl, and the hydrates thereof, in particular NaOCl*5H₂O;
   c) mixtures of a) and b); or
   d) mixtures of a) and at least one co-oxidant selected from hydrogen peroxide; Oxone (MPS, potassium monopersulfate); T-HYDRO, optionally in combination with KOtBu; I₂/NaHCO3; HIO₄, KBrO₃ and hypervalent iodide compounds, e.g. DIB (diacetoxyiodobenzene).
      Particular examples of oxidation catalysts are selected from
      RuO₂/NaIO₄
      RuO₂*xH₂O/NaIO₄
      RuCl₃*xH₂O/NaIO₄
      RuCl₃*xH₂O/NaOCl*5H₂O and
      RuCl₃*xH₂O/NaIO₄/NaOCl*5H₂O
      wherein x is at least 0.3, as for example x is about 1, like for example in the rage of about 0.7 to 1.4;
      each optionally in combination with a mono- or polyvalent metal ligand, as for example sodium oxalate.
      Further particular examples of oxidation catalysts are selected from
      RuO₂/NaIO₄
      RuO₂*H₂O/NaIO₄
      RuCl₃*H₂O/NaIO₄
      RuCl₃*H₂O/NaOCl*5H₂O and
      RuCl₃*H₂O/NaIO₄/NaOCl*5H₂O
      each optionally in combination with a mono- or polyvalent metal ligand, as for example sodium oxalate.
      NaIO₄ in the above examples is in particular sodium meta-periodate.
      A combination with said mono- or polyvalent metal ligand represents a more particular embodiment, and the following catalyst systems are more particular examples:
      RuO₂/NaIO₄/sodium oxalate
      RuO₂*xH₂O/NaIO₄/sodium oxalate
      RuCl₃*xH₂O/NaIO₄/sodium oxalate
      RuCl₃*xH₂O/NaOCl*5H₂O/sodium oxalate and
      RuCl₃*xH₂O/NaIO₄/NaOCl*5H₂O/sodium oxalate
      wherein x is at least 0.3, as for example x is about 1, like for example in the rage of about 0.7 to 1.4;
      or
      RuO₂/NaIO₄/sodium oxalate
      RuO₂*H₂O/NaIO₄/sodium oxalate
      RuCl₃*H₂O/NaIO₄/sodium oxalate
      RuCl₃*H₂O/NaOCl*5H₂O/sodium oxalate and
      RuCl₃*H₂O/NaIO₄/NaOCl*5H₂O/sodium oxalate
      NaIO₄ in the above examples is in particular sodium meta-periodate.
      Such particular catalyst systems are suited to catalyze the regio-specific and stereo-conserving oxidation reaction of compounds of the general formula I, in particular of the general formulae XI, XXI, XXXI, and more particularly of the formulae XLI or LI.
6. The process of one of the preceding embodiments wherein the reaction product comprises as oxidation product a compound of the above general formula II or a mixture of at least two compounds of general formula II.
7. The process of embodiments 6, wherein the reaction product comprises a compound of the formula III or IV

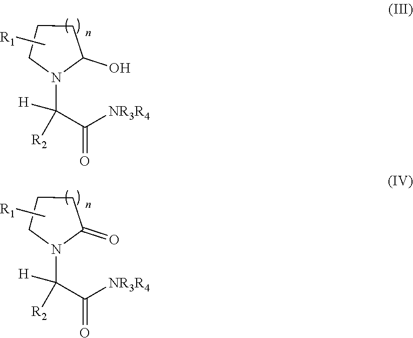

wherein n and R₁ to R₄ have the same meanings as defined above,
each optionally in essentially stereoisomerical pure form or as a mixture of stereoisomers, or a mixture of at least two of said compounds.
8. The process of anyone of the preceding embodiments, wherein the reaction product comprises a compound of the formula III or IV in stereoisomerical essentially pure or enriched form, or as a mixture of at least two stereoisomers.
9. The process of one of the preceding embodiments, wherein said non-oxidized heterocyclic alpha-amino amide compound of the general formula I is applied as a mixture of stereoisomers or in stereoisomerical essentially pure or enriched form.
10. The process of embodiment 9, wherein the oxidation reaction is performed under essential retention of the stereochemical configuration, in particular at the alpha-carbon atom.
11. The process of one of the preceding embodiments, wherein the reaction product comprises one of the following stereoisomer of a compound of formula IV

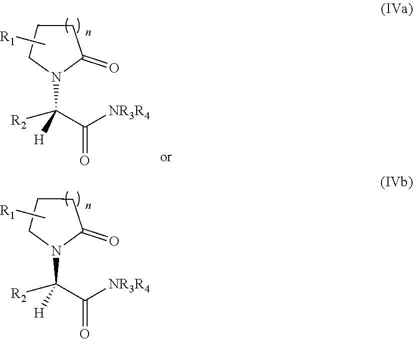

wherein n and R₁ to R₄ have the same meanings as defined above,
or a mixture thereof.
12. The process of embodiment 11, wherein the reaction product comprises a stereoisomer of formula LIVa or LIVb

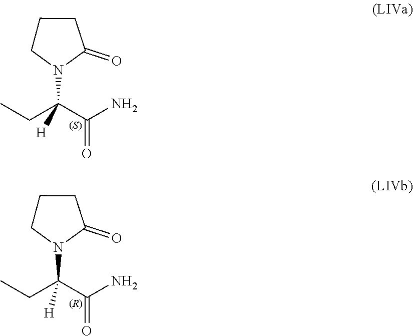

or a mixture thereof, in particular compound LIVa, in essentially stereoisomerical pure form.
13. The process of embodiment 11, wherein the reaction product comprises a stereoisomer of formula LVIa or LVIb

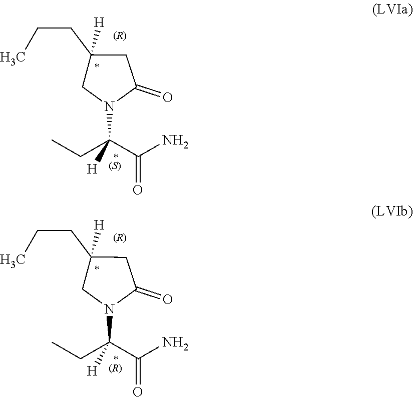

or a mixture of stereoisomers (diastereoisomers) thereof, in particular compound LIVa, in essentially stereoisomerical pure form.
14. The process of anyone of the embodiments 1 to 7 wherein the reaction product comprises a compound of formula XX

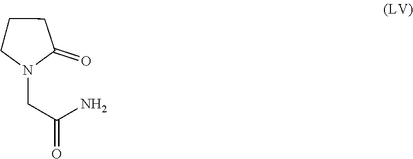

15. The process of anyone of the embodiments 1 to 14, wherein said oxidation is performed by reacting an aqueous or aqueous-organic solution of said compound of formula I at a temperature in the range of 0 to 30° C. with the oxidation catalyst.
16. The process of anyone of the embodiments 2 to 15, wherein the oxidation reaction is performed by reacting said compound of formula I with a catalytic amount of said of said oxidation catalyst, in particular said inorganic ruthenium salt, more particularly said ruthenium (+III) or (+IV) salt and the oxidant, wherein the initial molar ratio of compound of formula I and oxidant is in the range of 1:1 to 1:5, in particular 1:1.5 to 1:3.
17. The process of anyone of the embodiments 2 to 15, wherein the mono- or polyvalent metal ligand is added to the reaction mixture, so that the molar ratio of the oxidation catalyst, in particular said inorganic ruthenium salt, more particularly said ruthenium (+III) or (+IV) salt to ligand is in the range of 1:1 to 1:5, in particular 1:1.5 to 1:2.5.
18. The use of a catalyst or catalyst combination as defined in anyone of the embodiments 2 to 5 in a method for oxidation of heterocyclic alpha-amino amide compounds, carrying a cyclic amino substituent at an asymmetric carbon atom in alpha position the carbonyl C-atom of the amide group.
19. The process of anyone of the embodiments 1 to 18, wherein the chemical oxidation is performed under at least one of the following conditions:
   a) Reaction in an aqueous or aqueous-organic solvent, in particular aqueous solvents, like water, aqueous acid, like diluted sulfuric acid, or aqueous base like diluted sodium hydroxide, or a mixture of one of said aqueous solvents, and at least one organic co-solvent, in particular selected from ethyl acetate, methyl nitrile, acetone, dichloromethane, chloroform, N-methyl-2-pyrrolidone (NMP), dimethylformamide (DMF), dimethyl acetamide (DMAc), Cyrene, Tamisolve, N-ethyl pyrrolidone (NEP), DMSO and DME's.
      Specifically mentioned are solvents selected from water; binary mixtures of water with dimethylformamide, acetonitrile or acetone; as well as ternary mixtures of water/acetone/NMP ; aqueous NaOH/acetone/NMP as well as aqueous sulfuric acid/MeCN/NMP, biphasic solvent systems with water and immiscible ethyl acetate, tetrahydrofurane, diethylether, tert-butylmethylether, cyclo- or n-hexane, or their corresponding homologues.
   b) Said compound of formula I dissolved or suspended in said solvent in an initial concentration in the range of 0.001 to 1 M, in particular from 0.01 to 0.05 M.
   c) Reaction at a temperature in the range of 0 to 60° C., in particular 0 to 30° C.; or at a temperature in the range of −20 to 80° C., or −10 to 60° C., in particular −5 to 20° C.
   According to a very particular embodiment the following combination is applied:
      a hydrated RuCl₃ or RuO₂ pre-catalyst in combination with sodium meta-periodate
      mixtures of aqueous and organic solvent such as dimethylformamide, acetonitrile, or acetone (biphasic mixtures);
      a neutral pH of the reaction medium
      an excess of periodate (in particular ≥2.6 eq.) and
      a low temperature (about 0° C.)
20. The process of embodiment 1 wherein the electrochemical oxidation of a compound of formula I is performed under at least one of the following conditions, in order to obtain products of formula II, in particular of the formula XII, XXII, XXXII, more particularly XLII or LII, wherein Z is ═O or —OH or a mixture of such products:
   a) anode material selected from glass-like carbon (GLC), graphite, boron-doped diamond (BDD), carbon fiber, Ni, Pt, Cu, and Au, or, less preferably stainless steel, Nb, W or Mo;
   b) aqueous solution of at least one compound (starting material) of formula I at an initial concentration of 0.001 to 1 M,
   c) pH of the aqueous solution of 7 or more, in particular 8 or more, base concentration, concentration, in the range of up to 0.2 M , in particular 0.01 to 0.1 M, more particularly about 0.1M, wherein the base is selected from alkali metal and alkaline earth metal hydroxides or carbonates, in particular K₂CO₃, LiOH, NaOH, KOH, CsOH and Ba(OH)₂,
   d) temperature in the range of 0 to 80° C., in particular 20 to 60° C.
   e) terminal voltage in the range of 1 to 30 V,
   f) current density in the range of 0.5 to 500 mA/cm², in particular in the range of 1 to 50 mA/cm²; and
   g) applied charge Q in the range of 1 to 10 Farad, in particular 4 to 8 F.
   In particular, a combination comprising at least features a), b), c), f) and g) is applied.
21. The process of embodiment 20, wherein electrochemical oxidation of a compound of formula I is performed under at least one of the following conditions, which favor the formation of products of formula II, in particular of the formula XII, XXII, XXXII, more particularly XLII or LII, wherein predominantly Z is ═O:
   a) anode material selected from carbon fiber, graphite, Pt, GC or BDD, more particularly Pt and BDD,
   b) aqueous solution of at least one compound of formula I at an initial concentration as stated above
   c) pH of the aqueous solution of 7 or more, in particular 8 or more, base concentration, in the range of up to 0.2 M, in particular 0.01 to 0.15 M, more particularly 0.05 to 0.1 M; wherein base is selected from alkali and alkaline earth hydroxides or carbonates, in particular K₂CO₃, LiOH, NaOH, KOH and CsOH, more particularly NaOH,
   d) temperature in the range as stated above
   e) terminal voltage in the range of 1 to 30 V
   f) current density in the range as stated above; and
   g) applied charge Q in the range of 5 to 8 F.
   In particular, a combination comprising at least features a), b), c), f) and g) is applied.
22. The process of embodiment 20, wherein the electrochemical oxidation of a compound of formula I is performed under at least one of the following conditions which favor the formation of products of formula II, in particular of the formula XII, XXII, XXXII, more particularly XLII or LII, wherein predominantly Z is —OH:
   a) anode material selected from Ni, Cu or Au
   b) aqueous solution of at least one compound of formula I at an initial concentration as stated above
   c) pH of the aqueous solution of 7 or more, in particular 8 or more, base concentration, in particular NaOH concentration, in the range of up to 0.2 M , in particular 0.01 to 0.15 M, more particularly 0.05 to 0.1 M, wherein the base is selected from alkali metal and alkaline earth metal hydroxides or carbonates, in particular K₂CO₃, LiOH, NaOH, KOH, CsOH and Ba(OH)₂,
   d) temperature in the range as stated above
   e) terminal voltage in the range of 1 to 30 V
   f) current density in the range of as stated above; and
   g) applied charge Q in the range of 1 to 5 F.

In particular a combination comprising at least features a), b), c), f) and g) is applied.
23. The process of one of the embodiments 20 to 22, wherein the starting material is a compound of formula I, more particularly a compound of formula XI, XXI or XXXI, and more particularly a compound of formula XLI or LI.
   The electrochemical oxidation systems of embodiments 20 to 22 are suited to effect the regio-specific oxidation reaction of compounds of the general formula I, in particular of the general formulae XI, XXI, XXXI, and more particularly of the formulae XLI or LI.

### b) Electrochemical Recycling or Preparation of Perhalogenate Oxidant

24. The process of embodiment 3 or 5, further comprising the recovery and electrochemical recycling of the spent alkali perhalogenate oxidant, in particular of the electrochemical oxidation of an alkali halogenate back to an alkali perhalogenate oxidant, more particularly the electrochemical oxidation of an alkali iodate, especially sodium or potassium iodate, back to an alkali periodate oxidant, especially sodium or potassium periodate oxidant.
   The electrochemical recycling of sodium iodate to sodium periodate is preferred. In particular, the alkali halogenate, more particularly alkali iodate, especially sodium or potassium iodate, even more particularly sodium iodate, is isolated from the reaction mixture as described in more detail below. For example isolation by precipitation, in particular by applying a water soluble organic solvent, as for example alcohol precipitation is performed. More particularly, methanol or iso-propanol is added to form a precipitate. This precipitate may then be isolated, for example by filtration, optionally by decantation. The thus obtained halogenate, in particular iodate, even more particularly sodium iodate, is then subjected to the electrochemical recycling process.
   In analogy, the present invention allows the recycling of any alkali perhalogenate oxidant spent in any other chemical and/or biochemical oxidation reaction, and in particular the electrochemical oxidation of an alkali halogenate back to an alkali perhalogenate oxidant, and more particularly the electrochemical oxidation of an alkali iodate, especially sodium or potassium iodate, even more particularly sodium iodate, back to an alkali periodate oxidant, especially sodium or potassium periodate oxidant, even more particularly back to sodium periodate, which may then be utilized again in said chemical or biochemical oxidation process.
25. The process of embodiment 24, wherein the electrochemical recycling comprises an anodic oxidation of said alkali halogenate more particularly alkali iodate, especially sodium or potassium iodate, even more particularly sodium iodate, back to said alkali perhalogenate oxidant, especially back to an alkali periodate oxidant, like sodium or potassium periodate oxidant, even more particularly back to sodium periodate oxidant.
26. The process of embodiment 24 or 25, wherein a boron-doped diamond anode is applied.
27. The method of anyone of the embodiments 24 to 26, wherein the oxidation is performed under at least one of the following conditions:
   a) (1) aqueous solution of at least one alkali halogenate, more particularly alkali iodate, especially sodium or potassium iodate, at an initial concentration cₒ of 0.001 to 5 M, more preferably from 0.001 to 2,5 M or 0.001 to 2 M or 0.001 to 1 M, in particular from 0.01 to 1 M or 0.01 to 0.5 M, and specifically from 0.1 to 0.3 M; (2) initial molarity of the base in the alkaline solution in the range of 0.3 to 5 M or 0.5 to 5 M, preferably 0.6 to 4 M, 0.8 to 4 M or 0.6 to 3 M, in particular 0.9 to 2 M and specifically 1 M, (3) optionally ratio of base to halogenate being in the range of 10:1 to 1:1, more preferably from 8:1 to 2:1, in particular 6:1 to 3:1, more particularly the ratio of base to halogenate being at least 2.5:1 or higher, specifically 5:1 to 4:1, wherein the base is selected from alkali metal and alkaline earth metal hydroxides or carbonates, in particular K₂CO₃, LiOH, NaOH, KOH, CsOH and Ba(OH)₂, more preferably NaOH, KOH, and most preferred NaOH;
   b) pH of the aqueous solution of 7 or more, like at a pH of at least 8, preferably of at least 10, in particular of at least 12, more particular at least 13, and specifically of at least 14,
   c) temperature in the range of 0 to 80° C., more preferably from 10 to 60° C., in particular from 20 to 30° C. and specifically from 20 to 25° C.,
   d) voltage in the range of 1 to 30V particularly from 1 to 20 V and more particular from 1 to 10 V,
   e) current density j in the range of 10 to 500 mA/cm², like from 50 to 150 mA/cm², in particular from 80 to 120 mA/cm² and specifically of about 100 mA/cm²; and
   f) applied charge Q in the range of 1 to 10 Farad, more preferably from 2 to 6 F, in particular from 2.5 to 4 F, and specifically 2.75 to 3.5 Farad,
   In particular, the optimum current density j can be determined with respect to the type of electrolysis applied by a skilled person in the art. Batch or divided batch electrolyses, may use current densities in the range of 10 to 500 mA/cm². If the oxidation is to be performed in an electrolytic flow cell, the flow rate determines the maximum current density to be applicable. For example, in a flow cell with 48 cm² anode surface area, an anode-membrane gap of 1 mm, and a flow rate of 7.5 L/h, the optimal current density j may be determined to be in a range of about 400-500 mA/cm², and specifically about 416 mA/cm².
   In general, at higher flow rates or higher halogenate (like iodate) concentrations, the current density j may be higher, while at lower flow rates or lower halogenate (like iodate) concentrations, the current density must be lower to maintain current efficiency (CE).
   In a particular embodiment, the initial molarity cₒ of the base in the aqueous alkaline solution of the alkali halogenate is in the range of 0.3 to 5 M or 0.5 to 5 M, preferably 0.6 to 4 M, 0.8 to 4 M or 0.6 to 3 M, in particular 0.9 to 2 M and specifically 1 M. In particular the base is NaOH or KOH and the alkali halogenate is sodium or potassium iodate. More particularly the base is NaOH and the alkali halogenate is sodium iodate.
   In another particular embodiment, the pH of the aqueous solution is at least 12, at least 13, and specifically at least 14.
   In another particular embodiment, the initial concentration cₒ of the at least one alkali halogenate, more particularly alkali iodate, especially sodium or potassium iodate, in said aqueous solution is low and is in the range of 0.001 to 1 M, in particular from 0.01 to 0.5 M or 0.01 to 0.4 M , and specifically from 0.05 to 0.25 M.
   In another particular embodiment, the ratio of cₒ (NaOH): cₒ (NaIO₃) is set in the range of 10:1 to 1:1, preferably 8:1 to 2:1, in particular 6:1 to 3:1, specifically 5:1 to 4:1.
   In another particular embodiment, a feature combination comprising at least the above features a), b), e) and f) is applied. Here feature a) may comprise either features a(1) and a(2), or features a(2) and a(3), or more preferably features a(1), a(2) and a(3).
   In another particular embodiment, a feature combination comprising at least the above features a) and b) is applied. Here feature a) may comprise either features a(1) and a(2), or features a(2) and a(3), or more preferably features a(1), a(2) and a(3).
   According to a very particular embodiment thereof the alkali metal is sodium, and the recycled product is sodium periodate, obtained as sodium para-periodate.
   According to said very particular embodiment, the following particular parameters are applied alone or in combination:
      current density j in the range of 50 to 100 mA/cm² in batch electrolysis; or current density j in the range of 400 to 500 mA/cm² in flow electrolysis (as for example observed at a flow rate of 7.5 L/h and 48 cm² anode surface area)
      applied charge Q in the range of 3 to 4 F
      initial concentration cₒ (NaIO₃) of about 0.21 M
      initial concentration cₒ (NaOH) of about 1.0 M
      ratio of cₒ (NaIO₃):cₒ (NaOH) of about 1:5
   In a further particular embodiment of the iodate recycling process of the invention, the para-periodate as preferentially obtained by electrolysis is converted to meta-periodate.

For this purpose, after electrolysis, para-periodate is isolated from the anolyte as described in more detail below. The precipitate is obtained from the liquid phase in the anode chamber by filtration or decantation. The precipitation may be completed by usual means, for example by the addition of sodium hydroxide or by concentration of the solvent. In order to obtain meta-periodate said para-periodate is neutralized by addition of acid, in particular sulfuric or nitric acid and is then recrystallized in a manner known per se.

### c) Electrochemical Preparation of Perhalogenates

28. A process for the preparation of an alkali perhalogenate, in particular periodate, which process comprises the electrochemical anodic oxidation of an alkali halogenate, in particular iodate, to an alkali perhalogenate, in particular periodate, wherein in particular a boron-doped diamond anode is applied. The alkali cation is, in particular, selected from sodium or potassium, especially sodium.
29. The process of embodiment 28, wherein the anodic oxidation is performed under at least one of the following conditions:
   a) (1) aqueous solution of at least one alkali halogenate, in particular iodate, at an initial concentration of 0.001 to 5 M or 0.001 to 1 M, more preferably from 0.001 to 2 M, in particular from 0.01 to 1 M or 0.01 to 0.5 M or 0.01 to 0.4 M or 0.05 to 0.25 M, and specifically from 0.1 to 0.3 M or 0.1 to 0. 25 M. (2) initial molarity of the base in the alkaline solution in the range of 0.3 to 5 M, preferably 0.6 to 3 M, in particular 0.9 to 2 M and specifically 1 M; (3)optionally the ratio of base to halogenate being 10:1 or higher, or particularly in the range of 10:1 to 1:1, more particularly from 8:1 to 2:1, even more particular 6:1 to 3:1, and specifically 5:1 to 4:1, wherein the base is selected from alkali metal and alkaline earth metal hydroxides or carbonates, in particular K₂CO₃, LiOH, NaOH, KOH, CsOH and Ba(OH)₂, more preferably NaOH, KOH, and most preferred NaOH;
   b) pH of the aqueous solution of 7 or more, like at a pH of at least 8, preferably of at least 10, in particular of at least 12, at least 13 and specifically of at least 14,
   c) temperature in the range of 0 to 80° C., more preferably from 10 to 60° C., in particular from 20 to 30° C. and specifically from 20 to 25° C.,
   d) voltage in the range of 1 to 30V, particularly from 1 to 20 V and more particular from 1 to 10 V,
   e) current density in the range of 10 to 500 mA/cm², more preferably from 50 to 150 mA/cm², in particular from 80 to 120 mA/cm² and specifically of about 100 mA/cm²; or in the range of 10 to 1000 mA/cm^{2′} more preferably from 50 to 750 mA/cm², in particular from 100 to 500 mA/cm² and specifically of about 400 mA/cm; and
   f) applied charge Q in the range of 1 to 10 F, more preferably from 2 to 6 F, in particular from 2.5 to 4 F, and specifically 2.75 to 3.5 F.
   In particular, the optimum current density j can be determined with respect to the type of electrolysis applied by a skilled person in the art. Batch or divided batch electrolyses, use current densities in the range of 10 to 1000 mA/cm². If the oxidation is to be performed in an electrolytic flow cell, the flow rate determines the maximum current density to be applicable. For example, in a flow cell with 48 cm² anode surface area, an anode-membrane gap of 1 mm, and a flow rate of 7.5 L/h, the optimal current density may be determined to be in a range of about 400-500 mA/cm², and specifically about 416 mA/cm².
   In general, at higher flow rates or higher halogenate (like iodate) concentrations, the current density j may be higher, while at lower flow rates or lower halogenate (like iodate) concentrations, the current density must be lower to maintain current efficiency (CE).
   In a particular embodiment, the initial molarity cₒ of the base in the aqueous alkaline solution of the alkali halogenate is in the range of 0.3 to 5 M or 0.5 to 5 M, preferably 0.6 to 4 M, 0.8 to 4 M or 0.6 to 3 M, in particular 0.9 to 2 M and specifically 1 M. In particular the base is NaOH or KOH and the alkali halogenate is sodium or potassium iodate. More particularly the base is NaOH and the alkali halogenate is sodium iodate.
   In another particular embodiment, the pH of the aqueous solution is at least 12, at least 13 and specifically at least 14.
   In another particular embodiment, the ratio of cₒ (NaOH): cₒ (NaIO₃) is set in the range of 10:1 to 1:1, preferably 8:1 to 2:1, in particular 6:1 to 3:1, specifically 5:1 to 4:1.
   In another particular embodiment, the initial concentration cₒ of the at least one alkali halogenate, more particularly alkali iodate, especially sodium or potassium iodate, in said aqueous solution is low and is in the range of 0.001 to 1 M, in particular from 0.01 to 0.5 M or 0.01 to 0.4 M, and specifically from 0.05 to 0.25 M.
   In another particular embodiment, a feature combination comprising at least features a), b), e) and f) is applied. Here feature a) may comprise either features a(1) and a(2), or features a(2) and a(3), or more preferably features a(1), a(2) and a(3).
   In another particular embodiment, a feature combination comprising at least the above features a) and b) is applied. Here feature a) may comprise either features a(1) and a(2), or features a(2) and a(3), or more preferably features a(1), a(2) and a(3).
   According to a particular embodiment thereof the alkali metal is sodium, and the obtained product is sodium periodate, obtained as sodium para-periodate.
   According to said particular embodiment the following particular parameters are applied alone or in combination:
      current density j in the range of 50 to 100 mA/cm² in batch electrolysis; or a current density j in the range of 400 to 500 mA/cm² in flow electrolysis (as for example observed at a flow rate of 7.5 L/h, an anode-membrane gap of 1 mm, and 48 cm² anode surface area);
      applied charge Q in the range of 3 to 4 F
      initial concentration cₒ (NaIO₃) of about 0.21 M
      initial concentration cₒ (NaOH) of about 1.0 M
      ratio of cₒ (NaIO₃):cₒ (NaOH) of about 1:5
   In a further particular embodiment of the iodate preparation process of the invention, the para-periodate as preferentially obtained by electrolysis is converted to meta-periodate.
   For this purpose, after electrolysis, para-periodate is isolated from the anolyte as described in more detail below. The precipitate is obtained from the liquid phase in the anode chamber by filtration or decantation. The precipitation may be completed by usual means, for example by the addition of sodium hydroxide or by concentration of the solvent. In order to obtain meta-periodate said para-periodate is neutralized by the addition of acid, in particular sulfuric or nitric acid and then recrystallized in a manner known per se.

### Further Aspects and Embodiments of the Invention

In the following sections the above-mentioned particular embodiments are further described in more detail.

### 1. Chemical Oxidation of Heterocyclic Alpha-Amino Amide Compounds

### 1.1 Ruthenium-Catalyzed Regio-Specific and Stereo-Conserving Chemical Oxidation

According to one particular aspect of the present invention, a particular class of Ruthenium based oxidation catalyst systems are suitable for the regio-specific and stereo-conserving chemical oxidation of the pyrrolidine substrates of above formula I

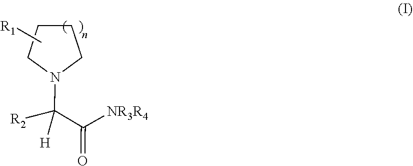

like in particular of (S)-2-(pyrrolidin-1-yl)butanamide (LIa).

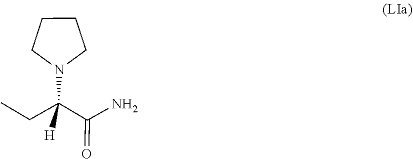

The catalyst may be a homogenous or a heterogeneous catalyst, as described in more detail below.

The chemical oxidation step is performed with a particular oxidation catalyst capable of oxidizing the heterocyclic alpha-amino group in a compound of formula Ia or Ib under substantial retention of the stereo configuration at the asymmetric carbon atom in alpha-position to the amine group to provide the final product in an essentially stereo-chemically pure form.

The oxidation catalyst according to this aspect of the invention is selected from combinations of an inorganic ruthenium (+III), (+IV), (+V), or (+VI), in particular (+III) or (+IV) salts and at least one oxidant capable of in situ oxidizing ruthenium (+III), (+IV), (+V), or (+VI), in particular (+III) or (+IV), in particular to ruthenium (+VIII), and optionally in the presence of a mono- or polyvalent metal ligand, as for example sodium oxalate.

Said inorganic ruthenium (+III) or (+IV) salt is selected from RuCl₃, RuO₂ and the respective hydrates, as for example monohydrates or higher hydrates, thereof.

Said inorganic ruthenium (+V) or (+VI) salt is selected from RuF₅ or RuF₆.

The oxidant may be selected from perhalogenates, hypohalogenites (in particular hypochlorite, NaClO), halogenates (in particular bromate, NaBrO₃) Oxone (KHSO₅.½KHSO₄. ½K₂SO₄), tert-butyl hydroperoxide (t-BuOOH), hydrogen peroxide (H₂O₂), molecular iodine (I₂), N-methylmorpholin-N-oxide, potassium persulfate (K₂S₂O₈), (Diacetoxyiodo)benzene, N-Bromosuccinimide, tert-butyl peroxybenzoate, iron(III) chloride or combinations thereof.

A preferred group of oxidants is selected from perhalogenates, preferably alkali perhalogenates, more preferably sodium or potassium perhalogenates, in particular sodium or potassium periodate, and specifically sodium meta-periodate or combinations thereof.

Another group of oxidants represents hypohalogenites and hydrates thereof, preferably alkali hypohalogenites, more preferably sodium or potassium hypohalogenites, in particular sodium or potassium hypochlorite pentahydrate, or combinations thereof.

Another group of oxidants represents combinations of the above described groups of hypohalogenites and perhalogenates.

### (i) Homogenous Oxidation Process

The oxidation reaction may be performed by dissolving the substrate of formula I in a suitable aqueous or organic solvent, either a non-polar aprotic, essentially water immiscible solvent, as for example carboxylic esters, like ethyl acetate, ethers or hydrocarbons (aliphatic or aromatic) or halogenated hydrocarbons (aliphatic or aromatic) or an organic solvent miscible with water, e.g. acetonitrile, acetone, N-methyl-2-pyrrolidone, or N,N-dimethylformamid. The solvent of the solution of the substrate of formula I preferably is selected from water, more preferably from a mixture of water and at least one of said organic solvents miscible with water, and even more preferably of at least one of said organic solvents or mixtures of at least two of said organic solvents. In another preferred embodiment, the substrate may be added neat.

Afterwards an aqueous solution or aqueous/organic solution mixture of the ruthenium salt and at least one oxidant for in situ oxidation of the ruthenium cation are added, optionally stepwise. Alternatively the aqueous or organic solution or aqueous/organic solution mixture of the substrate may be added, optionally stepwise, to the preformed aqueous solution or aqueous/organic solution mixture of the ruthenium salt and the at least one oxidant. The final solvent mixture is preferably composed of pure water, more preferably of a water/organic solvent mixture, in particular a mixture of water/acetone, water/ethyl acetate, water/acetonitrile, water/N-methyl-2-pyrrolidone, or water/N,N-dimethylformamid, and specifically water/acetonitrite. The final ratio of the water/organic solvent mixture is preferably from neat water to neat organic solvent, more preferably from 4:1 to 1:4 v/v, in particular 4:2 to 2:4 v/v, and specifically 1:1 v/v. Best results are obtained in two-phase solvent systems.

For performing the reaction, the initial substrate concentration may be chosen in a range depending on the solubility of the substrate in the respective solvent, as for example in a range of 0.001 to 1 mole/l. If the substrate is added neat, the initial substrate concentration is chosen in a range depending on the solubility of the substrate in the respective catalyst mixture, preferably in a range of 0.001 to 1 mole/l, more preferably from 0.01 to 0.5 mole/l, in particular from 0.1 to 0.2 mole/l, and specifically 0.107 mole/l. The substrate may also be added in amounts larger than the solubility product.

For performing the reaction, it is preferred to apply the oxidant in a molar excess over the substrate, preferably in a 1 to 10-fold, more preferably in a 1.1 to 5-fold, in particular in a 2 to 3-fold, and specifically in a 2.6-fold excess.

For performing the reaction, it is preferred to apply the ruthenium salt in catalytic amounts relative to the substrate, as for example in a range of 0.001 to100 mol %, preferably 0.005 to 10 mol %, in particular 0.05 to 1 mol %, and specifically 0.5 mol %.

The reaction is performed under stirring of the reaction mixture, or optionally the reaction may be performed without stirring. The generation of the active ruthenium catalyst may be aided by sonification.

The reaction is performed in an open or preferably closed reaction vessel.

The oxidation is carried out at pH value preferably between 2 and 12, more preferable between 4 and 10, in particular between 6 and 8, and specifically at pH 7.

The reaction temperature is chosen from a temperature in the range depending on the melting point of the respective solvent mixture, preferably from −20 to 80° C., more preferably −10 to 60° C., in particular −5 to 30° C., and specifically at 0° C.

After termination of the reaction, after about 5 to 400 minutes, preferably after 10 to 240 minutes, in particular after 20 to 60 minutes, and specifically after 30 minutes the reaction product may be isolated from the organic or the aqueous phase.

### (ii) Heterogeneous Oxidation Process

In another preferred embodiment, the stereospecific chemical oxidation of substrates of formula I, in particular of (S)-2-(pyrrolidin-1-yl)butanamide (LI) is performed in a continuous, heterogeneous method. While in the batch (or discontinuous; time-related) method the electrolyte containing the substrate is subjected to oxidation and after a certain time this is stopped and the product is isolated from the reaction vessel, in a continuous process design the substrate solution is passed continuously through a catalyst-containing material, preferably containing the catalyst in immobilized form.

For the immobilization, the said ruthenium salt is immobilized on an inert solid carrier material. The ruthenium salt, preferably, Ru(III)Cl or RuO₂, in particular the respective hydrates, and specifically ruthenium dioxide hydrate is mixed with the carrier material, as for example aluminum oxide, char coal, polyacrylonitrile (PAN), or alkylated silica, or combinations thereof. The mass of the ruthenium salt per 25 g carrier material ranges from preferably 1 mg to 5 g, more preferably from 50 mg to 2 g, in particular from 100 mg to 1 g, and specifically 200 mg. The said carrier material was loaded on a column. The size of the column may be chosen in a range depending on the substrate concentration and/or the scale of the oxidation process, as for example a diameter of 1.5 cm and a length of 15 cm. Various designs and geometries of columns are known in the art and can be applied to the present method.

The substrate of formula I and at least one oxidant are dissolved in pure water, in an organic solvent, or in solvent mixtures thereof. The same solvents and mixtures as described above for the homogeneous process may be applied.

The concentration of the substrate ranges preferably from 0.001 to 10 mole/l, more preferably from 0.01 to 5 mole/l, in particular 0.1 to 1 mole/l, and specifically 0.05 mole/l.

The solvent mixture ratio ranges from preferably neat water to 2:4 v/v water:organic solvent, more preferably from 4:1 to 1:4 v/v, in particular 4:2 to 2:4 v/v, and specifically 1:1 v/v.

The oxidant(s) is/are used in a molar excess over the substrate, preferably in a 1 to 10-fold, more preferably in a 1.1 to 5-fold, in particular in a 2 to3-fold, and specifically in a 2.6-fold excess.

For performing the reaction, the solution of the substrate is piped through the column by using a suitable pump or by another suitable pressure-generating arrangement. The flow rate is chosen in the range depending on the substrate concentration and/or the scale of the oxidation process, as for example 2 l/hand can easily adapted by one skilled in the art. The solution of the substrate may pass the column (material) once or multiple times.

The reaction temperature is chosen from a temperature in the range depending on the melting point of the respective solvent mixture, preferably from −20 to 80° C., more preferably −10 to 60° C., in particular −5 to 30° C., and specifically at 0° C. .

The oxidation is carried out at pH value preferably between 2 and 12, more preferable between 4 and 10, in particular between 6 and 8, and specifically at pH 7.

iii) Control of Product Composition

The composition of the reaction product resulting from the homogeneous or heterogeneous chemical oxidation of compounds of above general formula II may be controlled in various ways in order to obtain predominantly of a keto compound of the general formula IV

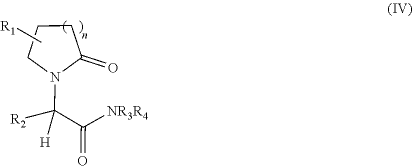

or particularly of a keto compound of the general formulae XIV, XXIV, XXXIV, XLIV or more particularly LIV (formulae see Section “General definitions” above)
or an alcohol of the general formula III,

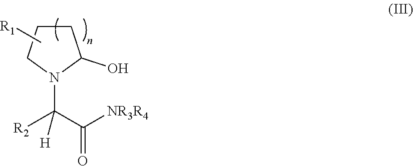

or particularly of an alcohol compound of the general formulae XIII, XXIII, XXXIII, XLIII or more particularly LIII (formulae see Section “General definitions” above)
or even a mixture of both types of compounds which may then be further purified.

The control of the reaction product composition, like a particular the conversion rate or the oxidation state of the constituents of the reaction product, is further illustrated by the numerous working examples in the experimental section below.

Activity of Ru-catalyst/reaction time: The catalytic activity of a ruthenium salt as applied as the catalyst may have influence on the composition of the reaction mixture. For example, the proportion of the keto product IV increases with increasing catalytic activity or with the duration of the oxidation reaction. Shorter reaction times or less catalytic activity favor the production of the corresponding alcohol product III.

Oxidant: The oxidation power of the applied oxidant as well as of the relative amount of oxidant also influences the product composition. For a preferential formation of the keto product IV the use of the perhalogenate, in particular periodate oxidant, in particular in a molar excess (as described above) is preferable.

Partial or complete replacement of the perhalogenate by hypohalogenites, like hypochlorite, favors the exclusive or the preferential formation of the corresponding alcohol product III.

Partial replacement of the perhalogenate by other oxidants like Oxone, T-Hydro, HIO₄, hypervalent iodides (DIB), KBrO₃ or combinations thereof, favors the formation of the corresponding alcohol product III in increasing proportions.

Complete replacement of the perhalogenate by other oxidants like HIO₄, hypervalent iodides (DIB),KBrO₃ or combinations thereof, favors the formation of the keto product IV, however in lower yields.

Complete replacement of the perhalogenate by other oxidants like Oxone, T-Hydro, KBrO₃ or combinations thereof, favors the formation of mixtures of the keto product IV and the corresponding alcohol product III.

Metal binding agent: The addition of a metal binding agent or ligand, like sodium oxalate chelating agent, further supports the formation of the keto product IV at further improved ee % values.

### 1.2 Iron-Catalyzed Regio-Specific and Stereo-Conserving Chemical Oxidation

According to another particular aspect of the present invention, a particular class of iron based oxidation catalyst systems are suitable for the regio-specific chemical oxidation of the pyrrolidine substrates of above formula I

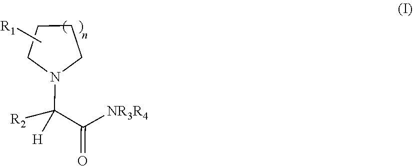

like in particular of (S)-2-(pyrrolidin-1-yl)butanamide LIa.

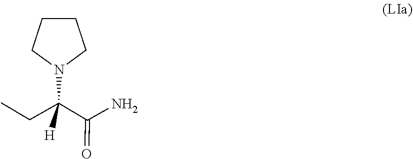

The catalyst may be a homogenous or a heterogeneous catalyst, in particular homogenous non-immobilized catalyst

The chemical oxidation of step is performed with particular oxidation catalyst capable of oxidizing the heterocyclic alpha-amino group in a compound of formula Ia or Ib at the asymmetric carbon atom in alpha-position to the amide group to provide the final product in an essentially stereo-chemically pure form.

The oxidation catalyst according to this aspect of the invention is selected from combinations of an inorganic iron (+II) and (+III) salts and at least one oxidant capable of in situ oxidizing said iron (+II) or (+III) salt to iron (+IV), (+V) or (+VI).

Said inorganic iron (+II) or (+III) salt is selected from FeCl₂, FeCl₃, FeSO₄ and the respective hydrates.

The oxidant may be selected from hydrogen peroxide, T-HYDRO, PhCO₃tBu and combinations thereof.

The oxidation reaction may be performed by dissolving the substrate of formula I in a suitable aqueous or organic solvent, either a non-polar aprotic, essentially water immiscible solvent, as for example carboxylic esters, like ethyl acetate, ethers or hydrocarbons (aliphatic or aromatic) or halogenated hydrocarbons (aliphatic or aromatic) or an organic solvent miscible with water, e.g. acetonitrile, acetone, N-methyl-2-pyrrolidone, or N,N-dimethylformamide. The solvent of the solution of the substrate of formula I preferably is selected from water, more preferably from a mixture of water and at least one of said organic solvents miscible with water, and even more preferably of at least one of said organic solvents or mixtures of at least two of said organic solvents. In another preferred embodiment, the substrate may be added neat.

Afterwards an aqueous solution or aqueous/organic solution mixture of the iron salt and at least one oxidant for in situ oxidation of the iron cation are added, optionally stepwise. Alternatively the aqueous or organic solution or aqueous/organic solution mixture of the substrate may be added, optionally stepwise, to the preformed aqueous solution or aqueous/organic solution mixture of the iron salt and the at least one oxidant. The final solvent mixture is preferably composed of pure water, more preferably of a water/organic solvent mixture, in particular a mixture of water/acetone, water/ethyl acetate, water/acetonitrile, water/N-methyl-2-pyrrolidone, or water/N,N-dimethylformamid, and specifically water/acetonitrile. The final ratio of the water/organic solvent mixture is preferably from neat water to neat organic solvent, more preferably from 4:1 to 1:4 v/v, in particular 4:2 to 2:4 v/v, and specifically 1:1 v/v.

For performing the reaction, the initial substrate concentration may be chosen in a range depending on the solubility of the substrate in the respective solvent, as for example in a range of 0.001 to 1 mole/l. If the substrate is added neat, the initial substrate concentration is chosen in a range depending on the solubility of the substrate in the respective catalyst mixture, preferably in a range of 0.001 to 1 mole/l, more preferably from 0.01 to 0.5 mole/l, in particular from 0.1 to 0.2 mole/l, and specifically 0.107 mole/l. The substrate may also be added in amounts larger than the solubility product.

For performing the reaction, it is preferred to apply the oxidant in a molar excess over the substrate, preferably in a 1 to 10-fold, more preferably in a 1.1 to 5-fold, in particular in a 2 to 3-fold, and specifically in a 2.6-fold excess.

For performing the reaction, it is preferred to apply the iron salt in catalytic amounts relative to the substrate, as for example in a range of 0.001 to100 mol %, preferably 0.005 to 10 mol %, in particular 0.05 to 1 mol %, and specifically 0.5 mol %.

The reaction is performed under stirring of the reaction mixture or optionally the reaction may be performed without stirring. The generation of the active iron catalyst may be aided by sonification.

The reaction is performed in an open or preferably closed reaction vessel.

The oxidation is carried out at pH value preferably between 2 and 12, more preferable between 4 and 10, in particular between 6 and 8, and specifically at pH 7.

The reaction temperature is chosen from a temperature in the range depending on the melting point of the respective solvent mixture, preferably from −20 to 80° C., more preferably −10 to 60° C., in particular −5 to 30° C., and specifically at 0° C. .

After termination of the reaction, after 5 to 400 minutes, preferably after 10 to 240 minutes, in particular after 20 to 60 minutes, and specifically after 30 minutes the reaction product may be isolated from the organic or the aqueous phase.

### 1.3 Further Catalysts for Chemical Oxidation

As further systems for the at least regio-specific oxidation of compounds of the formula I there may be mentioned:

### 1.3.1 I2 Oxidant in the Presence of Sodium Hydrogen Carbonate:

This oxidant system favors the formation of reaction mixtures predominantly or exclusively containing as reaction product of the alcohol compound of formula III.

The oxidation reaction is performed on the basis of Griffiths et al., Org. Lett. 2017, 19, 4, 870-873

The oxidation reaction may be performed by dissolving the substrate of formula I in a suitable aqueous or an organic solvent miscible with water, e.g. acetonitrile, acetone, N-methyl-2-pyrrolidone, N,N-dimethylformamide, DMSO and THF. The solvent of the solution of the substrate of formula I preferably is selected from water, more preferably from a mixture of water and at least one of said organic solvents miscible with water.

Afterwards an aqueous solution or aqueous/organic solution mixture of the oxidant are added, optionally stepwise. Alternatively, the aqueous or organic solution or aqueous/organic solution mixture of the substrate may be added, optionally stepwise, to the preform aqueous solution or aqueous/organic solution mixture of the oxidant.

For performing the reaction, the initial substrate concentration may be chosen in a range depending on the solubility of the substrate in the respective solvent, as for example in a range of 0.001 to 1 mole/l. If the substrate is added neat, the initial substrate concentration is chosen in a range depending on the solubility of the substrate in the respective catalyst mixture, preferably in a range of 0.001 to 1 mole/l, more preferably from 0.01 to 0.5 mole/l, in particular from 0.1 to 0.2 mole/l. The substrate may also be added in amounts larger than the solubility product.

For performing the reaction it is preferred to apply the oxidant and the sodium hydrogen carbonate independently in a molar excess over the substrate, preferably in a 1 to 10-fold, more preferably in a 1.1 to 5-fold, in particular in a 2 to 3-fold, excess.

The reaction is performed under stirring of the reaction mixture, or optionally the reaction may be performed without stirring.

The reaction is performed in an open or preferably closed reaction vessel.

The oxidation is carried out at pH value preferably between 2 and 12, more preferable between 4 and 10, in particular between 6 and 8, and specifically at pH 7.

The reaction temperature is chosen from a temperature in the range depending on the melting point of the respective solvent mixture, preferably from −20 to 80° C., more preferably −10 to 60° C., in particular −5 to 30° C., and specifically at 0° C. .

After termination of the reaction, after 5 to 400 minutes, preferably after 10 to 240 minutes, in particular after 20 to 60 minutes, and specifically after 30 minutes the reaction product may be isolated from the organic or the aqueous phase.

### 1.3.2 Au-Coated Aluminum Oxide in the Presence of Oxygen:

This oxidant system favors the regio-specific formation of reaction mixtures composed of the alcohol compound of formula III and the keto compound of formula IV.

As oxidation catalyst Au-coated aluminum oxide particles are applied.

The preparation of the Au/Al₂O₃-particles can be performed according to the modified procedure of Jin et al. Angewandte Chemie (Internatioal Edition) 2016, 55, 7212-7217, as described in the experimental part, below. The gold content of such particles may be in the range of 0.1 to 0.5, in particular about 0.25 mmole Au/mg particle.

The reaction is performed in the presence of molecular oxygen and under heating.

In particular, the reaction may be performed in open or closed reaction vessel under normal or increased oxygen pressure, as for example in a closed vessel and oxygen pressure in the range of 2 to 10 bar, in particular about 2 to 6 bar.

The reaction temperature may be set to a value in the range of 60-120, in particular 80-100° C.

The substrate of the general formula I, for example 2-(pyrrolidin-1-yl)butanamide of the formula LI, has to be dissolved in a suitable solvent or solvent mixture, in particular aqueous solvent or solvent mixture, more particularly water. The substrate concentration may be set to a value in the range of 0.01 to 1 mole, in particular 0.03 to 0.1 mole).

The Au/Al₂O₃-particles may be added in a proportion of 10 to 100, in particular 30-80 mg per equivalent of substrate.

The molar proportion of Agold relative to the molar amount of substrate may be set to a value in the range of about 1-5, in particular 2-3 mole % Au

The flask may be placed in an autoclave, pressurized with oxygen and heated, for example in an oil bath.

The duration of the reaction may be in the range of 1 or several hours and up to 5 days, as for example 1 to 4 days.

After running the reaction to completeness and cooling to room temperature the crude product can be isolated, for example by extraction with an organic solvent, like for example ethyl acetate. The particles can be recovered by filtration of the solution through a filter of suitable pore size.

The reaction product may be composed of a mixture of the respective alcohol of the formula III, as for example of the formula LIII, and the respective ketone of the formula IV, as for example of the formula LIV.

2. Electrochemical Recycling of Halogenate/Iodate to Perhalogenate/Periodate and the De Novo Synthesis of Perhalogenate/Periodate from Halogenate/Iodate

In another preferred embodiment, the produced halogenate, preferably iodate is recovered from the reaction mixtures of the oxidation process of a substrate of formula I, and the oxidation of halogenate/iodate to perhalogenate/periodate is performed electrochemically by anodic oxidation. A related process, i.e. the anodic oxidation of iodide to periodate at boron-doped diamond electrodes was described in a European patent application in the name of PharmaZell GmbH (EP 19214206.5, filing date Dec. 6, 2019).

However, it is well understood, that the recycling process of an alkali iodate according to the present invention is not limited to the particular process described herein with respect to the oxidation process of a substrate of above formula I. Alkali iodate, as formed form alkali periodate by any type of oxidation reaction, may be recycled to generate the alkali periodate oxidant. In such reaction media the initial concentration c₀ of the halogenate, more particularly of alkali iodate, especially of sodium or potassium iodate, may be in the range of 0.001 to 1 M, in particular from 0.01 to 0.5 M or 0.01 to 0.4 M, and specifically from 0.05 to 0.25 M. As non-limiting example cellulose processing industry, like paper industry may be mentioned as a technical field for applying the present process. In paper industry cellulose may be treated by oxidation. Cellulose is effectively oxidized to dialdehyde cellulose (DAC) by consumption of sodium periodate and formation of sodium iodate, which may then be recycled electrochemically according to the present invention.

The recovery of the iodate for the recycling meaning the isolation or the work-up of such from the reaction medium of periodate-based oxidations, preferably from the reaction mixture of the oxidation of substrates of formula I, depends on the desired product or the reaction conditions inter alia and are principally known to those skilled in the art.

For instance, to obtain the generated halogenate, preferably iodate, and in particular sodium iodate, the reaction medium is mixed with less polar water miscible solvents, preferably alcohols, carboxylic acids, carboxylic esters, ethers, amides, pyrrolidones, carbonates, tetramethylurea or nitriles, in particular ethanol, iso-propanol or methanol, acetic acid, ethyl acetate, tetrahydrofuran, N-methylpyrrolidone, N, N-dimethylformamide, N, N-dimethylacetamide, or acetonitrile to force precipitation. The precipitated halogenate can be isolated by usual means, such as filtration or decantation of the supernatant. If desired, the precipitate can then be subjected to further purification steps in order to remove undesired side products etc., if any, such as by washing with organic solvent (mixtures), or by recrystallization.

The electrolysis cell in which the anodic oxidation is carried out comprises one or more anodes in one or more anode compartments and one or more cathodes in one or more cathode compartments, where the anode compartments are preferably separated from the cathode compartments. If more than one anode is used, the two or more anodes can be arranged in the same anode compartment or in separate compartments. If the two or more anodes are present in the same compartment, they can be arranged next to each other or on top of each other. The same applies to the case that one or more cathodes are used. In case of two or more electrolysis cells, they can be arranged next to each other or on top of each other. The separation of the anode compartment(s) from the cathode compartment(s) can be accomplished by using different electrolysis cells for cathode(s) and anode(s) and connecting these cells by a salt bridge for charge equalization. The separators separate the anolyte that is the liquid medium in the anode compartment(s) from the catholyte that is the liquid medium in the cathode compartment(s), but allow charge equalization. Diaphragms are separators comprising porous structures of an oxidic material, such as silicates, e.g. in the form of porcelain or ceramics. Due to the sensitivity of diaphragm materials to harsher conditions, semipermeable membranes are however generally preferred, especially if the reaction is carried out at basic pH, as it is preferred. Membrane materials, which resist harsher conditions, especially basic pH, are based on fluorinated polymers. Examples for suitable materials for this type of membranes are sulfonated tetrafluoroethylene based fluoropolymer-copolymers, such as the Nafion® brand from DuPont de Nemours or the Gore-Select® brand from W.L. Gore & Associates, Inc. If the reaction is carried out in batch, the anode and cathode compartments are generally designed as batch cells. If the reaction is carried out semi-continuously or continuously, the anode and cathode compartments are generally designed as flow cells. Various designs and geometries of electrolysis cells are known to those skilled in the art and can be applied to the present method.

As anode (or electrode, more generally speaking) carbon-comprising materials may be used. Carbon-comprising anodes/electrodes are well known in the art and include for example graphite electrodes, vitreous carbon (glassy carbon) electrodes, reticulated vitreous carbon electrodes, carbon fiber electrodes, electrodes based on carbonized composites, electrodes based on carbon-silicon composites, graphene-based electrodes and boron diamond-based electrodes.

Electrodes are not necessarily composed entirely of the mentioned material, but may consist of a coated carrier material, for instance silicon, self-passivating metals, such as germanium, zirconium, niobium, titanium, tantalum, molybdenum and tungsten, metal carbides, graphite, glassy carbon, carbon fibers and combinations thereof.

Suitable self-passivating metals are for example germanium, zirconium, niobium, titanium, tantalum, molybdenum and tungsten.

Suitable combinations are for example metal carbide layers on the corresponding metal (such an interlayer may be formed in situ when a diamond layer is applied to the metal support), composites of two or more of the above-listed support materials and combinations of carbon and one or more of the other elements listed above. Examples for composites are siliconized carbon fiber carbon composites (CFC) and partially carbonized composites.

Preferably, the support material is selected from the group consisting of elemental silicon, germanium, zirconium, niobium, titanium, tantalum, molybdenum, tungsten, carbides of the eight aforementioned metals, graphite, glassy carbon, carbon fibers and combinations (in particular composites) thereof.

More preference is given to elemental silicon, germanium, zirconium, niobium, titanium, tantalum, molybdenum, tungsten and a combination of one of the seven afore-mentioned metals with the respective metal carbide.

Among the anode materials, preference is given to boron-doped diamond. The boron-doped diamond comprises boron in an amount of preferably 0.02 to 1% by weight (200 to 10,000 ppm), more preferably of 0.04 to 0.2% by weight, in particular of 0.06 to 0.09% by weight, relative to the total weight of the doped diamond.

As already indicated above, such electrodes are generally not composed of doped diamond alone. Rather, the doped diamond is attached to a substrate. Most frequently, the doped diamond is present as a layer on a conducting substrate, but diamond particle electrodes, in which doped diamond particles are embedded into a conducting or non-conducting substrate are suitable as well. Preference is however given to anodes in which the doped diamond is present as a layer on a conducting substrate.

Doped diamond electrodes and methods for preparing them are known in the art and described, for example, in the above-mentioned Janssen article in Electrochimica Acta 2003, 48, 3959, in NL1013348C2 and the references cited therein. Suitable preparation methods include, for example, chemical vapour deposition (CVD), such as hot filament CVD or microwave plasma CVD, for preparing electrodes with doped diamond films; and high temperature high pressure (HTHP) methods for preparing electrodes with doped diamond particles. Doped diamond electrodes are commercially available.

The cathode material is not very critical, and any commonly used material is suitable, such as stainless steel, chromium-nickel steel, platinum, nickel, bronze, tin, zirconium or carbon-comprising electrodes. In a specific embodiment, a stainless steel electrode is used as cathode.

Suitably, the electrochemical oxidation of the iodate is carried out in aqueous medium. Thus, the method of the invention comprises subjecting an aqueous solution comprising the iodate, in particular a metal iodate to anodic oxidation.

The electrolysis may be carried out under galvanostatic control (i.e. the applied current is controlled; voltage may be measured, but is not controlled) or potentiostatic control (i.e. the applied voltage is controlled; current may be measured, but is not controlled), the former being preferred.

In case of the preferred galvanostatic control, the observed voltage is generally in the range of from 1 to 30 V, more frequently from 1 to 20 V and in particular from 1 to 10 V.

In case of potentiostatic control, the applied voltage is generally in the same range, i.e. from 1 to 30 V, preferably from 1 to 20 V, in particular from 1 to 10 V.

The anodic oxidation is preferably carried out at a current density in the range of from 10 to 500 mA/cm², more preferably from 50 to 150 mA/cm², in particular from 80 to 120 mA/cm² and specifically of ca. 100 mA/cm².

To maximize the conversion of iodate to periodate, a charge of preferably at least 2 Farad, more preferably of at least 2.5 Farad, in particular of at least 2.75 Farad, and specifically of at least 3 Farad is applied. More particularly, a charge in the range of preferably 1 to 10 Farad, more preferably from 2 to 6 F, in particular from 2.5 to 5 F, and specifically 3 to 4 Farad is applied.

The electrolysis may be performed under acidic, neutral or basic conditions. Preferably the electrolysis is performed under basic conditions. Suitable bases to be used in the present method of the invention are all those which form hydroxide anions in the aqueous phase. Preferred are inorganic bases, such as metal hydroxides, metal oxides and metal carbonates, in particular alkali and earth alkali hydroxides. Preference is given to metal hydroxides where the metal of the base corresponds to the metal of the halogenate. The anodic oxidation is carried out at a pH of at least 8, preferably of at least 10, in particular of at least 12 and specifically of at least 14. Water is generally used as solvent.

The initial molarity of the halogenate, in particular iodate solution is preferably from 0.0001 to 10 M, more preferably from 0.001 to 5 M, in particular from 0.01 to 2 M, and specifically from 0.1 to 2 M. Low initial concentrations of the halogenate, in particular iodate solution, are of particular interest in such recycling processes, as iodate forming oxidation reactions may be performed in diluted reaction media.

The initial molarity of the base in the alkaline solution is 0.3 to 5 M, preferably 0.6 to 3 M, in particular 0.9 to 2 M and specifically 1 M.

The ratio of base to halogenate is10: 1 or higher, or preferably from 10:1 to 1:1, more preferably from 8:1 to 2:1, in particular 6:1 to 3:1, specifically 5:1 to 4:1.

If the oxidation reaction is carried out in batch, the above concentrations refer of course to the concentrations at the beginning of the reaction, since, as a matter of course, the concentration of the iodate decreases in the course of its conversion into the periodate. In case of a continuous design of the reaction, the above concentrations refer to the concentration in the aqueous medium continually introduced into the reaction. In case of a semi-continuous design of the reaction, the above concentrations refer to the concentration in the aqueous medium introduced in the course of the reaction.

The anodic oxidation is preferably carried out at a temperature of from 0 to 80° C., more preferably from 10 to 60° C., in particular from 20 to 30° C. and specifically from 20 to 25° C. The reaction pressure is not critical.

Under alkaline conditions, metal periodates are formed that are the metal salts of the various periodic acids. Periodate anions consist of an iodine in the oxidation state of +VII and include various structures, as for example ortho-periodate (IO₆⁵⁻), meta-periodate (IO₄⁻), paraperiodate (H₂IO₆³⁻), mesoperiodates (IO₅³⁻), or dimesoperiodates (I₂O₉⁴⁻) inter alia, depending on the pH of the medium. Meta-periodate, may be obtained specifically by acid recrystallization as described by C. L. Mehltretter, C. S. Wise, U.S. Pat. No. 2,989,371A, 1961, or H. H. Willard, R. R. Ralston, Trans. Electrochem. Soc. 1932, 62, 239.

Periodate in form of the para-periodate is isolated from the anolyte by filtration. If necessary the precipitation is forced by concentration of the solvent, by addition of less polar water-miscible solvents, by increasing the pH value, or by decreasing the temperature inter alia. Concentration, if required, can be carried out by usual means, such as evaporation of a part of the solvent, if desired under reduced pressure, partial freeze-drying, partial reverse osmosis etc. For the addition of water-miscible solvent, if required, preferably alcohols, carboxylic acids, carboxylic esters, ethers, amides, pyrrolidones, carbonates, tetramethylurea or nitriles, in particular ethanol, iso-propanol or methanol, acetic acid, ethyl acetate, tetrahydrofuran, N-methylpyrrolidone, N,N-dimethylformamide, N,N-dimethylacetamide, or acetonitrile are used. For increasing the pH value of the anodic media, if required, a suitable base, preferably metal hydroxides having a metal corresponding to the metal in the metal peroxohalogenate. The precipitated product can be isolated by usual means, such as filtration or decantation of the supernatant. Residual solvent in the product may be removed by usual means, such as evaporation, storing it in a desiccator etc., and, if desired, the product is crystallized and/or recrystallized.

Alternatively, the solvent can be removed from the reaction medium, for example by evaporation of the solvent, if desired under reduced pressure, freeze-drying, reverse osmosis, etc. The residue can be purified by usual means, e.g. recrystallization, chromatography, or extraction.

### 3. Electrochemical Oxidation of Heterocyclic Alpha-Amino Amide Compounds

In another preferred embodiment, the oxidized compounds of the general formula II, as for example compounds of the general formulae XII, XXII,)00(1, and more particularly of the formula XLII and LII, in particular those wherein Z is a keto group, may also be prepared electrochemically in a suitable electrolysis cell by anodic oxidation.

The electrolysis cell in which the anodic oxidation is carried out comprises one or more anodes in one or more anode compartments and one or more cathodes in one or more cathode compartments, where the anode compartments are preferably separated from the cathode compartments. If more than one anode is used, the two or more anodes can be arranged in the same anode compartment or in separate compartments. If the two or more anodes are present in the same compartment, they can be arranged next to each other or on top of each other. The same applies to the case that one or more cathodes are used. In case of two or more electrolysis cells, they can be arranged next to each other or on top of each other. The separation of the anode compartment(s) from the cathode compartment(s) can be accomplished by using different electrolysis cells for cathode(s) and anode(s) and connecting these cells by a salt bridge for charge equalization. The separators separate the anolyte that is the liquid medium in the anode compartment(s) from the catholyte that is the liquid medium in the cathode compartment(s), but allow charge equalization. Diaphragms are separators comprising porous structures of an oxidic material, such as silicates, e.g. in the form of porcelain or ceramics. Due to the sensitivity of diaphragm materials to harsher conditions, semipermeable membranes are however generally preferred, especially if the reaction is carried out at basic pH, as it is preferred. Membrane materials, which resist harsher conditions, especially basic pH, are based on fluorinated polymers. Examples for suitable materials for this type of membranes are sulfonated tetrafluoroethylene based fluoropolymer-copolymers, such as the Nafion® brand from DuPont de Nemours or the Gore-Select® brand from W.L. Gore & Associates, Inc. If the reaction is carried out in batch, the anode and cathode compartments are generally designed as batch cells. If the reaction is carried out semi-continuously or continuously, the anode and cathode compartments are generally designed as flow cells. Various designs and geometries of electrolysis cells are known to those skilled in the art and can be applied to the present method.

As anode (or electrode, more generally speaking) carbon-comprising materials may be used. Carbon-comprising anodes/electrodes are well known in the art and include for example graphite electrodes, glass-like carbon (vitreous carbon, glassy carbon, GLC) electrodes, reticulated vitreous carbon electrodes, carbon fiber electrodes, electrodes based on carbonized composites, electrodes based on carbon-silicon composites, graphene-based electrodes and boron diamond-based electrodes. Other anode materials are metal-based anode materials. Metals may be selected from nickel, platinum, copper, and gold. GLC, graphite, carbon fiber, BDD, and in particular platinum and BDD are preferred.

The proper choice of charge Q applied in the process under otherwise identical conditions may be used in order to predetermine the type of oxidation product predominantly formed during electrolysis.

For example, Q in the range of 1-5 F, particularly 1-4 F, favours the formation of alcohols of the general formula III, like in particular and alcohol of the formula LIII.

For example, Q in the range of 4-8 F, particularly 5-8 F, favours the formation of ketones of the general formula IV, in particular a ketone of the formula LIV.

Electrodes are not necessarily composed entirely of the mentioned material, but may consist of a coated carrier material, for instance silicon, self-passivating metals, such as germanium, zirconium, niobium, titanium, tantalum, molybdenum and tungsten, metal carbides, graphite, glass-like carbon, carbon fibers and combinations thereof.

Suitable self-passivating metals are for example germanium, zirconium, niobium, titanium, tantalum, molybdenum and tungsten.

Suitable combinations are for example metal carbide layers on the corresponding metal (such an interlayer may be formed in situ when a diamond layer is applied to the metal support), composites of two or more of the above-listed support materials and combinations of carbon and one or more of the other elements listed above. Examples for composites are siliconized carbon fiber carbon composites (CFC) and partially carbonized composites.

Preferably, the support material is selected from the group consisting of elemental silicon, germanium, zirconium, niobium, titanium, tantalum, molybdenum, tungsten, carbides of the eight aforementioned metals, graphite, glass-like carbon, carbon fibers and combinations (in particular composites) thereof.

More preference is given to elemental silicon, germanium, zirconium, niobium, titanium, tantalum, molybdenum, tungsten and a combination of one of the seven afore-mentioned metals with the respective metal carbide.

For the preparation of ketone products of the general formula IV, and in particular of the formula LIV, preference is given to platinum, as for example as platinum metal or metal sheet, or as a platinum-coated carrier material, as anode material.

The boron-doped diamond (BDD) comprises boron in an amount of preferably 0.02 to 1% by weight (200 to 10,000 ppm), more preferably of 0.04 to 0.2% by weight, in particular of 0.06 to 0.09% by weight, relative to the total weight of the doped diamond.

As already indicated above, such BDD electrodes are generally not composed of doped diamond alone. Rather, the doped diamond is attached to a substrate. Most frequently, the doped diamond is present as a layer on a conducting substrate, but diamond particle electrodes, in which doped diamond particles are embedded into a conducting or non-conducting substrate are suitable as well. Preference is however given to anodes in which the doped diamond is present as a layer on a conducting substrate.

Doped diamond electrodes and methods for preparing them are known in the art and described, for example, in the above-mentioned Janssen article in Electrochimica Acta 2003, 48, 3959, in NL1013348C2 and the references cited therein. Suitable preparation methods include, for example, chemical vapour deposition (CVD), such as hot filament CVD or microwave plasma CVD, for preparing electrodes with doped diamond films; and high temperature high pressure (HTHP) methods for preparing electrodes with doped diamond particles. Doped diamond electrodes are commercially available.

The cathode material is not very critical, and any commonly used material is suitable, such as stainless steel, chromium-nickel steel, platinum, nickel, bronze, tin, zirconium or carbon-comprising electrodes. In a specific embodiment, a stainless-steel electrode is used as cathode.

Suitably, the electrochemical oxidation of the iodate is carried out in aqueous medium. Thus, the method of the invention comprises subjecting an aqueous solution comprising the iodate, in particular a metal iodate to anodic oxidation.

The electrolysis may be carried out under galvanostatic control (i.e. the applied current is controlled; voltage may be measured, but is not controlled) or potentiostatic control (i.e. the applied voltage is controlled; current may be measured, but is not controlled), the former being preferred.

In case of the preferred galvanostatic control, the observed voltage is generally in the range of from 1 to 30 V, more frequently from 1 to 20 V and in particular from 1 to 10 V.

In case of potentiostatic control, the applied voltage is generally in the same range, i.e. from 1 to 30 V, preferably from 1 to 20 V, in particular from 1 to 10 V.

The anodic oxidation is preferably carried out at a current density in the range of from 2 to 500 mA/cm², more preferably from 2 to 25 mA/cm², in particular from 2 to 10 mA/cm² and specifically of ca. 2 to 5 mA/cm².

To maximize the formation of the alcohol oxidation product of the general formula III, in particular of the formula LIII a charge of preferably at least 2 Farad, more preferably of at least 2.5 Farad, in particular of at least 2.75 Farad, and specifically of at least 3 Farad is applied. More particularly, a charge in the range of preferably 1 to 10 Farad, more preferably from 2 to 6 F, in particular from 2.5 to 4 F, and specifically 2.75 to 3.5 Farad is applied

To maximize the formation of the keto oxidation product of the general formula IV, in particular of the formula LIV, a charge of preferably at least 3,5 Farad, more preferably of at least 4 Farad, in particular of at least 4,5 Farad, and specifically of at least 5 Farad is applied. More particularly, a charge in the range of preferably 1 to 10 Farad, more preferably from 3 to 7 F, in particular from 4 to 6 F, and specifically 5.5 to 6 Farad is applied.

The electrolysis may be performed under acidic, neutral or basic conditions. Preferably, the electrolysis is performed under basic conditions. Suitable bases to be used in the present method of the invention are all those which form hydroxide anions in the aqueous phase. Preferred are inorganic bases, such as metal hydroxides, metal oxides and metal carbonates, in particular alkali and earth alkali hydroxides. Preference is given to metal hydroxides where the metal of the base corresponds to the metal of the halogenate. The anodic oxidation is carried out at a pH of at least 8, preferably of at least 10, in particular of at least 12 and specifically of at least 14. Water is generally used as solvent.

The initial molarity of the substrate solution of a substrate of the general formula I, as for example of the general formula XI, XXI, XXXI, or more particularly of the formulae XLI and LI, is preferably from 0.0001 to 10 M, more preferably from 0.001 to 5 M, in particular from 0.01 to 2 M, and specifically from 0.1 to 1 M.

The initial molarity of the base in the alkaline solution is 0.1 to 5 M, preferably 0.1 to 3 M, in particular 0.1 to 1 M and specifically 0.1 M.

The ratio of base to substrate is preferably from 10:1 to 1:1, more preferably from 5:1 to 1:1, in particular 2:1 to 1:1, specifically 1:1.

If the oxidation reaction is carried out in batch, the above concentrations refer of course to the concentrations at the beginning of the reaction, since, as a matter of course, the concentration of the substrate and reagents decrease in the course of its conversion. In case of a continuous design of the reaction, the above concentrations refer to the concentration in the aqueous medium continually introduced into the reaction. In case of a semi-continuous design of the reaction, the above concentrations refer to the concentration in the aqueous medium introduced in the course of the reaction.

The anodic oxidation is preferably carried out at a temperature of from 0 to 80° C., more preferably from 10 to 60° C., in particular from 20 to 30° C. and specifically from 20 to 25° C. The reaction pressure is not critical.

The oxidation product(s) is (are) isolated from the anolyte in conventional manner, as for example extraction with an organic solvent or chromatography, or as described in the following section.

### 4. Product Isolation

The methodology of the present invention can further include a step of recovering an end or intermediate product, optionally in stereoisomerical or enantiomerically substantially pure form.

The term “recovering” includes extracting, harvesting, isolating or purifying the compound from culture or reaction media. Recovering the compound can be performed according to any conventional isolation or purification methodology known in the art including, but not limited to, treatment with a conventional resin (e.g., anion or cation exchange resin, non-ionic adsorption resin, etc.), treatment with a conventional adsorbent (e.g., activated charcoal, silicic acid, silica gel, cellulose, alumina, etc.), alteration of pH, solvent extraction (e.g., with a conventional solvent such as an alcohol, ethyl acetate, hexane and the like), distillation, dialysis, filtration, concentration, crystallization, recrystallization, pH adjustment, lyophilization and the like, as well as combinations thereof. Identity and purity of the isolated product may be determined by known techniques, like High Performance Liquid Chromatography (HPLC), gas chromatography (GC), Spectroscopy (like IR, UV, NMR), Staining methods, TLC, NIRS, enzymatic or microbial assays.(see for example: Patek et al. (1994) Appl. Environ. Microbiol. 60:133-140; Malakhova et al. (1996) Biotekhnologiya 11 27-32; und Schmidt et al. (1998) Bioprocess Engineer. 19:67-70. Ullmann's Encyclopedia of Industrial Chemistry (1996) Bd. A27, VCH: Weinheim, S. 89-90, S. 521-540, S. 540-547, S. 559-566, 575-581 und S. 581-587; Michal, G (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, Bd. 17.)

In all embodiments of the claimed process described herein, the isolation or the workup of a product depend on the desired product and the reaction conditions inter alia and are principally known to those skilled in the art.

For instance, to obtain the oxidation product of formula IV, specifically (S)-α-ethyl-2-oxopyrrolidine acetamide (LIVa) from a Ru-based chemical oxidation reaction as described above, the reaction is stopped and the RuO₂ is precipitated by addition of an alcohol or any suitable oxidizable substance. The reaction mixture is filtrated trough a suitable porous material, such as neutral aluminum oxide or char coal. The filter cake is washed with additional water and a suitable organic solvent. The generated iodate and residues of periodates in the filtrate are removed by precipitation. The precipitation is forced by less polar water miscible solvents or by reducing the temperature; if necessary after concentration of the reaction medium. Concentration, if required, can be carried out by usual means, such as evaporation of a part of the solvent, if desired under reduced pressure, partial freeze-drying, partial reverse osmosis etc. The precipitated product can be isolated by usual means, such as filtration or decantation of the supernatant. The solvent of the product-containing solution is then concentrated or removed by usual means, such as evaporation, etc., and, if desired, the product is crystallized and/or recrystallized.

Alternatively, the solvent can be removed from the reaction medium, for example by evaporation of the solvent, if desired under reduced pressure, freeze-drying, reverse osmosis, etc. The residue can be purified by usual means, e.g. recrystallization, chromatography, or extraction.

If appropriate the reaction product may further processed by further purifying a particular stereoisomer, in case the product is composed of a mixture of two or more stereoisomers, as for example (S)- and (R)-enantiomers by applying conventional preparative separation methods like chiral chromatography or by resolution.

The intermediates and final products produced in any of the method described herein can be converted to derivatives such as, but not limited to esters, glycosides, ethers, epoxides, aldehydes, ketones, or alcohols. The derivatives can be obtained by a chemical method such as, but not limited to oxidation, reduction, alkylation, acylation and/or rearrangement. Alternatively, the compound derivatives can be obtained using a biochemical method by contacting the compound with an enzyme such as, but not limited to an oxidoreductase, a monooxygenase, a dioxygenase, a transferase. The biochemical conversion can be performed in-vitro using isolated enzymes, enzymes from lysed cells or in-vivo using whole cells. The following examples are illustrative only and are not intended to limit the scope of the embodiments described herein.

The numerous possible variations that will become immediately evident to a person skilled in the art after heaving considered the disclosure provided herein also fall within the scope of the invention.

### Experimental Section—Part A

### 1. APPARATUSES AND DEVICES

Electrochemical reactions were carried out at boron-doped diamond (BDD) anodes. The BDD electrodes were obtained in DIACHEM© quality from CONDIAS GmbH, Itzehoe, Germany. The BDD had a 15 μm diamond layer on silicon support. Stainless steel of the type EN1.4401; AISI/AS™ was used as cathodes. Nafion™ from DuPont was used as membrane. A galvanostate HMP4040 from Rhode&Schwarz was employed.

NMR spectra were recorded on a Bruker Avance III HD 300 (300 MHz) equipped with 5 mm BBFO head with z gradient and ATM at 25° C. Chemical shifts (δ) are reported in parts per million (ppm) relative to traces of CHCl₃ in CDCl₃ as deuterated solvent.

Liquid chromatography photodiode array analysis (LC-PDA) was performed by using a DUGA-20A₃ device from Shimadzu, which was equipped with a C18 column from Knauer (Eurospher II, 100-5 C18, 150×4 mm). The column was conditioned to 25° C. and the flow rate was set to 1 mL/min. The aqueous eluent was buffered with formic acid (0.8 mL/2.5 L) and stabilized with Acetone (5 vol %).

Gas chromatography (GC) was performed using a GC 2010 device from Shimadzu, which was equipped with a Varian capillary column ZB-5MSi (Serial No. 334634), operating with H2 as carrier gas. Infrared spectra were recorded on an ATR IR device of the type ALPHA from Bruker.

Thin Layer Chromatography (TLC) was performed using commercially available aluminum plates coated with silica.

Cyclic voltammetry (CV) was conducted on an AUTO LAB PGstat 204 from Metrohm AG, Herisau, Switzerland. Design of Experiments plans were planned and analyzed with the software Minitab19 from Minitab Inc.

Electrolysis cells were manufactured in the work shop of the chemistry department of the Johannes Gutenberg University Mainz and are commercially available as parts of the IKA Screening System at IKA®-Werke GmbH&CO. KG, Staufen, Germany. The IKA company also sells the 2×6 cm²-flow electrolysis cell as ElectraSyn Flow device. The stainless-steel flow electrolysis cell was purchased from CONDIAS GmbH, Itzehoe, Germany.

### 2. CHEMICALS

| | |
|---|---|
| (S)-2-Aminobutanamide | abcr GmbH, 98% |
| hydrochloride [7682-20-4] | |
| 1,4-Dibromobutane [110-52-1] | Merck-Schuchardt |
| Acetonitrile [75-05-8] | Fisher Scientific, ≥99.9% |
| Dichloromethane [75-09-2] | Fisher Scientific, ≥99.8% |
| Levetiracetam [102767-28-2] | Acros Organics |
| Magnesium sulphate [7487-88-9] | Acros Organics, 97% |
| Methanol [67-56-1] | VWR Chemicals, 100.0% |
| Sodium bisulfate | Merck, >99%; Acros Organics, 99+% |
| monohydrate [10034-88-5] | |
| Sodium thiosulfate | Acros Organics, 99.5% |
| pentahydrate [10102-17-7] | |
| Sodium carbonate [497-19-8] | Acros Organics, 99.5% |
| Sodium hydroxide [1310-73-2] | Honeywell, Pellets, ≥98%; |
| | VWR Chemicals, Pellets, 99.2% |
| Sodium iodate [7681-55-2] | Alfa Aesar, 99% |
| Sodium periodate [7790-28-5] | Fisher Scientific, 99% |
| Sodium sulphate [7757-82-6] | Merck |
| Phosphorus(V)-oxid [1314-56-3] | Alfa Aesar, 98% |
| Ruthenium(IV)-oxid | Aldrich |
| hydrate [12036-10-1] | |
| Nitric acid (≥65%) [7697-37-3] | Sigma-Aldrich |
| Hydrochloric acid | Fisher Scientific |
| (~37%) [7647-01-0] | |
| Sulphuric acid (>95%) [7664-93-9] | Fisher Scientific |
| Caffeine [58-08-2] | BASF (donation) |

All chemicals as used herein, except for those who were synthesized internally (as described herein), were of analytical grade and are obtained from commercial suppliers.

A stock solution of RuCl₃ (Alfa Aesar 47182, 7.9 mg) in H₂O (10 mL) was prepared daily to be used freshly (1 mL used for each reaction contained 0.79 mg RuCl₃*xH₂O).

### 3. METHODS

### 3.1 Gas Chromatography:

Conditions: 150° C.-5 min-25° C./min-300° C.-5 min; T^{a} det: 300° C; T^{a} inj: 220° C.; split 50:1; flux: 1.5 mL/min; carrier: He

Column: HP-5; 5% Phenyl Methyl Siloxane; 30 m, 0.2 mm ID, 0.33 μm

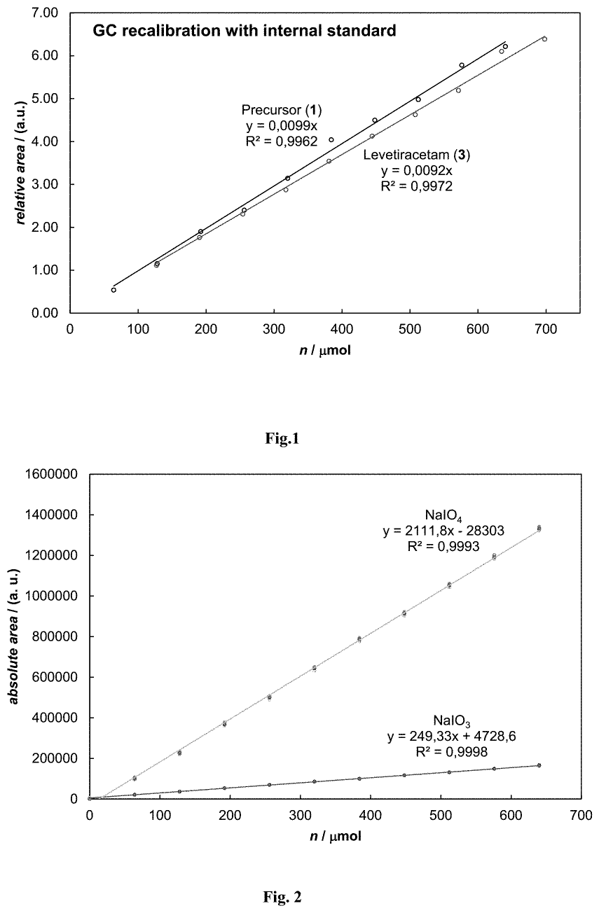

5 mg/mL in MeOH, method: Injection volume=1.5 μL, inlet temperature=200° C., initial column temperature=50° C. (holding time=1 min), ramping rate=15° C./min (gradient time=11.5 min), final temperature=220° C. (hold-up time=12 min). The system was calibrated for the precursor and for Levetiracetam using caffeine as internal standard (FIG. 1).

### 3.2 Liquid Chromatography Photodiode Array (LC-PDA)

A buffered and diluted sample solution (C=5.341 mM) was subjected to LC-PDA analysis using an injection volume of 3 μL. The separation was isocratically carried out. I⁻, IO₃⁻ and IO₄⁻ were detected by the PDA detector at 1.58 min,1.47 min and 1.92 min at a wavelength of λ=254 nm. Yields were determined by external calibration (FIG. 2).

### 3.3 Chiral HPLC

Conditions: Heptane: EtOH (90:10), 25° C., λ=215 nm, flux: 1.0 mL/min

Column: Chiralpak ADH 250×4.6 mm, 5 u

1 mg/mL in Heptan:EtOH

Chiral HPLC was performed with a Waters 2695 separation module with UV detector (Waters 996 photodiode array detector) with a CHIRALPAK IB-3 column (250×4.6 mm, particle size 3 μm, flow rate: 1.0 mL/min) and a guard column (10×4.0 mm) from Daicel Chiral Technologies. The system was operated with an isocratic program. The injection volume was V=10 μL, and the eluent was composed of 10% isopropanol and 90% hexane/ethanol. The detection followed by a photodiode array detector at λ=210.1 nm.

### 3.4 TLC

Thin Layer Chromatography (TLC) was performed using commercially available aluminum plates from Merck coated with silica (60 F₂₅₄) or 60-RP-18 F254 reversed-phase plates on aluminum from Merck KGaA. All samples were applied after dilution in a suitable solvent with ring caps 1-5 μL obtained from company Hirschmann and the chromatography was carried out in an eluent mixture. The TLC plate was viewed under UV light (λ=254 nm and 365 nm) and then developed in an iodine chamber or with coloring reagents and a heat gun:
Ninhydrin reagent: 0.3 g ninhydrin in 2.0 mL glacial acetic acid and 100 mL methanol
Dragendorff-Munier reagent: 20.0 g potassium iodide, 3.0 g bismuth (III) nitrate, 40.0 g (+)-tartaric acid and 240 mL water
KMnO₄ reagent: 3.0 g potassium permanganate and 20.0 g sodium carbonate in 300 mL water and 5.0 mL 5% sodium hydroxide solution
Seebach reagent: 10.0 g cerium (IV) sulfate, 25.0 g phosphoromolybdic acid, 940 mL water and 60 mL conc. sulfuric acid
Vanillin reagent: 1.0 g vanillin, 100 mL methanol, 12.0 mL glacial acetic acid and 4.0 mL conc. sulfuric acid
Dinitrophenylhydrazine reagent: 1.0 g 2,4-dinitrophenylhydrazine, 25 mL abs. ethanol, 8.0 mL water and 5.0 mL conc. sulfuric acid.
p-anisaldehyde reagent: 4.1 mL p-anisaldehyde, 5.6 mL conc. sulfuric acid, 1.7 mL glacial acetic acid and 150 mL ethanol
Bromocresol green reagent: 50 mg bromocresol green, 250 mL isopropanol and 0.15 mL 2 M sodium hydroxide solution.

### 4. EXAMPLES

### 4.1 Representative Procedures (RP) Applied in the Subsequent Section Ruthenium-Catalyzed Synthesis of Levetiracetam (RP 1)

0.5-1 mol % RuO₂.xH₂O (0.5-1.0 mg, 3.20-6.40 μmol) and 2.60 eq. of NaIO₄ (356 mg, 1.66 mmol) were suspended in a suitable solvent mixture (6 mL) until the solution showed a pale-yellow color. 1 (100 mg, 640 μmol) was added and the reaction was stirred at room temperature for 0.5 h. The reaction was controlled by GC versus caffeine (24.9 mg, 128 μmol, 0.20 eq.) as internal standard.

### Levetiracetam Synthesis on Immobilized Ruthenium Dioxide (RP 2)

For the catalyst immobilization, RuO₂.xH₂O (200 mg) was mixed with aluminum oxide, C18 reversed phase material, polyacrylonitrile, char coal, or mixtures thereof (25 g). The prepared material was loaded on a glass column (12×1.5 cm) and the column was connected to a Fink pump (Ritmo R033). For the oxidation, 1 (100 mg, 640 μmol) and 2.60 eq. of NaIO₄ (356 mg, 1.66 mmol) were dissolved in water/acetonitrile (2:1 v/v, 25 mL) and the solution was pumped through the column. The system was rinsed with another 10 mL of water.

### Electrochemical Synthesis of Etiracetam (RP 3)

In a divided batch-electrolysis cell equipped with a Nafion membrane, a stainless-steel cathode, and a platinum anode, 1 (100 mg, 0.64 μmol) was electrolyzed in caustic soda (0.1 M, 6 mL) using a current density of j=2 mA/cm² and an applied charge of Q=6 F. After electrolysis, the yield was determined by GC analysis versus caffeine as internal standard and the crude product was purified by flash column chromatography on silica gel.

### Electrochemical Recycling of Sodium Iodate (RP 4)

In a divided batch-electrolysis cell equipped with a Nafion membrane, a stainless-steel cathode, and a boron-doped diamond (BDD) anode, NaIO₃ (127 mg, 640 μmol) was electrolyzed in caustic soda (0.1 M) using a charge amount of Q=3 F, and a current density of j=10 mA/cm². After complete electrolysis the yield was determined by LC-PDA

4.2 Screening Examples for the Synthesis of Levetiracetam with Non-Immobilized Catalyst RuO₂.xH₂O/NaIO₄

### Screening Example 1: Solvent Screening

According to RP 1, RuO₂.xH₂O and NaIO₄ were suspended. 1 was added and the reaction was stirred at room temperature for 0.5 h.

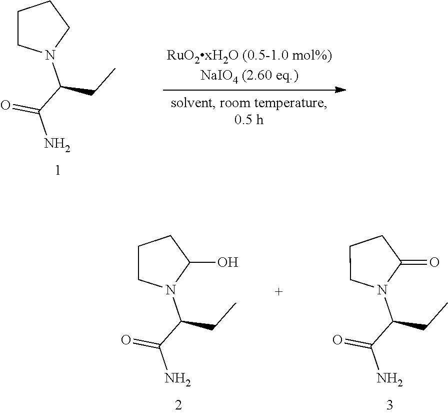

The results are summarized in Table 3.

TABLE 3

| Exp. | | Ratio | GC Yield [%] | | | |
|---|---|---|---|---|---|---|
| No. | Solvent | [v/v] | 1 | 2^{a} | 3 | Σ |
| 1 | H₂O | pure | 5 | 9 | 31 | 45 |
| 2 | H₂SO₄ | 0.1M | 16 | 0 | 9 | 25 |
| 3 | NaOH | 0.1M | 29 | 16 | 24 | 69 |
| 4 | H₂O/EtOAc | 4:2 | 3 | 5 | 32 | 40 |
| 5 | H₂O/MeCN | 4:2 | 1 | 4 | 57 | 62 |
| 6 | H₂O/acetone | 4:2 | 1 | 4 | 60 | 65 |
| 7 | H₂O/CHCl₃ | 4:2 | 2 | 8 | 17 | 27 |
| 8 | H₂O/CH₂Cl₂ | 4:2 | 3 | 5 | 21 | 29 |
| 9 | H₂O/NMP | 4:2 | 41 | 21 | 28 | 89 |
| 10 | H₂O/DMSO | 4:2 | 110 | 0 | 0 | 110 |
| 11 | H₂O/AcOH | 4:2 | 19 | 1 | 10 | 30 |
| 12 | H₂O/DMF | 4:2 | 16 | 18 | 37 | 72 |
| 13 | Acetone | pure | 132 | 1 | 0 | 133 |
| 14 | MeCN | pure | 102 | 0 | 0 | 102 |
| 15 | H₂O/Acetone/NMP | 4:2:0.5 | 5 | 14 | 64 | 83 |
| 16 | 0.1M NaOH/Acetone/NMP | 4:2:0.5 | 21 | 20 | 38 | 79 |
| 17 | 0.1M NaOH/MeCN/NMP | 4:2:0.5 | 16 | 15 | 42 | 73 |
| 18 | 0.1M H₂SO₄/MeCN/NMP | 4:2:0.5 | 3 | 1 | 27 | 31 |

^{a}relative area vs. standard

The product 3 and the intermediate 2 were isolated by flash column chromatography on silica gel (12×2 cm) using an eluent mixture of CH₂Cl₂/MeOH=10:1; or by crystallization in Et₂O or PE/CH₂Cl₂ at −20° C. Levetiracetam was isolated in 49% yield and in 99.6% ee.

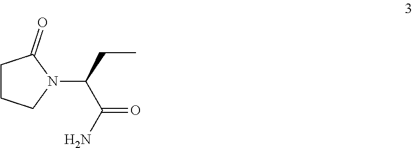

TLC (SiO₂, ninhydrin stain, strong heating), R_{f} (CH₂Cl₂/MeOH=10:1)=0.56, R_{f} (CH₂Cl₂/MeOH=20:1)=0.13; GC: R_{f}=8.98 min at ˜180° C.; LC-MS (HR): calculated for C₈H₁₄N₂O₂ 170.1055 Da, found: [M+H]⁺ 171.1128; ¹H NMR (400 MHz, chloroform-d) δ 6.58 (s, 1H), 6.11-5.88 (m, 1H), 4.46 (dd, J=9.1, 6.6 Hz, 1H), 3.41 (dddd, J=34.1, 9.8, 8.0, 6.1 Hz, 2H), 2.47-2.29 (m, 2H), 2.10-1.85 (m, 3H), 1.65 (ddq, J=14.6, 9.1, 7.4 Hz, 1H), 0.86 (t, J=7.4 Hz, 3H); ¹³C NMR (101 MHz, chloroform-d) δ 176.10 (C_{q}), 172.64 (C_{q}), 56.07 (CH), 43.89 (CH₂), 31.15 (CH₂), 21.21 (CH₂), 18.20 (CH₂), 10.59 (CH₃).

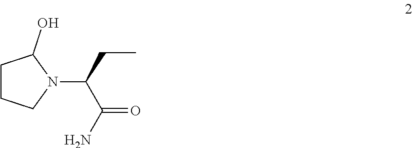

Characterized as a mixture of 2 and 3 (7:1): TLC (SiO₂, ninhydrin stain, medium heating), Rf(CH₂Cl₂/MeOH=10:1)=0.48; GC: R_{f}=8.20 min at −160° C.; LC-MS (HR ESI(+)): calculated for C₈H₁₆N₂O₂ 172.1212 Da, found: [M−OH]⁺ 155.1181; ¹H NMR (400 MHz, chloroform-d) δ 6.81 (s, 1H), 4.87 (ddd, J=5.7, 3.6, 1.4 Hz, 1H), 3.17 (dt, J=10.1, 5.7 Hz, 1H), 3.08 (ddd, J=8.0, 4.3, 1.5 Hz, 1H), 2.71 (ddd, J=10.0, 7.6, 6.3 Hz, 1H), 2.15-1.50 (m, 6H), 1.24 (s, 0H), 1.02 (t, J=7.4 Hz, 3H); ¹³C NMR (101 MHz, chloroform-d) δ 177.99 (Cq), 75.18 (CH), 68.93 (CH), 56.55 (CH₂), 32.87 (CH₂), 25.99 (CH₂), 23.84 (CH₂), 10.45 (CH₃).[SAR018]

### Screening Example 2: pH Screening

According to RP 1, RuO₂.xH₂O and NaIO₄ were suspended. 1 was added and the reaction was stirred at room temperature for 0.5 h.

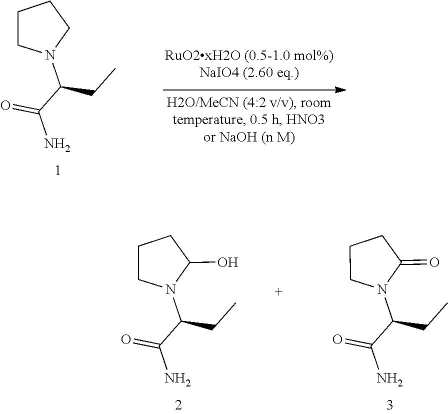

The results are summarized in Table 4.

TABLE 4

| | | |
|---|---|---|
| Exp. | M | GC Yield [%] |

| No. | acid or base | [M] | 1 | 2^{a} | 3 | Σ |
|---|---|---|---|---|---|---|
| 1 | HNO₃ | 0.01 | 0 | 3 | 33 | 36 |
| 2 | HNO₃ | 0.1 | 0 | 4 | 17 | 21 |
| 3 | HNO₃ | 0.5 | 0 | 0 | 0 | 0 |
| 4 | HNO₃ | 2.0 | 0 | 0 | 0 | 0 |
| 5 | HNO₃ | 3.0 | 0 | 0 | 0 | 0 |
| 6 | NaOH | 0.01 | 0 | 5 | 40 | 45 |
| 7 | NaOH | 0.1 | 0 | 15 | 24 | 39 |
| 8 | NaOH | 0.5 | 51 | 16 | 0 | 67 |
| 9 | NaOH | 2.0 | 63 | 6 | 0 | 69 |
| 10 | NaOH | 3.0 | 75 | 4 | 0 | 79 |

^{a}relative area vs. standard

### Screening Example 3: Screening of Parameter Combination n(NaIO₄)*V*C(MeCN)*T

According to RP 1, RuO₂.xH₂O and NaIO₄ were suspended. 1 was added and the reaction was stirred at the given temperature for 0.5 h.

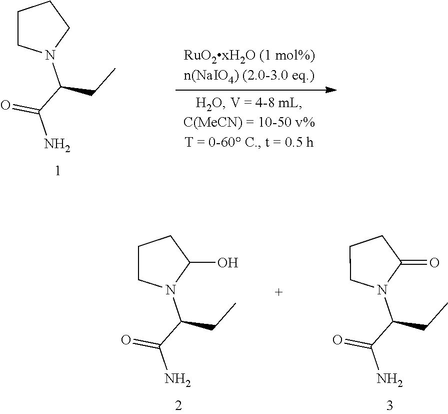

The results are summarized in Table 5.

TABLE 5

| Exp. | n(NaIO₄) | V | C(MeCN) | T | GC Yield [%] | | | |
|---|---|---|---|---|---|---|---|---|
| No. | [eq.] | [mL] | [v %] | [° C.] | 1 | 2^{a} | 3 | Σ |
| 1 | 3 | 6 | 10 | 60 | 2 | 3 | 22 | 27 |
| 2 | 3 | 4 | 10 | 60 | 6 | 4 | 24 | 34 |
| 3 | 2.5 | 4 | 10 | 0 | 24 | 8 | 37 | 69 |
| 4 | 3 | 8 | 30 | 60 | 11 | 8 | 16 | 35 |
| 5 | 3 | 6 | 50 | 0 | 1 | 2 | 65 | 68 |
| 6 | 2 | 4 | 50 | 60 | 13 | 18 | 26 | 57 |
| 7 | 2 | 4 | 30 | 0 | 17 | 7 | 51 | 75 |
| 8 | 2.5 | 8 | 50 | 60 | 8 | 17 | 18 | 43 |
| 9 | 3 | 4 | 50 | 30 | 0 | 0 | 56 | 56 |
| 10 | 3 | 8 | 10 | 0 | 1 | 4 | 35 | 40 |
| 11 | 2 | 8 | 50 | 0 | 26 | 9 | 37 | 72 |
| 12 | 2 | 8 | 10 | 30 | 6 | 19 | 18 | 43 |
| 13 | 2 | 6 | 10 | 60 | 4 | 21 | 9 | 34 |
| 14 | 2.5 | 6 | 30 | 30 | 1 | 2 | 48 | 51 |

^{a}relative area vs. standard

### Screening Example 4: Screening of Parameter Combination n(NaIO₄)*V*C(MeCN)

According to RP 1, RuO₂.xH₂O and NaIO₄ were suspended. 1 was added and the reaction was stirred at the given temperature for 0.5 h.

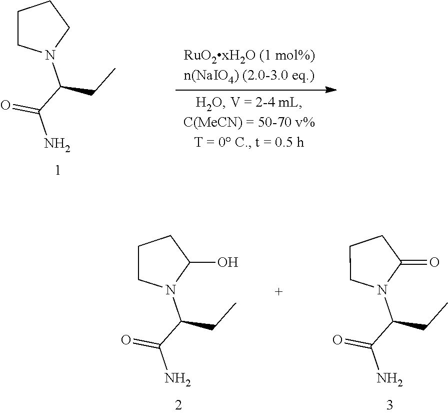

The results are summarized in Table 6.

TABLE 6

| Exp. | n(NaIO₄) | V | C(MeCN) | T | GC Yield [%] | | | |
|---|---|---|---|---|---|---|---|---|
| No. | [eq.] | [mL] | [v %] | [° C.] | 1 | 2^{a} | 3 | Σ |
| 1 | 3 | 4 | 70 | 0 | 0 | 1 | 65 | 66 |
| 2 | 3 | 2 | 70 | 0 | 0 | 0 | 66 | 66 |
| 3 | 2 | 4 | 50 | −20 | 39 | 5 | 32 | 76 |
| 4 | 2 | 2 | 50 | 0 | 13 | 3 | 51 | 67 |
| 5 | 3 | 4 | 50 | 0 | 1 | 1 | 64 | 66 |
| 6 | 3 | 2 | 50 | 0 | 0 | 0 | 66 | 66 |
| 7 | 2 | 4 | 70 | 0 | 18 | 0 | 46 | 66 |
| 8 | 2 | 4 | 50 | 0 | 43 | 6 | 25 | 74 |
| 9 | 2 | 2 | 70 | 0 | 20 | 3 | 47 | 70 |

^{a}relative area vs. standard.

### Screening Example 5: Screening of Parameter Combination n(NaIO₄)*t*C(MeCN)

According to RP 1, RuO₂.xH₂O and NaIO₄ were suspended. 1 was added and the reaction was stirred at 0° C. for the given time.

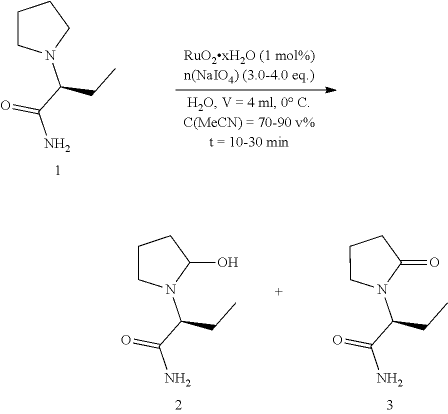

The results are summarized in Table 7.

TABLE 7

| Exp. | n(NaIO₄) | C(MeCN) | t | GC Yield [%] | | | |
|---|---|---|---|---|---|---|---|
| No. | [eq.] | [v %] | [min] | 1 | 2^{a} | 3 | Σ |
| 1 | 4 | 90 | 30 | 2 | 3 | 15 | 20 |
| 2 | 3 | 70 | 10 | 31 | 6 | 9 | 46 |
| 3 | 4 | 70 | 30 | 30 | 3 | 16 | 19 |
| 4 | 3 | 70 | 30 | 0 | 2 | 59 | 61 |
| 5 | 4 | 70 | 10 | 48 | 7 | 8 | 63 |
| 6 | 4 | 90 | 10 | 17 | 6 | 11 | 34 |
| 7 | 3 | 90 | 30 | 0 | 5 | 16 | 21 |
| 8 | 3 | 90 | 10 | 2 | 9 | 5 | 16 |
| 9 | 3.5 | 80 | 20 | 2 | 8 | 6 | 16 |

^{a}relative area vs. standard.

4.3 Screening Examples for the Synthesis of Levetiracetam with Immobilized Ruthenium Catalyst
4.3.1 RuO₂.xH₂O Immobilization on C18 Reversed Phase Material

### Screening Example 6: OVAT Experiments

The column was prepared and the experiments were carried out as according to RP 2.

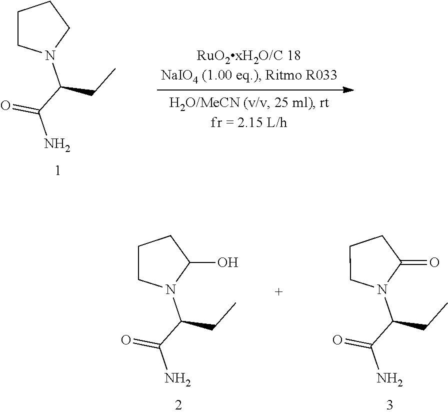

The results are summarized in Table 8.

TABLE 8

| Exp. | C(1) | C(NaIO₄) | C(MeCN) | V | | GC Yield [%] | | | |
|---|---|---|---|---|---|---|---|---|---|
| No. | [mM] | [mM] | [v %] | [mL] | leaching | 1 | 2^{a} | 3 | Σ |
| 1 | 0 | 0 | 0 | 50 | no | — | — | — | — |
| 2 | 0 | 374 | 0 | 50 | yes | — | — | — | — |
| 3 | 25.6 | 25.6 | 0 | 50 | no | 16 | 21 | 2 | 39 |
| 4 | 0 | 0 | 100 | 25 | no | p | p | 0 | 0 |
| 5 | 25.6 | 25.6 | 33% | 25 | yes | 38 | 13 | 9 | 60 |

^{a}relative area vs. standard.

### Screening Example 7: Screening of Parameter Combination C(MeCN)*C(NaIO₄)*fr

The column was prepared and the experiments were carried out as according to RP 2.

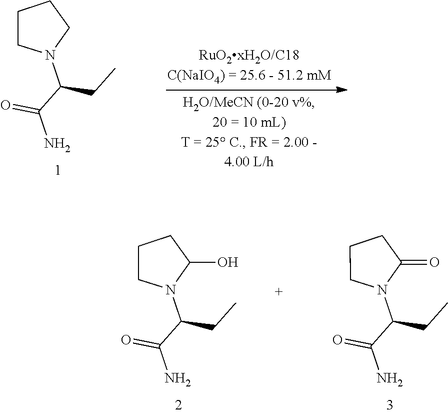

The results are summarized in Table 9.

TABLE 9

| Exp. | C(MeCN) | C(NaIO₄) | fr | | GC Yield [%] | | | |
|---|---|---|---|---|---|---|---|---|
| No. | [v %] | [mM] | [L h⁻¹]^{b} | leaching | 1 | 2^{a} | 3 | Σ |
| 1 | 20 | 25.6 | 2 | yes | 32 | 11 | 14 | 57 |
| 2 | 20 | 51.2 | 2 | yes | 31 | 16 | 17 | 64 |
| 3 | 0 | 38.4 | 2 | yes | 12 | 8 | 4 | 24 |
| 4 | 0 | 51.2 | 2 | no | 38 | 13 | 15 | 66 |
| 5 | 20 | 38.4 | 4 | yes | 46 | 22 | 22 | 90 |
| 6 | 0 | 51.2 | 4 | no | 21 | 10 | 2 | 33 |

^{a}relative area vs. standard.
^{b}Flow rate

4.3.2 RuO₂.xH₂O Immobilization on C18/PAN/Alox

### Screening Example 8: OVAT Experiments

The column was prepared and the experiment was carried out as according to RP 2.

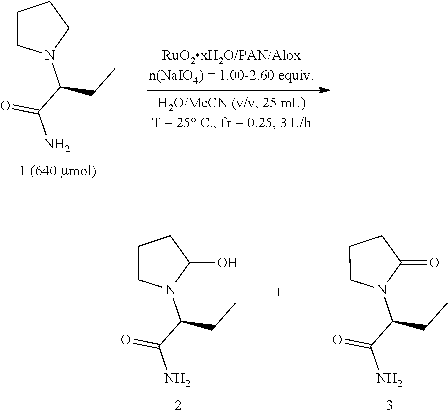

The results are summarized in Table 10.

TABLE 10

| Exp. | | fr | n(NaIO₄) | H₂O/ | GC Yield [%] | | | |
|---|---|---|---|---|---|---|---|---|
| No. | flow mode | [L/h] | [eq.] | MeCN | 1 | 2^{a} | 3 | Σ |
| 1 | single pass | 3 | 1.00 | pure H₂O | 76 | 20 | 9 | 105 |
| 2 | single pass | 0.25 | 2.60 | pure H₂O | 56 | 14 | 6 | 76 |
| 3 | cyclic, | 0.25 | 1.00 | pure H₂O | 36 | 19 | 15 | 70 |
| | 30 min | | | | | | | |
| 4 | single pass | 0.25 | 2.60 | 2:1 | 21 | 24 | 15 | 60 |
| 5 | cyclic, | 0.25 | 2.60 | pure H₂O | 4 | 19 | 20 | 43 |
| | 30 min | | | | | | | |
| 6 | cyclic, | 0.25 | 1.00 | 2:1 | 44 | 23 | 8 | 75 |
| | 30 min | | | | | | | |

^{a}relative area vs. standard.

4.3.3 RuO₂.xH₂O Immobilization on Aluminum Oxide

### Screening Example 9: OVAT Experiments

The column was prepared and the experiments were carried out as according to RP 2.

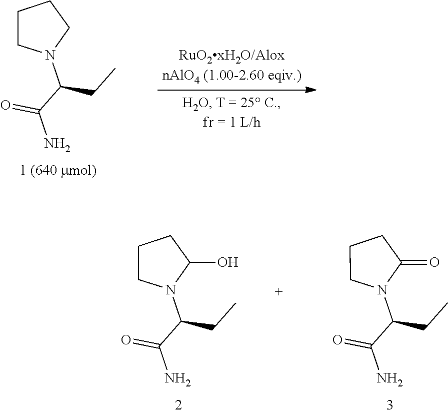

The results are summarized in Table 11.

TABLE 11

| Exp. | n(NaIO₄) | | GC Yield [%] | | | |
|---|---|---|---|---|---|---|
| No. | [eq.] | Notes | 1 | 2^{a} | 3 | Σ |
| 1 | 2.60 | clear soln. | 25 | 14 | 6 | 45 |
| 2 | 2.00 | particles in wash soln. | 42 | 12 | 7 | 61 |
| 3 | 1.00 | particles in reaction | 48 | 11 | 4 | 63 |

4.3.4 RuO₂.xH₂O Immobilization on Charcoal

### Screening Example 10: OVAT Experiments

According to RP 2, 1 was electrolyzed in caustic soda.

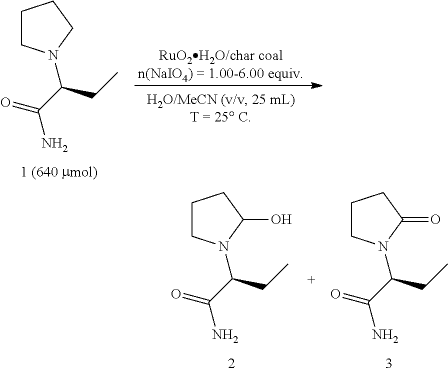

The results are summarized in Table 12.

TABLE 12

| | | H₂O/ | | | | | |
|---|---|---|---|---|---|---|---|
| Exp. | n(NaIO₄) | MeCN | | GC Yield [%] | | | |
| No. | [eq.] | [v/v] | leaching | 1 | 2^{a} | 3 | Σ |
| 1 | 2.60 | pure H₂O | no | 60 | 4 | 0 | 64 |
| 2 | 2.00 | pure H₂O | no | 50 | 6 | 0 | 56 |
| 3 | 1.00 | pure H₂O | no | 42 | 6 | 2 | 50 |
| 4 | 2.60 | 2:1 | no | 70 | 8 | 8 | 86 |
| 5 | 2.00 | 2:1 | no | 43 | 7 | 6 | 56 |
| 6 | 1.00 | 2:1 | no | 37 | 5 | 6 | 48 |
| 7 | 4.00 | 4:1 | no | 32 | 9 | 8 | 49 |
| 8 | 5.00 | 4:1 | yellow | 21 | 7 | 8 | 36 |
| | | | solution | | | | |
| 9 | 6.00 | 4:1 | no | 12 | 4 | 19 | 35 |
| 10 | 6.00 | 4:1 | no | 13 | 4 | 20 | 37 |
| 11 | 6.00 | 4:1 | no | 10 | 3 | 15 | 28 |
| 12 | 6.00 | 4:1 | no | 10 | 4 | 12 | 25 |

### 4.4 Screening Examples for the Electrochemical Synthesis of Etiracetam

### Screening Example 11: Anode Material

According to RP 3, 1 was electrolyzed in caustic soda.

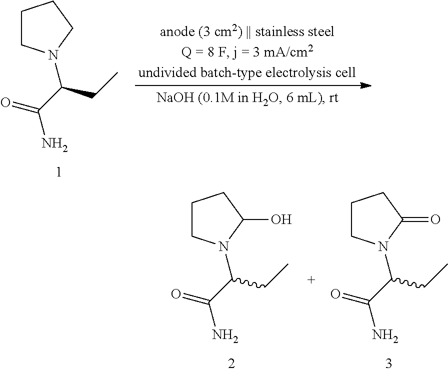

The results are summarized in Table 13.

TABLE 13

GC Yield [%]

| Exp. No. | Anode | 1 | 2^{a} | 3 | Σ |
|---|---|---|---|---|---|
| 1 | GC | 0 | 0 | 6 | 6 |
| 2 | Graphite | 0 | 0 | 7 | 7 |
| 3 | BDD | 0 | 0 | <1 | <1 |
| 4 | Carbon fiber | 0 | 0 | 3 | 3 |
| 5 | Ni | 41 | 20 | 0 | 61 |
| 6 | Pt | 0 | 0 | 15 | 15 |
| 7 | Pt | 0 | 0 | 2 | 2 |
| 8 | Pt | 0 | 0 | 2 | 2 |
| 9 | Cu | 3 | 33 | 2 | 38 |
| 10 | Au | 0 | 20 | 6 | 26 |
| 11 | SS | n.d. | — | — | — |
| 12 | Nb | n.d. | — | — | — |
| 13 | Mo | n.d. | — | — | — |
| 14 | W | n.d. | — | — | — |
| 15 | Pt | 0 | 0 | 22 | 22 |
| 16 | BDD | 0 | 0 | 9 | 9 |

^{a}relative area vs. standard.

Screening Example 12: Applied Charge

According to RP 3, 1 was electrolyzed in caustic soda.

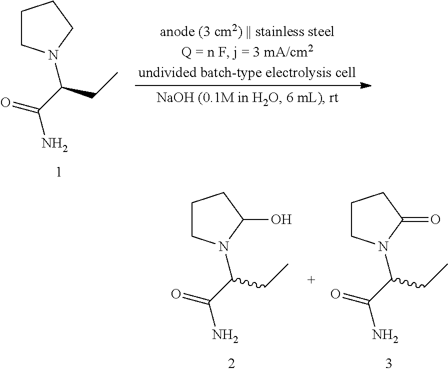

The results are summarized in Table 14.

TABLE 14

| | | |
|---|---|---|
| Exp. | Q | GC Yield [%] |

| No. | Anode | [F] | 1 | 2^{a} | 3 | Σ |
|---|---|---|---|---|---|---|
| 1 | Pt | 4.0 | <1 | 32 | 8 | 41 |
| 2 | Pt | 5.0 | 0 | 19 | 15 | 34 |
| 3 | Pt | 6.0 | 0 | 0 | 22 | 22 |
| 4 | Pt | 7.0 | 0 | 0 | 18 | 18 |
| 5 | Pt | 8.0 | 0 | 0 | 15 | 15 |
| 6 | BDD | 4.0 | 4 | 42 | 4 | 50 |
| 7 | BDD | 5.0 | 0 | 0 | 25 | 25 |
| 8 | BDD | 6.0 | 0 | 0 | 21 | 21 |
| 9 | BDD | 7.0 | 0 | 0 | 9 | 9 |
| 10 | BDD | 8.0 | 0 | 0 | 9 | 9 |
| 11 | GC | 4.0 | 0 | 36 | 6 | 42 |
| 12 | GC | 5.0 | 0 | 12 | 13 | 25 |
| 13 | GC | 6.0 | 5 | 0 | 10 | 15 |
| 14 | GC | 7.0 | 0 | 0 | 9 | 9 |
| 15 | GC | 8.0 | 0 | 0 | 6 | 6 |

^{a}relative area vs. standard.

### Screening Example 13: Current Density

According to RP 3, 1 was electrolyzed in caustic soda.

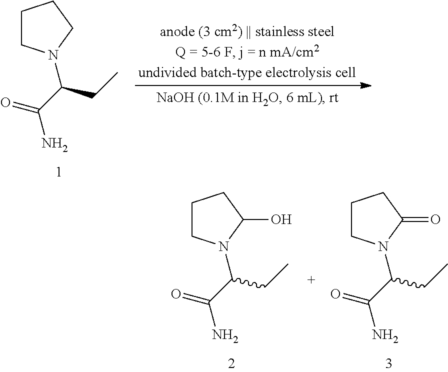

The results are summarized in Table 15.

TABLE 15

| Exp. | | Q | j | GC Yield [%] | | | |
|---|---|---|---|---|---|---|---|
| No. | Anode | [F] | [mA/cm²] | 1 | 2^{a} | 3 | Σ |
| 1 | Pt | 6.0 F | 0.5 | 0 | 0 | 10 | 10 |
| 2 | Pt | 6.0 F | 1 | 1 | <1 | 21 | 23 |
| 3 | Pt | 6.0 F | 2 | 0 | 0 | 28 | 28 |
| 4 | Pt | 6.0 F | 3 | 0 | 0 | 22 | 22 |
| 5 | Pt | 6.0 F | 5 | 0 | 0 | 26 | 26 |
| 6 | Pt | 6.0 F | 8 | 0 | <1 | 21 | 22 |
| 7 | Pt | 8.0 F | 10 | 0 | 0 | 7 | 7 |
| 8 | Pt | 6.0 F | 10 | 0 | <1 | 20 | 21 |
| 9 | Pt | 6.0 F | 12 | 0 | 0 | 20 | 20 |
| 10 | Pt | 6.0 F | 15 | 0 | 1 | 18 | 19 |
| 11 | Pt | 6.0 F | 18 | 0 | 2 | 17 | 19 |
| 12 | Pt | 8.0 F | 20 | <1 | 1 | 7 | 9 |
| 13 | Pt | 6.0 F | 20 | 0 | 1 | 15 | 16 |
| 14 | Pt | 6.0 F | 30 | 2 | 4 | 14 | 20 |
| 15 | Pt | 6.0 F | 50 | 9 | 5 | 10 | 24 |
| 16 | Pt | 6.0 F | 500 | 55 | 7 | <1 | 63 |
| 17 | BDD | 5.0 F | 0.5 | 0 | 0 | 18 | 18 |
| 18 | BDD | 5.0 F | 1 | 4 | 7 | 22 | 33 |
| 19 | BDD | 5.0 F | 2 | 0 | <1 | 28 | 29 |
| 20 | BDD | 5.0 F | 3 | 0 | 0 | 25 | 25 |
| 21 | BDD | 5.0 F | 5 | 5 | <1 | 22 | 28 |
| 22 | BDD | 5.0 F | 10 | 0 | 2 | 20 | 22 |
| 23 | BDD | 5.0 F | 15 | <1 | 2 | 17 | 20 |
| 24 | BDD | 5.0 F | 20 | <1 | 4 | 17 | 22 |
| 25 | BDD | 5.0 F | 30 | 1 | 5 | 15 | 21 |
| 26 | Pt/Si | 6.0 F | 30 | 6 | 3 | 16 | 25 |

^{a}relative area vs. standard.

### Screening Example 14: Base

According to RP 3, 1 was electrolyzed in alkaline solution.

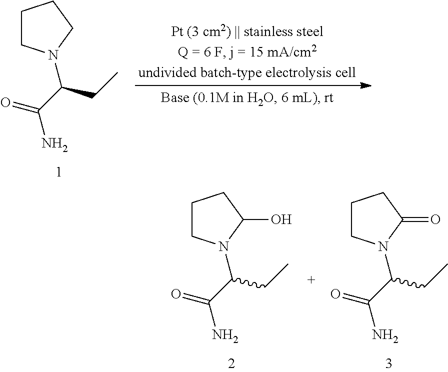

The results are summarized in Table 16.

TABLE 16

GC Yield [%]

| Exp. No. | Anode | Base | 1 | 2^{a} | 3 | Σ |
|---|---|---|---|---|---|---|
| 1 | Pt | K₂CO₃ | <1 | <1 | 6 | 8 |
| 2 | Pt | LiOH | <1 | 2 | 16 | 19 |
| 3 | Pt | NaOH | 0 | 1 | 21 | 22 |
| 4 | Pt | KOH | 7 | 4 | 10 | 21 |
| 5 | Pt | Ba(OH)₂ | 32 | 15 | 8 | 55 |
| 6 | Pt | CsOH | <1 | <1 | 5 | 7 |

^{a}relative area vs. standard.

### Screening Example 15: Base Concentration

According to RP 3, 1 was electrolyzed in caustic soda.

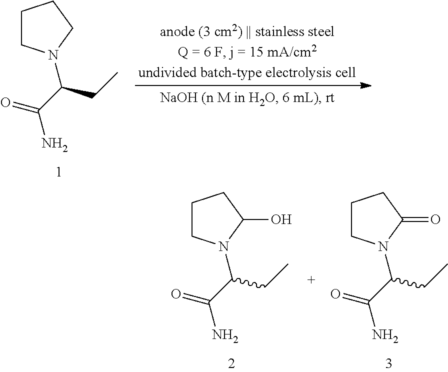

The results are summarized in Table 17.

TABLE 17

| | |
|---|---|
| C(NaOH) | GC Yield [%] |

| Exp. No. | Anode | [□] | 1 | 2^{a} | 3 | Σ |
|---|---|---|---|---|---|---|
| 1 | Pt | 0 | 28 | 3 | <1 | 32 |
| 2 | Pt | 0.01 | 38 | 8 | 2 | 48 |
| 3 | Pt | 0.05 | 15 | <1 | 5 | 21 |
| 4 | Pt | 0.07 | <1 | <1 | 13 | 15 |
| 5 | Pt | 0.09 | <1 | 12 | 16 | 29 |
| 6 | Pt | 0.1 | 0 | 1 | 21 | 22 |
| 7 | Pt | 0.12 | <1 | 2 | 17 | 20 |
| 8 | Pt | 0.15 | 4 | 4 | 11 | 19 |
| 9 | Pt | 0.2 | <1 | 2 | 4 | 7 |
| 10 | Pt | 0.50 | 0 | 2 | <1 | 3 |
| 11 | Pt | 1.00 | 0 | 3 | <1 | 4 |
| 12 | BDD | 0 | 38 | 3 | <1 | 42 |
| 13 | BDD | 0.05 | <1 | 6 | 10 | 17 |
| 14 | BDD | 0.07 | 0 | 4 | 16 | 20 |
| 15 | BDD | 0.09 | <1 | 3 | 17 | 21 |
| 16 | BDD | 0.1 | <1 | 2 | 18 | 21 |

^{a}relative area vs. standard.

### Screening Example 16: Starting Material Concentration

Carried out according to general procedure RP3.

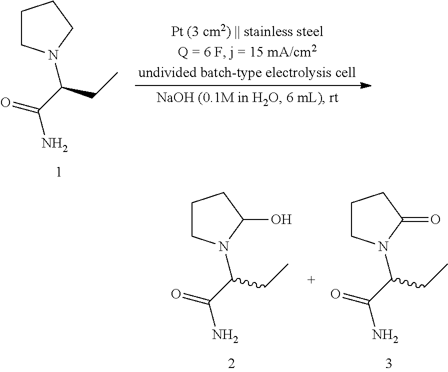

The results are summarized in Table 18.

TABLE 18

GC Yield [%]

| Exp. No. | m(1) | | 1 | 2^{a} | 3 | Σ |
|---|---|---|---|---|---|---|
| 1 | 5 | mg | 43 | <1 | 7 | 51 |
| 2 | 20 | mg | 11 | 3 | 6 | 20 |
| 3 | 40 | mg | 1 | <1 | 8 | 10 |
| 4 | 60 | mg | <1 | 1 | 13 | 15 |
| 5 | 80 | mg | 0 | <1 | 18 | 19 |
| 6 | 100 | mg | 0 | 1 | 21 | 22 |
| 7 | 150 | mg | 6 | 0 | 14 | 20 |
| 8 | 200 | mg | 7 | 0 | 10 | 17 |

^{a}relative area vs. standard.

### Screening Example 17: Stirring Velocity

According to RP 3, 1 was electrolyzed in caustic soda.

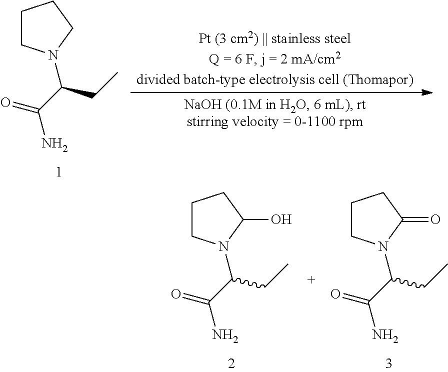

The results are summarized in Table 19.

TABLE 19

| | |
|---|---|
| stirring velocity | GC Yield [%] |

| Exp. No. | [rpm] | 1 | 2^{a} | 3 | Σ |
|---|---|---|---|---|---|
| 1 | 1100 | 0 | 0 | 27 | 27 |
| 2 | 900 | <1 | 0 | 26 | 27 |
| 3 | 250 | <1 | 0 | 24 | 25 |
| 4 | 100 | 0 | 0 | 25 | 25 |
| 5 | 0 | 3 | 5 | 11 | 19 |

^{a}relative area vs. standard.

### Screening Example 18: Temperature

According to RP 3, 1 was electrolyzed in caustic soda.

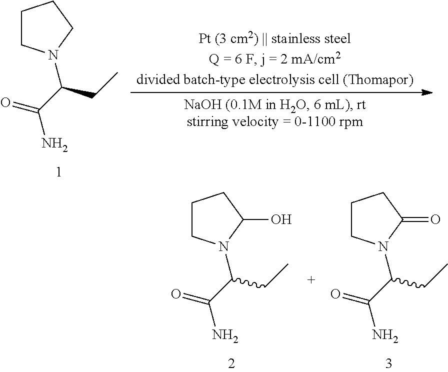

The results are summarized in Table 20.

TABLE 20

GC Yield [%]

| Exp. No. | T [° C.] | 1 | 2^{a} | 3 | Σ |
|---|---|---|---|---|---|
| 1 | 75-80 | <1 | <1 | 19 | 21 |
| 2 | 60 | 0 | 0 | 25 | 25 |
| 3 | 40 | 0 | 0 | 29 | 29 |
| 4 | Rt | <1 | 0 | 24 | 25 |
| 5 | 0-rt | 0 | 0 | 20 | 20 |

^{a}relative area vs. standard.

### 4.5 Screening Examples for Electrochemical NaIO₃ Recycling

### Screening Example 19: Screening of Parameter Combination j*Q*C(NaOH)*C(NaIO3

According to RP 4, sodium iodate was electrolyzed in caustic soda at a BDD anode.

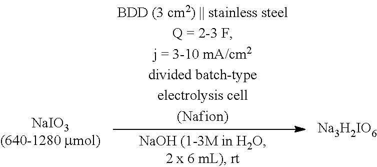

The results are summarized in Table 21.

TABLE 21

| | j | Q | C(NaOH) | n(NaIO₃) | LC-PDA Yield [%] | | |
|---|---|---|---|---|---|---|---|
| Exp. No. | [mA/cm²] | [F] | [M] | [μmol] | NaIO₃ | NaIO₄ | Σ |
| 1 | 2 | 3 | 3 | 640 | 96 | 4 | 100 |
| 2 | 2 | 2 | 2 | 640 | 62 | 38 | 100 |
| 3 | 2 | 1 | 3 | 1280 | 81 | 10 | 91 |
| 4 | 2 | 1 | 3 | 1280 | 77 | 21 | 98 |
| 5 | 2 | 2 | 3 | 1280 | 52 | 35 | 87 |
| 6 | 6 | 1.25 | 2 | 960 | 39 | 62 | 101 |
| 7 | 6 | 2.5 | 2 | 960 | 45 | 51 | 96 |
| 8 | 2 | 2 | 1 | 640 | 60 | 39 | 99 |
| 9 | 10 | 1.5 | 3 | 1280 | 102 | 2 | 104 |
| 10 | 10 | 3 | 3 | 1280 | 34 | 50 | 84 |
| 11 | 2 | 3 | 3 | 640 | 66 | 30 | 96 |
| 12 | 10 | 1 | 1 | 1280 | 44 | 62 | 96 |
| 13 | 10 | 2 | 1 | 1280 | 28 | 66 | 96 |
| 14 | 6 | 1.25 | 2 | 960 | 44 | 54 | 98 |
| 15 | 6 | 2.5 | 2 | 960 | 96 | 3 | 99 |
| 16 | 10 | 1 | 1 | 1280 | 98 | 3 | 101 |
| 17 | 10 | 2 | 1 | 1280 | 66 | 26 | 92 |
| 18 | 2 | 1.5 | 1 | 1280 | 10 | 19 | 29 |
| 19 | 2 | 1.5 | 1 | 1280 | 61 | 20 | 81 |
| 20 | 2 | 3 | 1 | 1280 | 37 | 33 | 70 |
| 21 | 10 | 3 | 1 | 640 | 93 | 5 | 98 |
| 22 | 10 | 2 | 3 | 640 | 30 | 69 | 99 |
| 23 | 2 | 1.5 | 1 | 1280 | 36 | 2 | 38 |
| 24 | 2 | 1.5 | 1 | 1280 | 41 | 53 | 94 |
| 25 | 2 | 3 | 1 | 1280 | 27 | 24 | 51 |
| 26 | 10 | 2 | 3 | 640 | 55 | 38 | 93 |
| 27 | 2 | 1 | 3 | 1280 | 22 | 8 | 30 |
| 28 | 2 | 1 | 3 | 1280 | 92 | 3 | 95 |
| 29 | 2 | 2 | 3 | 1280 | 69 | 13 | 82 |
| 30 | 10 | 3 | 1 | 640 | 44 | 34 | 77 |
| 31 | 10 | 3 | 1 | 640 | 83 | 8 | 91 |
| 32 | 10 | 1.5 | 3 | 1280 | 41 | 22 | 63 |
| 33 | 10 | 1.5 | 3 | 1280 | 51 | 28 | 79 |
| 34 | 10 | 3 | 3 | 1280 | 32 | 2 | 34 |
| 35 | 2 | 3 | 3 | 640 | 57 | 29 | 86 |
| 36 | 2 | 2 | 1 | 640 | 38 | 50 | 88 |
| 37 | 2 | 2 | 3 | 1280 | 80 | 4 | 84 |
| 38 | 6 | 2.5 | 2 | 960 | 69 | 32 | 101 |
| 39 | 2 | 2 | 1 | 640 | 77 | 22 | 99 |
| 40 | 10 | 3 | 3 | 1280 | 88 | 15 | 103 |
| 41 | 2 | 3 | 3 | 640 | 20 | 83 | 103 |
| 42 | 10 | 2 | 1 | 1280 | 94 | 3 | 97 |
| 43 | 6 | 2.5 | 2 | 960 | 92 | 3 | 95 |
| 44 | 10 | 2 | 1 | 1280 | 88 | 3 | 91 |
| 45 | 2 | 3 | 1 | 1280 | 90 | 6 | 96 |
| 46 | 10 | 3 | 1 | 640 | 77 | 7 | 84 |
| 47 | 10 | 2 | 3 | 640 | 62 | 24 | 86 |
| 48 | 2 | 3 | 1 | 1280 | 47 | 51 | 98 |
| 49 | 10 | 2 | 3 | 640 | 61 | 31 | 92 |
| 50 | 2 | 2 | 3 | 1280 | 89 | 4 | 93 |
| 51 | 10 | 3 | 1 | 640 | 36 | 54 | 90 |
| 52 | 10 | 3 | 3 | 1280 | 51 | 44 | 95 |
| 53 | 10 | 3 | 1 | 1280 | 21 | 86 | 107 |

### Screening Example 20: Screening of Parameter Combination j*C(NaOH)

According to RP 4, sodium iodate was electrolyzed in caustic soda at a BDD anode.

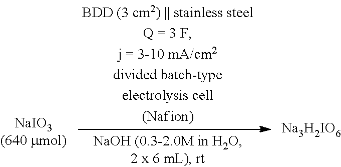

The results are summarized in Table 22.

TABLE 22

| | | | LC-PDA Yield [%] | | |
|---|---|---|---|---|---|
| Exp. No. | j[mA/cm²] | C(NaOH) [M] | NaIO₃ | NaIO₄ | Σ |
| 1 | 300 | 1.0 | 94 | 7 | 101 |
| 2 | 100 | 0.3 | 87 | 13 | 100 |
| 3 | 300 | 1.0 | 100 | 2 | 103 |
| 4 | 100 | 1.0 | 78 | 21 | 99 |
| 5 | 300 | 0.3 | 92 | 8 | 100 |
| 6 | 100 | 0.3 | 93 | 5 | 98 |
| 7 | 100 | 1.0 | 96 | 2 | 98 |
| 8 | 300 | 0.3 | 98 | 4 | 102 |
| 9 | 200 | 0.65 | 91 | 2 | 93 |
| 10 | 100 | 1.0 | 82 | 16 | 99 |
| 11 | 100 | 2.0 | 98 | 4 | 102 |
| 12 | 10 | 1.0 | 16 | 83 | 98 |
| 13 | 100 | 2.0 | 69 | 28 | 96 |
| 14 | 55 | 1.5 | 68 | 29 | 97 |
| 15 | 100 | 1.0 | 78 | 21 | 99 |
| 16 | 10 | 2.0 | 95 | 4 | 99 |
| 17 | 10 | 2.0 | 98 | 3 | 101 |
| 18 | 10 | 1.0 | 65 | 32 | 96 |

### Screening Example 21: Base-Concentration Screening

According to RP 4, sodium iodate was electrolyzed in caustic soda at a BDD anode

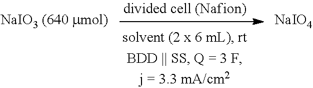

The results are summarized in Table 23.

TABLE 23

| | | | LC-PDA Yield [%] | | |
|---|---|---|---|---|---|
| Exp. No. | C(NaOH) [M] | j[mA/cm²] | NaIO₃ | NaIO₄ | Σ |
| 1 | 0.0 | 3 | 94 | 2 | 96 |
| 2 | 0.1 | 3 | 74 | 23 | 97 |
| 3 | 0.1 | 10 | 99 | 7 | 106 |
| 4 | 0.3 | 10 | 103 | 4 | 106 |
| 5 | 0.5 | 10 | 50 | 25 | 75 |
| 6 | 0.8 | 10 | 30 | 69 | 99 |
| 7 | 0.9 | 10 | 19 | 80 | 99 |
| 8 | 1.0 | 3 | 11 | 86 | 97 |
| 9 | 1.1 | 10 | 22 | 75 | 97 |
| 10 | 1.2 | 10 | 97 | 3 | 100 |
| 11 | 1.3 | 10 | 27 | 70 | 97 |
| 12 | 1.4 | 10 | 97 | 4 | 101 |
| 13 | 1.5 | 10 | 23 | 77 | 100 |
| 14 | 2.0 | 3 | 73 | 24 | 97 |
| 15 | 3.0 | 3 | 96 | 4 | 100 |
| 16 | 4.0 | 3 | 12 | 78 | 90 |
| 17 | 5.0 | 3 | 16 | 85 | 101 |

### 4.6 Specific Examples for the Synthesis of Levetiracetam, Etiracetam, Corresponding Starting Materials and NaIO₃ Recycling

### Reference Example 1: Synthesis of 2-(pyrrolidin-1-yl)butanenitrile (rac-4)

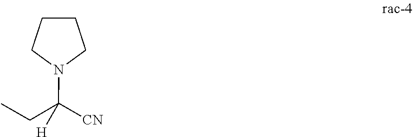

The preparation has been done according to the modified procedure of Orejarena Pacheco et al (J. C. Orejarena Pacheco, T. Opatz, The Journal of Organic Chemistry 2014, 79, 5182-5192).

Propanal (17.97 g, 22.5 mL, 309.3 mmol, 1.1 eq.) was dissolved in a water methanol mixture (2000 mL, 4:1, ˜7 mL/mmol) and NaHSO₃ (32.19 g, 309.3 mmol, 1.1 eq.) was added in one portion. The solution was stirred for 2 h and pyrrolidine (20.0 g, 23.53 mL, 281.2 mmol, 1.0 eq.) was carefully added (big batches >0.1 mol needed cooling with an ice bath). KCN (36.62 g, 562.4 mmol, 2.0 eq.) was added carefully and the mixture stirred for an additional 16 h. The reaction mixture was extracted with ethyl acetate in a Kutscher-Steudel apparatus. (F. Kutscher, H. Steudel, in Hoppe-Seyler's Zeitschrift für physiologische Chemie, Vol. 39, 1903, p. 473). The organic extract was dried over sodium sulfate, filtered and concentrated in vacuo to yield the crude product. The alpha-aminonitrile was purified by distillation (95° C., 23 mbar) to yield a colorless oil (51%-86%). The reaction was scaled from 10 mmol (711 mg 4) up to 2.0 mol (142 g 4).

Bp: 95° C. (23 mbar).

IR (ATR): ν=2970 (s), 2939 (m), 2879 (m), 2810 (m), 2222 (w), 1461 (m), 1355 (w), 1151 (m), 1085 (m), 872 (m) cm⁻¹.

¹H-NMR, COSY (Correlated Spectroscopy) (300 MHz, CDCl₃): δ=3.63 (t, ³H_{H-2, H-1}=7.8 Hz, 1H, H-1), 2.75-2.52 (m, 4H, H-2′, H-5′), 1.88-1.71 (m, 6H, H-2, H-3′, H-4′), 1.05 (t, ³J_{H-2, H-3j}=7.4 Hz, 3H, H-3).

¹³C-NMR, HMBC (Heteronuclear Multiple Bond Correlation), HSQC (Heteronuclear Single Quantum Coherence) (75 MHz, CDCl₃): δ=117.7 (CN), 57.2 (C-1), 50.1 (C-2′, C-5′), 26.2 (C-2), 23.5 (C-3′, C-4′), 10.9 (C-3).

ESI-MS: m/z (%)=139.1 (100) [C₈H₁₅N₂]⁺, 112.3 (10) [C₇H₁₄N]⁺.

### Reference Example 2: Synthesis of (S)-2-(pyrrolidin-1-yl)butanamide (S)-1

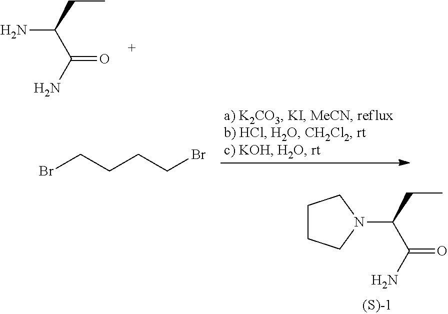

The synthesis of (S)-2-(pyrrolidin-1-yl)butanamide was carried out according to Xiaofeng et al. (Xiaofeng et al., J. Am. Chem. Soc. 2016, 138, 7872). To a 250 mL flask was added 2-amino-acetamide hydrochloride (20 mmol), 1,4-dibromobutane (24 mmol), K₂CO₃ (8.29 g, 60 mmol), Kl (332 mg, 2 mmol), and acetonitrile (80 mL). The reaction mixture was heated to reflux. After 1 d, the reaction was quenched with conc. HCl (50 mL), and water and dichloromethane were added for extraction. The organic layer was separated and discarded. The aqueous layer was made basic with potassium hydroxide solution (˜15 g in 50 mL H₂O, pH>14) and extracted with dichloromethane (4×50 mL). The organic fractions were combined, dried over anhydrous MgSO₄ and concentrated under reduce pressure to give a white fluffy solid. If necessary, the product can be recrystallized with i-PrOH at ambient temperature. Alternatively, the K₂CO₃ is filtered off after complete reaction, the solvent is removed and the solid crude product is recrystallised in acetone at 4° C. The crystals are washed with cyclohexane and dried under vacuum. The product was yielded to 67% (2.10 g, 13.5 mmol) >99% ee.

Analysis of 2-aminoacetamide hydrochloride: TLC (SiO₂, CH₂Cl₂/MeOH=1:1, KMnO₄ or bromocresol-green stain), R_{f}=0.22; ¹H NMR (400 MHz, methanol-d₄) δ 3.87 (t, J=6.3 Hz, 1H), 2.08-1.69 (m, 2H), 1.05 (t, J=7.5 Hz, 3H), ¹³C NMR (101 MHz, methanol-d₄) δ 172.33 (C_{q}), 55.27, 25.78 (CH₂), 9.36.

Analysis of 1: TLC (SiO₂, KMnO₄ or bromocresol-green stain w/o heating or ninhydrin stain with strong heating), R_{f}(CH₂Cl₂/MeOH=1:1)=0.57, R_{f}(CH₂Cl₂/MeOH=10:1)=0.22, R_{f}(CH₂Cl₂/MeOH=20:1)=0.05; GC: R_{f}=7.57 min@˜150° C.; ¹H NMR (400 MHz, CDCl₃) δ 6.65 (s, 1H), 6.00 (s, 1H), 2.69-2.62 (m, 1H), 2.62-2.50 (m, 4H), 1.89-1.57 (m, 6H), 0.94 (t, J=7.5 Hz, 3H); ¹³C NMR (101 MHz, chloroform-d) δ 176.64 (C_{q}), 71.01 (CH₂), 51.93, 25.29, 23.35, 9.97 (CH₂); LC-MS (HR ESI(+)): calculated for C₈H₁₆N₂O 156.1263 Da, found: [M+H]⁺ 157.1338 Da. Obtained spectra are in accordance with previously reported data. High solubility in H₂O (neutral and basic), MeOH, EtOH, CH₂Cl₂, CHCl₃, MeCN, low solubility in THF and 1,6-Dioxane; bₚ=282° C.

### Reference Example 3: Synthesis of Substrate 2-(pyrrolidin-1-yl)butanamide (1) from 2-(pyrrolidin-1-yl)butanenitrile (4)

The preparation was carried out according to the modified procedure of Stotani et al. (S. Stotani, C. Lorenz, M. Winkler, F. Medda, E. Picazo, R. Ortega Martinez, A. Karawajczyk, J. Sánchez-Quesada, F. Giordanetto, ACS Combinatorial Science 2016, 18, 330-336.).

The 2-(pyrrolidin-1-yl)butanenitrile (4, 10.0 g, 72.4 mmol, 1.0 eq.) was dissolved in dichlormethane (101 mL, 1.4 mL/mmol) and conc. sulfuric acid (142.0 g, 77.0 mL, 1447 mmol, 20.0 eq.) was added in one portion. The biphasic mixture was vigorously stirred for 16 h. The two layers were separated and the sulfuric acid was slowly poured into crushed ice. Under ice cooling the aqueous mixture was basified to pH 14 with cold conc. sodium hydroxide solution. The slurry was extracted with ethyl acetate in a Kutscher-Steudel apparatus (Kutscher. F. et al Hoppe-Seyler's Zeitschrift fur physiologische Chemie, Vol. 39, 1903, p. 573. The organic layer was dried over sodium sulfate and concentrated in vacuo to yield the product as a slightly yellow needles (97%-99%). The reaction was scaled from 1 mmol (138.2 mg of 4) up to 0.5 mol (69.1 g of 4).

Mp: 121.4-121.6° C.

IR (ATR): ν=3382 (m), 3186 (m), 2964 (m), 2796 (m), 1653 (vs), 1408 (m), 1332 (m), 688 (m) cm⁻¹.

¹H-NMR, COSY (300 MHz, CDCl₃): δ=6.62 (SB, 1H, NH₂), 5.87 (SB, 1H, NH₂), 2.64 (dd, ³J_{H-2a, H-1}=8.2 Hz, ³J_{H-2b, H-1}=4.3 Hz 1H, H-1), 2.61-2.49 (m, 4H, H-2′, H-5′), 1.89-1.54 (m, 6H, H-2, H-3′, H-4′), 0.94 (t, ³J_{H-2, H-3j}=7.5 Hz, 3H, H-3).

¹³C-NMR, HMBC, HSQC (75 MHz, CDCl₃): δ=176.7 (CONH₂), 71.1 (C-1), 51.9 (C-2′, C-5′), 25.3 (C-2), 23.4 (C-3′, C-4′), 10.0 (C-3).

ESI-MS: m/z (%)=157.1 (100) [C₈H₁₇N₂O]⁺, 112.2 (25) [C₇H₁₄N]⁺, 179.0 (12) [C₈H₁₆N₂ONa]⁺.

### Reference Example 4: Synthesis of 2-(2-hydroxypyrrolidin-1-yl)butanamide (2)

2 was synthesized according to RP 1. It was isolated by flash column chromatography on silica gel (12×2 cm) using an eluent mixture of CH2Cl2/MeOH=10:1; or by crystallisation in Et2O or PE/CH2Cl2 at −20° C.

¹H-NMR, COSY (300 MHz, CDCl₃): δ=7.76 (OH), 4.83 (m, 1H, H-2′), 3.13 (dt, ²J_{H-5′b, H-5′a}=10.2 Hz, ³J_{H-4′, H-5′a}=5.6 Hz, 1H, H-5′ₐ), 3.04 (ddd, ³J_{H-3a, H-2}=7.9 Hz, ³J_{H-3b, H-2}=4.3 Hz, ⁴J_{H-4, H-2}=1.4 Hz 1H, H-2), 2.67 (dt, ²J_{H-5′a, H-5′b}=10.2 Hz, ³J_{H-4′, H-5′b}=6.6 Hz, 1H, H-5′_{b}), 2.02 (dt, ²J_{H-3′b, H-3′a}=12.2 Hz, ³J_{H-4′, H-3′a}=6.2 Hz, 1H, H-3′ₐ), 1.79-1.64 (m, 4H, H-3ₐ, H-3′_{b}, H-4′), 1.63-1.46 (m, 1H, H-3_{b}), 0.98 (t, ³J_{H-3, H-4}=7.4 Hz, 3H, H-4).

¹³C-NMR, HMBC, HSQC (75 MHz, CDCl₃): δ=178.4 (C-1), 75.5 (C-2′), 69.2 (C-2), 56.6 (C-5′), 32.9 (C-3′), 26.1 (C-3), 23.9 (C-4′), 10.5 (H-4).

ESI-MS: m/z (%)=173.1 (100) [C₈H₁₇N₂O₂]⁺.

### Synthesis Example 1: Synthesis of 2-(2-oxopyrrolidin-1-yl)butane amide (3) with RuO₄

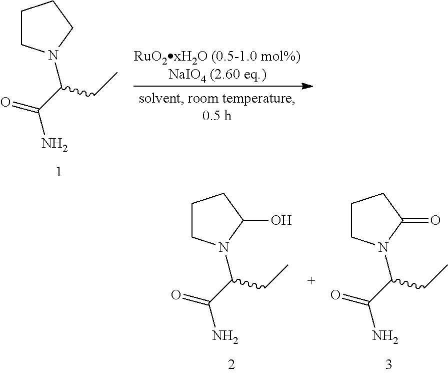

The oxidizing ruthenium(VIII) oxide was obtained in situ from RuO₂*xH₂O and NaIO₄ in a modified process.

2-(Pyrrolidin-1-yl)butane amide (1, 78.1 mg, 0.5 mmol, 1.0 eq.) was dissolved in ethyl acetate (2.5 mL) under sonification (5 min), RuO₂*xH₂O (366pg, 2.75 μmol, 0.55 mol %) and NaIO₄ solution (5 wt %, 5 mL, ≈2.6 eq.) was added. The reaction vial was closed immediately and the slurry stirred at room temperature for 30 min. The layers were separated and the aqueous layer was extracted with ethyl acetate (5×3 mL). The combined organic layers were treated with 2-propanol (2 mL) for 30 min and carefully concentrated in vacuo to yield the crude product. The product was by purified by flash column chromatography (silica gel 35-70 μm, Arcos Organics) (cyclohexane/acetic acid ethyl ester=3:1, 0.4 bar nitrogen overpressure).

2-(2-Oxopyrrolidin-1-yl)butane amide (3) was isolated with 76% yield (53.4 mg, 0.382 mmol) as colorless oil, which formed crystals.

IR (ATR): ν=3274 (m_{B}), 2969 (m), 2938 (m), 2878 (m), 1682 (vs), 1462 (m), 1422 (m), 1288 (m) cm⁻¹.

¹H-NMR, COSY (300 MHz, CDCl₃): δ=6.43 (s_{B}, 1H, NH₂), 5.75 (s_{B}, 1H, NH₂), 4.45 (dd, ³J_{H-2a, H-1}=8.9 Hz, ³J_{H-3b, H-2}=6.8 Hz 1H, H-2), 3.50-3.33 (m, 2H, H-5′), 2.47-2.36 (m, 2H, H-3′), 2.09-1.99 (m, 2H, H-4′), 2.00-1.87 (m, 1H, H-3ₐ), 1.68 (ddq, ⁴J_{H-3a, H-3b}=14.5 Hz, ³J_{H-2, H-3b}=8.9 Hz, ³J_{H-4, H-3b}=7.4 Hz, 1H, H-3_{b}), 0.89 (t, ³J_{H-3, H-4}=7.4 Hz, 3H, H-4).

¹³C-NMR, HMBC, HSQC (75 MHz, CDCl₃): δ=176.2 (C-2′), 172.5 (C-1), 56.2 (C-2), 44.0 (C-5), 31.2 (C-3′), 21.2 (C-3), 18.3 (C-4′), 10.6 (C-4).

ESI-MS: m/z (%)=193.1 (100) [C₈H₁₄N₂O₂Na]⁺, 126.1 (27) [C₇H₁₂NO]⁺.

### Synthesis Example 2: Synthesis of (S)-2-(2-oxopyrrolidin-1-yl)butane amide (3) with RuO₄

The experiment of Synthesis Example 1 was repeated with substrate 2-(pyrrolidinyl) butane amide (1) predominantly consisting of the (S)-enantiomer ((S) enantiomer 89,34%; (R) enantiomer 10.66%). The chiral HPLC (λ=210 nm; CHIRALPAK IB-3 column (250×4.6 mm, particle size 3 μm, hexane:ethanol (0.1% EDA)=90:10) of the crude product revealed a full preservation of chirality without racemization.

### Synthesis Example 3: Synthesis of (S)-2-(2-oxopyrrolidin-1-yl)butane amide (3) with RuO₄

0.5-1 mol % RuO₂.xH₂O (0.5-1.0 mg, 3.20-6.40 μmol) and 2.60 eq. of NaIO₄ (356 mg, 1.66 mmol) were suspended in acetonitrile/water (2:1) until the solution showed a pale-yellow color. (S)-1 (100 mg, 640 μmol) was added and the reaction was stirred at room temperature for 0.5 h. Levetiracetam (3) was obtained in 66% GC-yield. The product was isolated by flash column chromatography on silica gel (12×2 cm, CH₂Cl₂/MeOH=10:1). Levetiracetam was obtained in 49% isolated yield and in 99.6% ee.

TLC (SiO₂, ninhydrin stain, strong heating), R_{f}(CH₂Cl₂/MeOH=10:1)=0.56, R_{f}(CH₂Cl₂/MeOH=20:1)=0.13; GC: R_{f}=8.98 min at ˜180° C.; LC-MS (HR): calculated for C₈H₁₄N₂O₂ 170.1055 Da, found: [M+H]⁺ 171.1128; ¹H NMR (400 MHz, chloroform-d) δ 6.58 (s, 1H), 6.11-5.88 (m, 1H), 4.46 (dd, J=9.1, 6.6 Hz, 1H), 3.41 (dddd, J=34.1, 9.8, 8.0, 6.1 Hz, 2H), 2.47-2.29 (m, 2H), 2.10-1.85 (m, 3H), 1.65 (ddq, J=14.6, 9.1, 7.4 Hz, 1H), 0.86 (t, J=7.4 Hz, 3H); ¹³C NMR (101 MHz, chloroform-d) δ 176.10 (C_{q}), 172.64 (C_{q}), 56.07 (CH), 43.89 (CH₂), 31.15 (CH₂), 21.21 (CH₂), 18.20 (CH₂), 10.59 (CH₃).

### Synthesis Example 4: Synthesis of (S)-2-(2-oxopyrrolidin-1-yl)butane amide (3) with immobilized RuO₄

For the catalyst immobilization, RuO₂.xH₂O (200 mg) was mixed with aluminum oxide, C18 reversed phase material, polyacrylonitrile, charcoal, or mixtures thereof (m=25 g). The prepared material was loaded on a glass column (12×1.5 cm) and the column was connected to a Fink pump (Ritmo R033) or was alternatively pressurized using a flash adapter. For the oxidation, (S)-1 (100 mg, 640 μmol) and 2.60 eq. of NaIO₄ (356 mg, 1.66 μmol) were dissolved in water/acetonitrile (2:1 v/v, 25 mL), and the solution was pumped through the column. The system was rinsed with another 10 mL of water. The yield of levetiracetam (3) was determined by GC versus caffeine as internal standard. Levetiracetam was obtained in a maximum yield of 22%.

### Synthesis Example 5: Electrochemical Recycling of Recovered Sodium Iodate

Sodium iodate was recovered from the ruthenium-catalysis by addition of methanol to the reaction mixture. The precipitated fine crystalline needles were filtered off and were dried under reduced pressure. Iodate was isolated in up to 95% yield.

In a divided beaker cell equipped with a Nafion membrane, both chambers were filled with 6 mL of aqueous NaOH solution (1.0 M). NaIO₃ (127 mg, 640 μmol) was added to the anodic chamber and the electrolysis was started using BDD (boron-doped diamond) as anode, stainless steel as cathode, a charge amount of Q=3 F, and a current density of j=10 mA/cm². After the electrolysis was completed, the content of the anode chamber was acidified with a 1.0 M NaHSO₄ aqueous solution and analyzed by LC-PDA. Sodium periodate was obtained in 86% yield. For the isolation of the para-periodate, the precipitate was filtered off by vacuum filtration and was dried over phosphorus pentoxide under vacuum. The purity was controlled by LC-PDA and IR analysis.

The recovered sodium iodate (2.08 g, 10.5 mmol) and sodium hydroxide (2.00 g, 50.0 mmol) were dissolved in water (50 mL) and were electrolysed according to RP 4. A current density of j=50 mA cm⁻² and charge amount of Q=4 F (4055 C) were applied. Sodium paraperiodate was obtained in reproducible 83% yield as determined by LC-PDA.

For the isolation of the para-periodate, sodium hydroxide was added and the precipitate was filtered off by vacuum filtration. The solid residue was washed with water and subsequently dried in a desiccator over phosphorus pentoxide under vacuum. The conversion/purity was controlled by LC-PDA and IR analysis. The isolation of meta-periodate was carried out as according to the procedures of Mehltretter et al. and Willard et al. (H. H. Willard, R. R. Ralston, Trans. Electrochem. Soc. 1932, 62, 239; C. L. Mehltretter, C. S. Wise, U.S. Pat. No. 2,989,371A, 1961) Sodium paraperiodate (4.00 g, 13.6 mmol), HNO₃ (2.2 mL, 65%) and water (8 mL) were refluxed at 130° C. for several minutes. Water was distilled off until crystallisation started. The mixture was cooled to 4° C. and was kept at this temperature overnight. The crystals were filtered off and were dried under vacuum. Sodium meta-periodate was obtained as colorless crystals (2.057 g, 9.62 mmol, 71%). IR data were in accordance with the Bio-Rad database (Infrared spectral data were obtained from the Bio-Rad/Sadtler IR Data Collection, Bio-Rad Laboratories, Philadelphia, Pa. (US) and can be found under https://spectrabase.com. Spectrum ID (meta-periodate): 3ZPsHGmepSu).

### Synthesis Example 6: Synthesis of (S)-2-(2-oxopyrrolidin-1-yl)butane amide (3) with RuO₄ using electrochemically produced NaIO₄

According to the procedure in synthesis example 6, RuO₂.xH₂O (1 mg) and electrochemically generated NaIO₄ (550 mg, ˜4 eq.) were suspended. (S)-1 (100 mg, 640 μmol) was added and the reaction was stirred at room temperature for 0.5 h. Levetiracetam was obtained in reproducible 57% GC yield using caffeine as internal standard.

### Synthesis Example 7: Electrochemical Synthesis of Etiracetam

In a divided batch-electrolysis cell equipped with a Nafion membrane, a stainless-steel cathode, and a platinum anode, 1 (100 mg, 0.64 μmol) was electrolyzed in caustic soda (0.1 M, 6 mL) and at a temperature of 40° C. using a current density of j=15 mA/cm² and an applied charge of Q=6 F. Etiracetam was obtained in 29% GC-yield.

### Reference Example 5: Preparation of Au/Al₂O₃-Particles

The preparation of the Au/Al₂O₃-particles has been done according to the modified procedure of Jin et al (X. Jin, K. Kataoka, T. Yatabe, K. Yamaguchi, N. Mizuno, Angewandte Chemie

International Edition 2016, 55, 7212-7217).

In this experiment Al₂O₃— chromatography grade Aluminum oxide powder, ultra dry purchased from Sigma-Aldrich; or aluminum oxide 90 neutral—for column chromatography—activity 1 from Macherey-Nagel GmbH & Co. KG, Duren, Germany) was applied.

Al₂O₃ (2.0 g) was added to a solution of HAuCl₄.3H₂O (8.3 mM, 60 mL). The slurry was vigorously stirred for 2 h. The pH was quickly adjusted to 10 and stirred further for 24 h. The slurry was filtered through a frit (pore size ≥3) and the residue was washed water (5×250 mL). The solid was suspended in water (20 mL) and freeze-dried to yield the Au(OH)₃/Al₂O₃-precursor. The white powder was calcined at 400° C. for 2 h to yield the Au/Al₂O₃-particles (2.0 g) as a brow/purple powder.

Gold content of obtained particles: ˜0.25 mmol Au/mg particle=4.9% mAu/mg particle

In the following conversion experiments 50 mg particle/78 mg substrate to be converted is applied=3.1% mAu/mg substrate which corresponds to ˜2.5 mol % Au

### Synthesis Example 8: Synthesis of 2-(2-oxopyrrolidin-1-yl)butanamide (rac-3) with Au/Al₂O₃-particles

The oxidation of 2-(pyrrolidin-1-yl)butanamide (rac-1) to 2-(2-oxopyrrolidin-1-yl)butanamide (rac-3) was performed with the Au/Al₂O₃-particles and oxygen in water at higher temperatures. In general, the oxidation of 2-(pyrrolidin-1-yl)butanamide (rac-1) revealed a fast oxidation of the amine (rac-1) to an the hemi-aminal (2) of the formula and a slower second oxidation of the amine (rac-2) to the etiracetam (rac-3).

The following reaction conditions were applied: 2-(Pyrrolidin-1-yl)butanamide (1) (78 mg, 50 mmol, 1.0 eq.) was dissolved in water (4 mL) and the Au/Al₂O₃-particles (50 mg) (Reference Example 5) were added. The solution and the headspace were saturated with oxygen, the reaction vial was closed and heated. After cooling to room temperature, the crude product was isolated by extraction with ethyl acetate. The particles can be recovered by filtering the mixture through a glass frit (pore size≥3).

### Synthesis Example 9: Large scale synthesis of 2-(2-oxopyrrolidin-1-yl)butanamide (3) with RuO₂*xH₂O/NaIO₄

Ruthenium (IV) oxide x-hydrate (4.7 mg, 35.3 mmol) was placed in a flask and sodium periodate solution (5.0%, 50.0 mL) was added. The aqueous solution was covered with a solution of ethyl acetate (25.0 mL) and 2-(pyrrolidin-1-yl) butanamide (1.00 g, 6.41 mmol, 1.0 eq.). The flask was connected to a double bubble counter and stirred for 30 min.

The crude product was extracted from the aqueous solution using a Kutscher-Steudel apparatus and ethyl acetate, iso-propanol (20.0 mL) being added to the ethyl acetate in the extracted flask. The ethereal extract was dried over sodium sulfate and concentrated in vacuo. The crude product (953 mg) was purified by flash column chromatography (silica gel, cyclo-hexane/ethyl acetate=3:1) to obtain the product as a colorless solid (808 mg, 4.75 mmol, 74%).

### Experimental Section—Part B

### 1. Materials

All chemicals as used herein, except for those who were synthesized internally (as described herein), were of analytical grade and are obtained from commercial suppliers.

A stock solution of RuCl₃.H₂O (Alfa Aesar 47182, 7.9 mg) in H₂O (10 mL) was prepared daily to be used freshly (1 mL used for each reaction contained 0.79 mg RuCl₃.H2O).

2. Methods GC details:
Conditions: 150° C.-5 min-25° C./min-300° C.-5 min; T^{a} det: 300° C; T^{a} inj: 220° C.; split 50:1; flux: 1.5 mL/min; carrier: He
Column: HP-5; 5% Phenyl Methyl Siloxane; 30 m, 0.2 mm ID, 0.33 μm
5 mg/mL in MeOH

### Chiral HPLC Details:

Conditions: Heptane:EtOH (90:10), 25° C., λ=215 nm, flux: 1.0 mL/min
Column: Chiralpak ADH 250×4.6 mm, 5 μm
1 mg/mL in heptane:EtOH
   3. Examples—Oxidation Reactions with different catalyst materials

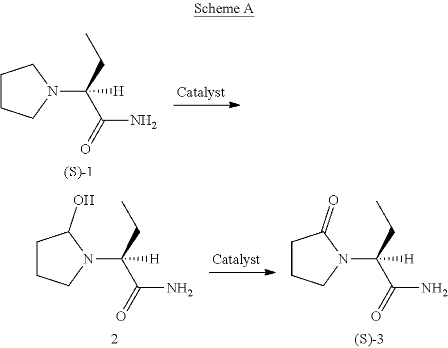

In the subsequent section experiments are described wherein it was investigated whether different heterogeneous oxidation catalyst systems are suitable for the stereospecific chemical oxidation of the pyrrolidine substrate (S)-2-(pyrrolidin-1-yl)butanamide (1) (Scheme A). The formation of the (S)- and (R)-2-(2-oxopyrrolidin-1-yl)butanamide (3) and optionally of the intermediary hemi-aminal (2) oxidation product was analyzed.

4. Oxidation Reactions with Different Catalyst Materials

### Synthesis Example 10: Synthesis of (S)-2-(2-oxopyrrolidin-1-yl)butanamide (3) with RuCl₃*xH₂O and NaIO₄

To a preformed solution of RuCl₃.H₂O in H₂O (1 mL, 0.79 mg, 3.52 μmol) was added a solution of NaIO₄ 5 wt % (278 mg, 1.3 mmol, 2.6 eq, in 5 mL H₂O). To the yellowish mixture formed, (S)-2-(pyrrolidin-1-yl)butanamide (1) (78.1 mg, 0.5 mmol) dissolved in EtOAc (2.5 mL) and H₂O (1 mL) was added. The reaction vial was vigorously stirred at room temperature for 30 minutes.

After this time, 2-propanol (2 mL) was added and the mixture was stirred for additional 30 minutes. The solid precipitated in the interphase was filtered and discarded. The aqueous layer was extracted with EtOAc, was dried with MgSO₄ and was concentrated to obtain desired product (33 mg, crude). Low recovery possibly due to presence of product in aqueous layer (as confirmed by HPLC/MS and GC).
HPLC/MS: 33% final product (3)
GC: 58% final product (3), no starting material (1)
Chiral HPLC (crude): ee 79% (S)-enantiomer of (3)

### Synthesis Example 11: Synthesis of (S)-2-(2-oxopyrrolidin-1-yl)butanamide (3) with RuCl₃*xH₂O and NaIO₄

To a preformed solution of RuCl₃.n H₂O (1 mL, 0.79 mg, 3.52 μmol) was added a solution of NaIO4 5 wt % (356 mg, 1.66 mmol, 2.6 eq, in 5 mL H₂O). To the yellowish mixture formed (S)-2-(pyrrolidin-1-yl)butanamide (1) (100 mg, 0.64 mmol) dissolved in EtOAc (2.5 mL) and H₂O (1 mL) was added. The reaction vial was vigorously stirred at room temperature for 10 minutes.

After this time, 2-propanol (2 mL) was added and the mixture was stirred for additional 30 minutes. The solid precipitated in the interphase was filtered and discarded. The aqueous layer was extracted with EtOAc, was dried with MgSO₄ and was concentrated to obtain desired product (36 mg, crude). Low recovery possibly due to presence of product in aqueous layer (as confirmed by HPLC/MS and GC).
HPLC/MS: 46% final product (3).
GC: 75% final product (3), no starting material (1).
Chiral HPLC (crude): ee 92% (S)-enantiomer of (3).

Aqueous layer was extracted with iso-butanol (x3), dried and concentrated to obtain additional 22 mg product.

### Synthesis Example 12: Synthesis of (S)-2-(2-oxopyrrolidin-1-yl)butanamide (3) with RuCl₃*xH₂O, NaIO₄ and sodium oxalate

To a preformed solution of RuCl₃ in H₂O (1 mL, 0.79 mg, 3.52 μmol) was added sodium oxalate (8.6 mg, 0.1 eq) and a solution of NaIO₄ 5 wt % (356 mg, 1.66 mmol, 2.6 eq. in 5 mL H₂O). To the yellowish mixture formed, was added (S)-2-(pyrrolidin-1-yl)butanamide (1) (100 mg, 0.64 mmol) dissolved in EtOAc (2.5 mL) and H₂O (1 mL). The reaction vial was vigorously stirred at room temperature for 10 minutes.

After this time, 2-propanol (2 mL) was added and the mixture was stirred for additional 30 minutes. The solid precipitated in the interphase was filtered and discarded. Then, the mixture was concentrated to dryness.
GC: 6% starting material (1), 7% intermediary product (2), 68% final product (3)
Chiral HPLC (crude): ee 95% (S)-enantiomer of (3)

### Synthesis Example 13: Synthesis of (S)-2-(2-oxopyrrolidin-1-yl)butanamide (3) with RuCl₃*xH₂O and NaIO₄/NaClO

To a preformed solution of RuCl₃. in H₂O (1 mL, 0.79 mg, 3.52 μmol) was added a solution of NaIO₄ 5 wt % (164 mg, 0.77 mmol; 1.2 eq. in 5 mL H₂O). To the yellowish mixture formed, was added (S)-2-(pyrrolidin-1-yl)butanamide (1) (100 mg, 0.64 mmol) dissolved in EtOAc (2.5 mL) and H₂O (1 mL). The reaction vial was vigorously stirred at room temperature for 10 minutes.
GC (1): 48% starting material (1), 17% intermediary product (2), 24% final product (3)

To the mixture was added portion wise a freshly prepared aqueous solution of NaClO (0.263 g, 1.6 mmol, 2.5 eq., in 2.5 mL H₂O) (addition of 52.6 mg/0.5 mL solution each 30 minutes).

After 2.5 h, 2-propanol (2 mL) was added, the mixture was stirred for additional 10 minutes and concentrated to dryness.
GC (2): 10% starting material (1), 31% intermediary product (2), 39% final product (3) Chiral HPLC (crude): ee 80% (S)-enantiomer of (3)

### Synthesis Example 14: Synthesis of (S)-2-(2-oxopyrrolidin-1-yl)butanamide (3) with RuCl₃*xH₂O and NaIO₄/NaClO

To a mixture of RuCl₃.in H₂O (0.79 mg, 3.52 μmol), sodium oxalate (8.58 mg, 0.064 mmol) and a solution of NaIO₄ 5 wt % (164 mg, 0.77 mmol, 1.2 eq., in 5 mL H₂O) was added (S)-2-(pyrrolidin-1-yl)butanamide (1) (100 mg, 0.64 mmol) dissolved in EtOAc (2.5 mL) and H₂O (1 mL). The reaction vial was closed and the mixture was stirred at room temperature for 10 minutes.

To the mixture was added at 0° C. a freshly prepared aqueous solution of NaClO (0.263 g, 1.6 mmol, 2.5 eq., in 2.5 mL of H₂O) portion wise (addition of 52.6 mg/0.5 mL solution each 30 minutes)

After 2 h30 min, 2-propanol (2 mL) was added, the mixture was stirred for additional 10 minutes and concentrated to dryness.
GC: 17% starting material (1), 29% intermediary product (2), 41% final product (3)
Chiral HPLC (crude): ee 92% (S)-enantiomer of (3)

The experimental details and analytical results of Synthesis Examples 7 to 11 are summarized in the following table.

The term “% eq” relates to “mol %” in the following tables.

Mol % values of applied Ru salts in the subsequent tables relate to the dehydro form of the salt.

The terms RuO₂*H₂O and RuCl₃*H₂0 refers to RuO₂*xH₂0 and RuCl₃*xH₂0, respectively, wherein x is a value in the range of 0 to 3 and above 3, taking into consideration the different stoichiometric and non-stoichiometric hydrated forms in which said salts may exist.

TABLE 24
Results of Synthesis Examples 7 to 11: Synthesis of (S)-2-(2-oxopyrrolidin-1-yl)butanamide
(3) with RuCl₃•H₂O and NaIO₄ and optionally NaClO and optionally sodium oxalate

| | | | | HPLC chiral | | | |
|---|---|---|---|---|---|---|---|
| Synthesis | | | | (crude) (S)-FP | GC results (a/a)³⁾ | | |
| Example | [Ru salt]¹⁾ | [Ox] | t | ee (%)²⁾ | SM | INT | FP |
| 10 | RuCl₃•H₂O 0.55% eq | NaIO₄ (2.6 eq) | 30′ | 79% | — | — | 58 |
| | EtOAc/H₂O (2.5:6) | | | | | | |
| 11 | RuCl₃•H₂O 0.55% eq | NaIO₄ (2.6 eq) | 10′ | 92% | — | 4 | 75 |
| | EtOAc/H₂O (2.5:6) | | | | | | |
| 12 | RuCl₃•H₂O 0.55% eq | NaIO₄ (2.6 eq) + | 10′ | 95% | 6 | 7 | 68 |
| | EtPAc/H₂O (2.5:6) | NaC₂O₄ 10% (0.1 eq) | | | | | |
| 13 | RuCl₃•H₂O 0.55% eq | 1) NaIO₄ (1.2% eq) | 1) 10′ | 80% | 48 | 17 | 24 |
| | EtOaC/H₂O (2.5:6) | 2) NaOCl•5H₂O | 2) 30′ (0.5 eq) | | 46 | 19 | 28 |
| | | (0.5 to 2.5 eq) | 1 h (1.0 eq) | | 44 | 19 | 32 |
| | | Addition 0.5 eq/30′ | 1 hr 30 (1.5 eq) | | 34 | 23 | 35 |
| | | | 2 h (2.0 eq) | | 20 | 30 | 36 |
| | | | 2 h 30 (2.5 eq) | | 10 | 31 | 39 |
| 14 | RuCl₃•H₂O 55% eq | 1) NaIO₄ (1.2 eq) | 1) 10′ | 84% | | | |
| | EtOAc/H₂O (2.5:6) | 2) NaOCl•5H₂O | 2) 30′ (0.5 eq) | | 40 | 9 | 32 |
| | | (0.5 to 2.5 eq) 0° C. | 1 h (1.0 eq) | | 33 | 17 | 38 |
| | | Addition 0.5 eq/30′ + | 1 h 30 (1.5 eq) | | 15 | 30 | 44 |
| | | NaC₂O₄ | 2 h (2.0 eq) | | 17 | 27 | 45 |
| | | | 2 h 30 (2.5 eq) | | 17 | 29 | 41 |

¹⁾preform Ru (IV) catalyst by mixing Ru salt precursor [Ru salt] and oxidant [Ox]
²⁾estimated values from crude reaction
³⁾SM = Starting material = (1); FP = Final product = (3); INT = Intermediary Product = (2)

### Synthesis Example 15: Synthesis of 2-(2-hydroxypyrrolidin-1-yl)butanamide (2) with RuCl₃H₂O and NaClO

To a solution of (S)-2-(pyrrolidin-1-yl)butanamide (1) (500 mg,3.2 mmol) in EtOAc (20 mL), ACN (20 mL) and H₂O (5 mL) was added RuCl₃.H₂O (7.21 mg, 0.032 mmol). Then, NaClO (2.63 g, 16.0 mmol) was added portion wise during 1 h.

After 1.5 h the mixture was analyzed by GC:
GC (1): 42% starting material (1), 47% intermediary product (2)

Further NaClO (2.5 equiv.) was added to complete reaction, stirring the mixture at room temperature for 30 minutes. After this time, a solution of Na₂S₂O₃ aq. sat. and MeOH was added (2 ml), stirring the mixture for 10 minutes. The reaction crude was concentrated to dryness.
GC (2): 5% starting material (1), 73% intermediary product (2), 3% final product (3)

To the crude reaction material H₂O and TBME was added. Layers were separated and aqueous layer was extracted three times with TBME, dried and concentrated to obtain 90 mg of crude intermediary product (2), which was analyzed by ¹H-NMR (MeOD) and GC (3): 81% intermediary product (2).

GC of the aqueous layer showed remaining product, so it was further extracted with i-BuOH three times to obtain further 250 mg of crude intermediary product (2).
GC (5): 6% starting material (1), 74% intermediary product (2), 4% final product (3)

### Synthesis Examples 16 to 86

In the subsequent section further Synthesis Examples 13 to 84 are described, which illustrate the oxidation of 2-(pyrrolidin-1-yl)butanamide (1) to 2-(2-hydroxypyrrolidin-1-yl)butanamide (2) and/or 2-(2-oxopyrrolidin-1-yl)butanamide (3) in the presence of different oxidation catalysts. The respective experimental conditions and results are summarized in the following Tables 25 to 33. In each of these tables, the following abbreviations apply:
SM=Starting material=(1)
FP=Final product=(3)
INT=Intermediary Product=(2)
Ox=Oxidant
Work-up (A): separate phases and add i-PrOH over organic phase
Work-up (B): add i-PrOH to the crude, filter and separate phases
Work-up (C): add Na₂S₂O₃ to the reaction mixture
Work-up (D): add Na₂S₂O₃ and MeOH to reaction mixture and concentrate to dryness

TABLE 25
Results of Synthesis Examples 16 to 19: Synthesis of (3) with RuO₂ or RuO₂*H₂O and NaIO₄

| | | | Catalyst | | | | | |
|---|---|---|---|---|---|---|---|---|
| Synthesis | | | Preparation | | GC results (a/a) | | | Work up |
| Example | [Ru precatalyst] | [Ox] | Type | t | SM | INT | FP | Type |
| 16 | RuO₂ 0.55% eq | NaIO₄ 2.6 eq. | A | 6 h | 8 | — | 54 | A |
| | EtOAc/H₂O (2.5:5) | | | | | | | |
| 17 | RuO₂ 0.55% eq | NaIO₄ 2.6 eq. | A | 30′ | 37 | 9 | 17 | A |
| | EtOAc/H₂O (2.5:6) | | | | | | | |
| 18 | RuO₂ 0.55% eq | NaIO₄ 2.6 eq. | B | 30′ | 53 | 5 | 12 | B |
| | EtOAc/H₂O (2.5:6) | | | | | | | |
| 19 | RuO₂* × H₂O 0.55% eq | NaIO₄ 2.6 eq. | B | 30′ | 3 | 15 | 67 | B |
| | EtOAc/H₂O (2.5:6) | | | | | | | |

Ex. 16: 78 mg starting material (1)
Ex. 17: 200 mg starting material (1)
Ex. 18: 100 mg starting material (1)
Catalyst preparation (A) Dissolve SM in EtOAc and add RuO₂ + oxidant
Catalyst preparation (B) Preform RuO₄ specie (mix RuO₂ + oxidant) and then add SM in EtOAc

It was observed that the hydrated precursor RuO₂H₂O provides better yields of final product (3)

TABLE 26
Results of Synthesis Examples 20 to 34: Synthesis of (2) and (3) with RuCl₃*H₂O and NaIO₄ under different conditions

| | | Catalyst | | | | | | |
|---|---|---|---|---|---|---|---|---|
| Synthesis | | Preparation | | | GC results (a/a) | | | |
| Example | [Ru Precatalyst] | Type | [Ox] | t | SM | INT | FP | Work up |
| 20 | RuCl₃*H₂O 0.55% eq | B | NaIO₄ (2.6 eq.) | 10′ | 3 | 5 | 72 | add i-PrOH and |
| | EtOAc/H₂O (2.5:6) | | | | | | | concentrate |
| 21 | RuCl₃*H₂O 0.55% eq | B | NaIO₄ (2.6 eq.) | 24 h | 0 | 6 | 76 | add i-PrOH and |
| | EtOAc/H₂O (2.5:6) | | | | | | | concentrate |
| 22 | RuCl₃*H₂O 0.55% eq | B | NaIO₄¹⁾ (2.6 eq.) | 1 h | 28 | 43 | 22²⁾ | |
| | EtOAc/H₂O (2.5:6) | | | | 0 | 24 | 52 | |
| 23 | RuCl₃*H₂O 0.55% eq | B | NaIO₄ (1.3 + 1.3 eq.) | 10′ + 10′ | 4 | 17 | 59 | add i-PrOH and |
| | EtOAc/H₂O (2.5:6) | | | | | | | concentrate |
| 24 | RuCl₃*H₂O 0.55% eq | B | NaIO₄ (2.6 eq.) + NaOAc | 10′ | 19 | — | 58 | |
| | EtOAc/H₂O (2.5:6) | | | | | | | |
| 25 | RuCl₃*H₂O 0.55% eq | B | NaIO₄ (1.8 eq.) | 10′ | 11 | 16 | 31 | add i-PrOH and |
| | EtOAc/H₂O (2.5:6) | | | | | | | concentrate |
| 26 | RuCl₃*H₂O 0.55% eq | B | NaIO₄ (1.8 eq.) | 1 h | 17 | 23 | 39 | add i-PrOH and |
| | EtOAc/H₂O (2.5:6) | | | | | | | concentrate |
| 27 | RuCl₃*H₂O 0.55% eq | B | NaIO₄ (1.2 eq.) | 10′ | 43 | 29 | 22 | add i-PrOH and |
| | EtOAc/H₂O (2.5:6) | | | | | | | concentrate |
| 28 | RuCl₃*H₂O 0.55% eq | B | NaIO₄ (1.2 eq.) | 1 h | 43 | 27 | 20 | add i-PrOH and |
| | EtOAc/H₂O (2.5:6) | | | | | | | concentrate |
| 29 | RuCl₃*H₂O 0.55% eq | B | NaIO₄ (1.2 eq.) | on | 29 | 22 | 20 | add i-PrOH and |
| | EtOAc/H₂O (2.5:6) | | | | | | | concentrate |
| 30 | RuCl₃*H₂O 1.0% eq | B | NaIO₄ (1.2 eq.) | 1 h | 41 | 26 | 22 | add i-PrOH and |
| | EtOAc/H₂O (2.5:6) | | | | | | | concentrate |
| 31 | RuCl₃*H₂O 0.55% eq | B | NaIO₄ (0.6 + 0.6 eq.) | 10′ + 10′ | 31 | 35 | 14 | add i-PrOH and |
| | EtOAc/H₂O (2.5:6) | | | | | | | concentrate |
| 32 | RuCl₃•H₂O | B | NaIO₄ (0.6 + 0.6 eq.) | 10′ + 10′ | 27 | 32 | 18 | add i-PrOH and |
| | 0.5% eq + 0.5% eq | | | | | | | concentrate |
| | EtOAc/H₂O (2.5:6) | | | | | | | |
| 33 | RuCl₃•H₂O | B | NaIO₄ (1.2 eq.) | 10′ + 10′ | 32 | 13 | 17 | add i-PrOH and |
| | 0.3% eq + 0.3% eq | | | | | | | concentrate |
| | EtOAc/H₂O (2.5:6) | | | | | | | |
| 34 | RuCl₃•H₂O 0.55% eq | B | NaIO₄ (1.2 eq.) | 1 h | 37 | 30 | 22 | add i-PrOH and |
| | EtOAc/H₂O (2.5:6) | | | 50° C. | | | | concentrate |

¹⁾slow addition of NaIO₄ (added in 4 portions during 1 h)
²⁾GC after 1.3 eq. oxidant
on = overnight

TABLE 27
Results of Synthesis Examples 35 to 42: Synthesis of (2) with RuCl₃•H₂O and NaOCl

| | [Ru | Catalyst | | | | | | |
|---|---|---|---|---|---|---|---|---|
| Synthesis | Precatalyst] | Preparation | | | GC results (a/a) | | | |
| Example | (100 mg) | Type | [Ox] | t | SM | INT | FP | Workup |
| 35 | RuCl₃*H₂O 1.0% eq | B | NaOCl*5H₂O | 1 h | 66 | 13 | 2 | D |
| | ACN/EtOAc/H₂O (4:4:0.7) | | (2.6 eq.) | | | | | |
| 36 | RuCl₃*H₂O 1.0% eq | B | NaOCl*5H₂O | 1 h | 17 | 67 | — | D |
| | ACN/EtOAc/H₂O (4:4:0.7) | | (5 eq.) | | | | | |
| 37 | RuCl₃*H₂O 1.0% eq | B | NaOCl*5H₂O | 1 h | 20 | 65 | — | D |
| | ACN/EtOAc/H₂O (4:4:0.7) | | (5 eq.) | 0° C. | | | | |
| 38 | RuCl₃*H₂O 1.0% eq | B | NaOCl*5H₂O | 10′ | 4 | 75 | — | D |
| | ACN/EtOAc/H₂O (4:4:0.7) | | (5 eq.) | | | | | |
| 39 | RuCl₃*H₂O 1.0% eq | B | NaOCl*5H₂O | 10′ | 4 | 75 | | D |
| | ACN/EtOAc/H₂O (4:4:0.7) | | (5 eq.) | | | | | |
| 40 | RuCl₃*H₂O 1.0% eq | B | NaOCl*5H₂O | 1 h | 24 | 56 | <1 | D |
| | EtOAc/H₂O (2.5:6) | | (5 eq.) | | | | | |
| 41 | RuCl₃*H₂O 1.0% eq | B | NaOCl*5H₂O | 4 h | 59 | 26 | 6 | D |
| | EtOAc/H₂O (2.5:6) | | (2.5 eq.) | | | | | |
| 42 | RuCl₃*H₂O 1.0% eq + 1.0% eq | B | NaOCl*5H₂O | 10′ + 10′ | 18 | 68 | <1 | D |
| | EtOAc/H₂O (2.5:6) | | (2.5 eq.) | | | | | |

TABLE 28
Results of Synthesis Examples 43 to 49: Effect of NaIO₄/NaClO combinations
on formation of (2) and/or (3); Recyclability of NaIO₄ with a second oxidant
(NaClO) These examples relate to Synthesis Examples 13 and 14, above.

| | | Catalyst | | | | | |
|---|---|---|---|---|---|---|---|
| Synthesis | [Ru | Preparation | | | GC results (ala) | | |
| Example | Precatalyst] | Type | [Ox] | t | SM | INT | FP |
| 27 | RuCl₃*H₂O 0.55% eq | B | NaIO₄ (1.2 eq.) | 10′ | 43 | 29 | 22 |
| (Reference) | EtOAc/H₂O (2.5:6) | | | | | | |
| 38 | RuCl₃*H₂O 1.0% eq | B | NaOCl•5H₂O (5 eq.) | 10′ | 4 | 75 | |
| (Reference) | ACN/EtOAc/H₂O (4:4:0.7) | | | | | | |
| 43 | 1) RuCl₃*H₂O 1.0% eq | B | 1) NaOCl•5H₂O (5 eq.) | 1 h | 32 | 55 | 2 |
| | ACN/EtOAc/H₂O (4:4:0.7) | | 2) NaIO₄ (1.2 eq.) | 10′ | 30 | 60 | — |
| | 2) RuCl₃*H₂O 0.55% eq | | | | | | |
| | EtOAc/H₂O (2.5:6) | | | | | | |
| 44 | RuCl₃*H₂O 0.55% eq | B | NaIO₄ (1.2 eq.) | 10′ | 13 | 33 | 25 |
| | EtOAc/H₂O (2.5:6) | | NaOCl•5H₂O (2.5 eq.) | | | | |
| 45 | RuCl₃*H₂O 0.55% eq | B | 1) NaIO₄ (1.2 eq.) | 10′ | 30 | 18 | 37 |
| | EtOAc/H₂O (2.5:6). | | 2) NaOCl•5H₂O (2.5 eq.) | | | | |
| | | | slow add | | | | |
| 46 | RuCl₃*H₂O 0.55% eq | B | 1) NaIO₄ (1.2 eq.) | 1) 10′ | 41 | 12 | 34 |
| | EtOAc/H₂O (2.5:6) | | 2) NaOCl•5H₂O (2.5 eq.) | 2) 1 h 90° C. | | | |
| 47 | RuCl₃*H₂O 0.55% eq | B | 1) NaIO₄ (1.2 eq.) | 10′ | 23 | 10 | 48 |
| | EtOAc/H₂O (2.5:6) | | 2) NaOCl•5H₂O (2.5 eq.) | 0° C. 10′ | | | |
| | | | slow add 0° C. | | | | |
| 48 | RuCl₃*H₂O 0.55% eq | B | 1) NaIO₄ (1.2 eq.) + | 1) 10′ | 33 | 22 | 23 |
| | EtOAc/H₂O (2.5:6) | | NaOAc (10%) | 2) 10′ | | | |
| | | | 2) NaOCl•5H₂O (2.5 eq.) | | | | |
| 49 | RuCl₃*H₂O 0.55% eq | B | 1) NaIO₄ (1.2 eq.) + | 1) 10′ | 6 | 35 | 30 |
| | EtOAc/H₂O (2\hat{ }6) | | sodium oxalate (10%) | 2) 10′ | | | |
| | | | 2) NaOCl•5H₂O (2.5 eq.) | | | | |

TABLE 29
Results of Synthesis Examples 50 to 56: Synthesis of Intermediary
Product (2) and Final Product (3) with RuCl₃•H₂O and Oxone

| | | Catalyst | | | | | | |
|---|---|---|---|---|---|---|---|---|
| Synthesis | [Ru Precatalyst] | Preparation | | | GC results (a/a) | | | Work up |
| Example | (100 mg) | Type | [Ox] | t | SM | INT | FP | Type |
| 50 | (B) RuCl₃*H₂O 3.5% eq | A | Oxone (1.5 eq.) | 10′ | 52 | 10 | 12 | C |
| | ACN/H₂O (7.5:4.5) | | NaHCO₃ (4.6 eq.) | | | | | |
| 51 | (B) RuCl₃*H₂O 3.5% eq | A | Oxone (1.5 eq.) | 10′ | 24 | 12 | 14 | C |
| | ACN/EtOAc/H₂O (7.5:7.5:4.5) | | NaHCO₃ (4.6 eq.) | | | | | |
| 52 | (B) RuCl₃*H₂O 1.0% eq | B | Oxone (5 eq.) | 10′ | 15 | — | 33 | C |
| | ACN/EtOAc/H₂O (4:4:0.7) | | NaHCO₃ (2.5 eq.) | | | | | |
| 53 | (B) RuCl₃*H₂O 0.55% eq | B | Oxone (2.6 eq.) | 10′ | 35 | — | 9 | |
| | EtOAc/H₂O (2.5:6) | | | | | | | |
| 54 | RuCl₃*H₂O 3.5% eq | A | Oxone (1.5 eq.) | 10′ | 52 | 10 | 12 | C |
| | ACN/H₂O (7.5:4.5) | | NaHCO₃ (4.6 eq.) | | | | | |
| 55 | RuCl₃*H₂O 3.5% eq | A | Oxone (1.5 eq.) | 10′ | 43 | 21 | 2 | add Na₂S₂O₃ |
| | ACN/H₂O (2:1) | | NaHCO₃ (4.6 eq.) | | | | | and concentrate |
| 56 | RuCl₃*H₂O 3.5% eq | A | Oxone (3 eq.) | 10′ | 37 | 15 | <1 | add Na₂S₂O₃ |
| | ACN/H₂O (7.5:4.5) | | NaHCO₃ (9 eq.) | | | | | and concentrate |

TABLE 30
Results of Synthesis Examples 57 to 66: Synthesis of (2) and (3)
with RuCl₃•H₂O and T-HYDRO (tert-Butyl hydroperoxide, 70% in H₂O)

| | | Catalyst | | | | | | |
|---|---|---|---|---|---|---|---|---|
| Synthesis | | Preparation | | | GC results (ala) | | | |
| Example | [Ru Precatalyst] | Type | [Ox] | t | SM | INT | FP | Work up |
| 57 | (B) RuCl₃•H₂O 1.0% eq | B | T-HYDRO (5 eq.) | 1 h | 57 | 12 | 2* | B |
| | ACN/EtOAc/H₂O (6:6:1) | | | | | | | |
| | TBAOH (1.5 eq) | | | | | | | |
| 58 | (B) RuCl₃•H₂O 0.55% eq | B | T-HYDRO (2.6 eq.) | 10′ | 51 | 27 | 10 | B |
| | EtOAc/H₂O (2.5:6) | | | | | | | |
| 59 | (B) RuCl₃•H₂O 0.55% eq | B | T-HYDRO (2.6 eq.) | 4 h | 64 | 31 | <1 | D |
| | EtOAc/H₂O (2.5:6) | | | | | | | |
| 60 | (B) RuCl₃•H₂O 0.55% eq | B | T-HYDRO (2.6 eq.) | 4 h | 49 | 40 | 1 | D |
| | EtOAc/H₂O (2.5:6) | | | 50° C. | | | | |
| 61 | (B) RuCl₃•H₂O 0.55% eq | B | T-HYDRO (2.6 eq.) | 24 h | 45 | 49 | 1 | D |
| | EtOAc/H₂O (2.5:6) | | | | | | | |
| 62 | (B) RuCl₃•H₂O 0.55% eq | B | T-HYDRO (2.6 eq.) | 24 h | 49 | 35 | 3 | add/-PrOH and |
| | EtOAc/H₂O (2.5:6) | | | 50° C. | | | | concentrate |
| 63 | (B) RuCl₃•H₂O 3.5% eq | B | T-HYDRO (2.6 eq.) | 24 h | 61 | 22 | 10 | add/-PrOH and |
| | EtOAc/H₂O (2.5:6) | | | 50° C. | | | | concentrate |
| 64 | (B) RuCl₃•H₂O 0.55% eq | B | T-HYDRO (2.6 eq.) | 10′ | 51 | 27 | 10 | B |
| | EtOAc/H₂O (2.5:6) | | | | | | | |
| 65 | (B) RuCl₃•H₂O 0.55% eq | B | T-HYDRO (5 eq.) | 24 h | 41 | 37 | 3 | add/-PrOH and |
| | EtOAc/H₂O (2.5:6) | | | | | | | concentrate |
| 66 | (B) RuCl₃•H₂O 0.55% eq | B | T-HYDRO (5 eq.) | 24 h | 59 | 33 | <1 | add/-PrOH and |
| | ACN/EtOAc/H₂O (6:6:1) | | | | | | | concentrate |

TBAOH = tert.-Butylammoniumhydroxide
T-HYDRO = te/T-Butylammonium hydroperoxide

TABLE 31
Results of Synthesis Examples 67 to 76: Synthesis of (2) and (3) with RuCl₃•H₂O or RuO₂•H₂O and KBrO₃

| | | Cat. | | | | | | |
|---|---|---|---|---|---|---|---|---|
| Synthesis | | Preparation | | | GC results (a/a) | | | |
| Example | [Precatalyst] | Type | [Ox] | t | SM | INT | FP | Work up |
| 67 | RuCl₃*H₂O 0.55% eq | B | KBrO₃ (2.6 eq.) | 4 h | 65 | 30 | <1 | |
| | EtOAc/H₂O (2.5:6) | | | 50° C. | | | | |
| 68 | RuCl₃*H₂O 0.55% eq | B | KBrO₃ (2.6 eq.) | 10′ | 87 | 6 | <1 | add iPrOH and |
| | EtOAc/H₂O (2.5:1.25) | | | | | | | concentrate |
| 69 | RuCl₃*H₂O 0.55% eq | B | KBrO₃ (5.0 eq.) | 10′ | 63 | 9 | <1 | add iPrOH and |
| | EtOAc/H₂O (2.5:6) | | | | | | | concentrate |
| 70 | RuCl₃*H₂O 5.0% eq | B | KBrO₃ (2.6 eq.) | 10′ | 41 | 29 | 10 | add iPrOH and |
| | EtOAc/H₂O (2.5:6) | | | | | | | concentrate |
| 71 | RuCl₃*H₂O 5.0% + 5.0% eq | B | KBrO₃ (2.6 eq.) | 10′ + 10′ | 12 | 10 | 38 | add iPrOH and |
| | EtOAc/H₂O (2.5:6) | | | | | | | concentrate |
| 72 | RuCl₃*H₂O 5.0% + 5.0% eq | B | KBrO₃ (2.6 eq.) | 10′ + 30′ | 10 | 16 | 8 | add iPrOH and |
| | EtOAc/H₂O (2.5:6) | | | | | | | concentrate |
| 73 | RuCl₃*H₂O 0.55% + 0.55% eq | B | KBrO₃ (2.6 eq.) | 10′ + 30′ | 49 | 6 | 0 | add iPrOH and |
| | EtOAc/H₂O (2.5:6) | | | | | | | concentrate |
| 74 | RuO₂*H₂O 5.0% + 5.0% eq | B | KBrO₃ (2.6 eq.) | 10′ + 10′ | 54 | 27 | 4 | add iPrOH and |
| | EtOAc/H₂O (2.5:6) | | | | | | | concentrate |
| 75 | RuCl₃*H₂O 5.0% + 5.0% eq | B | KBrO₃ (2.6 eq.) | 10′ + 10′ | 17 | 21 | 18 | add iPrOH and |
| | EtOAc/H₂O (2.5:6) | | | 50° C. | | | | concentrate |
| 76 | RuCl₃*H₂O 3.5% eq | B | KBrO₃ (2.6 eq.) | 4 h | 13 | 58 | 6 | add iPrOH and |
| | EtOAc/H₂O (2.5:6) | | | 50° C. | | | | concentrate |

TABLE 32
Results of Synthesis Examples 77 to 79: Synthesis of (2) and (3) with RuCl₃•H₂O and further oxidants

| | | Cat. | | | | | | |
|---|---|---|---|---|---|---|---|---|
| Synthesis | | Preparation | | | GC results (a/a) | | | |
| Example | [Precatalyst] | Type | [Ox] | t | SM | INT | FP | Work up |
| 77 | (B) RuCl₃*H₂O 0.55% eq | B | HIO₄ (2.6 eq.) | 10′ | 23 | — | 31 | B |
| | EtOAc/H₂O (2.5:6) | | | | | | | |
| 78 | (B) RuCl₃*H₂O 0.55% eq | B | DIB (2.6 eq.) | 10′ | 72 | — | 4 | add/-PrOH and |
| | EtOAc/H₂O (2.5:6) | | | | | | | concentrate |
| 79 | (B) RuCl₃*H₂O 0.55% eq | B | DIB (2.6 eq.) | overnight | 56 | 1 | 6 | add/-PrOH and |
| | EtOAc/H₂O (2.5:6) | | | | | | | concentrate |

TABLE 33
Results of Synthesis Examples 80 to 82: Synthesis of
intermediate (2) and Final Product (3) with Iodine

| Synthesis | | | | GC results (a/a) | | | |
|---|---|---|---|---|---|---|---|
| Example | Catalyst | Solvent | t | SM | INT | FP | Work up |
| 80 | I₂ (3 eq) | H₂O/THF (1:2.5) | 30′ | 37 | 41 | 6 | add Na₂S₂O₃ and |
| | NaHCO₃ (10 eq.) | | | | | | aq. extraction |
| 81 | I₂ (3 eq) | H₂O/THF (1:2.5) | 1 h | 9 | 11 | 3 | add Na₂S₂O₃ and |
| | NaHCO₃ (5 eq.) | | | | | | concentrate |
| 82 | I₂ (3 eq) | H₂O/THF (1:2.5) | 1 h | 9 | 6 | 0 | add Na₂S₂O₃ and |
| | NaHCO₃ (5 eq.) | | | | | | concentrate |

TABLE 34
Results of Synthesis Examples 83 to 86: Synthesis of (2) with Fe-based catalysts

| Synthesis | | | | GC results (a/a) | | |
|---|---|---|---|---|---|---|
| Example | [Precatalyst] | [Ox] | t | SM | INT | FP |
| 83 | FeCl₃ (0.2 eq) | T-HYDRO (2.2 eq) | 1 h | 22 | 72 | — |
| | H₂O (1 mL) | | 50° C. | | | |
| 84 | FeSO₄ (0.2 eq) | T-HYDRO (2.2 eq) | 24 h | 23 | 59 | — |
| | H₂O (1 mL) | | 50° C. | | | |
| 85 | FeCl₃ (0.2 eq) | T-HYDRO (2.2 eq) | 24 h | 38 | 16 | — |
| | H₂O (1 mL) | | 70° C. | | | |
| 86 | FeCl₃ (0.2 eq) | T-HYDRO (2.2 eq) | 72 h | 37 | 19 | — |
| | H₂O (1 mL) | | 50° C. | | | |

### Experimental Section—Part A (Continued)

### Screening Example 22: Synthesis of (S)-2-(2-oxopyrrolidin-1-yl)butane amide (3) with RuO₂*xH₂O/NaIO₄ in different aqueous/organic solvent systems and scale-up

The reaction was performed according to RP 1 under the conditions as specified below in Table 35. The observed results are also summarized in Table 35.

As can be seen, a biphasic solvent system with ethyl acetate/water resulted in the production of up to 76% of the desired product even at room temperature and at low amount of organic solvent, which is favorable from a technical point of view. It may be assumed that RuO₄ is transferred into the organic layer where, due to the low water content, the intermediate 2 might be protected from side reactions, like ring-opening and/or polymerization. The more polar product 3 in turn is transferred to the aqueous phase, where it might be protected from overoxidation.

The reaction was scaled up ten-fold obtaining a reproducible yield of 74% (table 35, entry 8, 1.0 g of (S)-1)) After the reaction, RuO₂ was efficiently recovered by filtration over aluminum oxide and the sodium iodate was recovered quantitatively by crystallization with methanol (up to 95% isolated yield).

TABLE 35
Screening of different aqueous/organic solvent systems

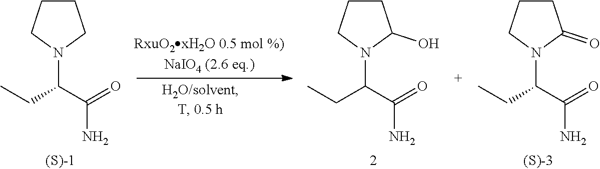

| | | | | GC Yield [%] | | | |
|---|---|---|---|---|---|---|---|
| Entry | Solvent | [v %] | T [° C.] | 1 | 2^{a} | 3 | □ |
| 1 | H₂O | — | 20 | 5 | 9 | 31 | 45 |
| 2 | DMF | 33 | 20 | 16 | 18 | 37 | 72 |
| 3 | MeCN | 33 | 20 | 1 | 4 | 57 | 62 |
| 4 | acetone | 33 | 20 | 1 | 4 | 60 | 65 |
| 5 | MeCN | 50 | 0 | 1 | 2 | 65 | 68 |
| 6 | MeCN | 70 | 0 | 0 | 0 | 66 | 66 |
| 7 | EtOAc | 33 | 20 | — | — | 76 | isol. |
| 8 | EtOAc | 33 | 20 | — | — | 74 | isol. |

^{a}relative intensity vs. standard, eq. = equivalents, T = temperature, DMF = dimethylformamide, isol. = isolated yield. Screenings were performed on a 100 mg scale of (S)-1.

### Screening Example 23: Electrochemical Oxidation of Sodium Iodate

Sodium iodate (0.21 M) and sodium hydroxide (1.00 M) were dissolved in water and were electrolyzed in a flow electrolysis cell. The electrolysis cell was equipped with a BDD anode and a stainless steel cathode and was divided by a Nafion membrane. The anolyte and catholyte were pumped in two independent cycles in cascade-mode. Two Ritmo R033 pumps from Fink Chem+Tec GmbH (Leinfelden-Echterdingen, Germany) were used.

Selected results are shown in Table 36. The electrolysis conditions were adjusted to an increased current density of j=100 mA/cm² (Table 36, entries 4-7) and an applied charge of Q=4 F (Table 36, entries 8-10). The product was obtained in 78% yield, which corresponds to 48 g of paraperiodate.

TABLE 36
Scale up of the iodate oxidation

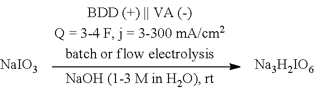

| | | | | | LC-PDA Yield [%] | | |
|---|---|---|---|---|---|---|---|
| Entry | V [mL] | Q [F] | j [mA/cm²] | C(NaOH) [M] | IO₃⁻ | IO₄⁻ | Σ |
| 1 | 6 | 3 | 2 | 3 | 20 | 83 | 103 |
| 2 | 6 | 3 | 10 | 1 | 16 | 83 | 99 |
| 3 | 6 | 3 | 10 | 1 | 11 | 86 | 97 |
| 4 | 50 | 3 | 10 | 1 | 20 | 85 | 105 |
| 5 | 50 | 3 | 50 | 1 | 26 | 73 | 99 |
| 6 | 50 | 3 | 100 | 1 | 34 | 63 | 97 |
| 7 | 50 | 3 | 300 | 1 | 63 | 37 | 100 |
| 8 | 500 | 3 | 100 | 1 | 29 | 70 | 99 |
| 9 | 1000 | 3 | 100 | 1 | 37 | 72 | 109 |
| 10 | 1000 | 4 | 100 | 1 | 18 | 78 | 96 |

BBD = boron-doped diamond, VA = stainless steel, rt = room temperature. Reactions were performed with an initial concentration of iodate of 0.21 M.

### Synthesis Example 87: Synthesis of (S)-2-(2-oxopyrrolidin-1-yl)Butane Amide (3) with RuO₂*xH₂O/NaIO₄ and Calculation of E-factor (EF) and Atom Efficiency (AE)

The reaction was performed according to RP 1 under the conditions as specified below in Table 37 and EF and AE parameters were determined according to the following formulae:

$$A E = \frac{M ( desired product )}{M ( all products )}$$

$$EF = \frac{m ( raw materials ) - m ( desired product )}{m ( desired product )}$$

TABLE 37
AE and EF Values

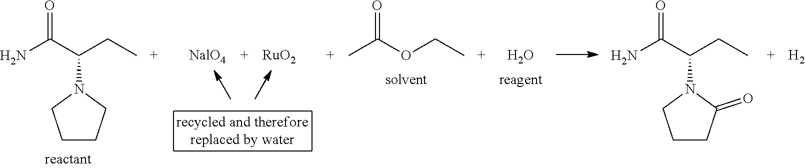

| Reactants | | | | Products | |
|---|---|---|---|---|---|
| Formula | C₈H₁₆N₂O | C₄H₈O₂ | H₂O | Formula | C₈H₁₄N₂O₂ |
| MW | 156.23 | 88.11 | 18.02 | MW | 170.21 |
| Limiting? | Yes | No | No | Equivalents | 76.00% |
| Equivalents | | 21.27 | 2.60 | % Completion | 82.80 mg |
| Sample Mass | | | 29.98 mg | Expected Mass | 486.47 μmol |
| % Weight | | | | Expected Moles | |
| Molarity | | | | Measured Mass | |
| Density | | 902.00 mg/mL | | Purity | |
| Volume | | 1.33 mL | | Product Mass | |
| Reactant Moles | 640.09 mol | 13.62 mmoL | 1.66 mmol | Product Moles | |
| Reactant Mass | 100.00 mg | 1.20 g | 29.98 mg | % Yield | |

AE = 170.21: (170.21 + 2.02) = 99%
EF = (100 + 1200 + 29.98 − 82.8): 82.8 = 15.06 [mg/mg]

### Screening Example 23: Development of Flow Electrolysis and Scale-Up

Sodium iodate (0.21 M) and sodium hydroxide (1.00 M) were dissolved in water and were electrolysed in a flow electrolysis cell. The electrolysis cell was equipped with a BDD anode and a stainless-steel cathode and was divided by a Nafion membrane. The anolyte and catholyte were pumped in two independent loops in cycling-mode. Two Ritmo R033 pumps from Fink Chem+Tec GmbH (Leinfelden-Echterdingen, Germany) were used. The results are summarized in Table 37

TABLE 37
Flow cell electrolysis of sodium iodate

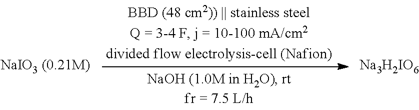

| | | | | LC-PDA | | |
|---|---|---|---|---|---|---|
| | | | | Yield [%] | | |
| Entry | V [mL] | j [mA/cm²] | Q [F] | IO₃_{^{−}} | IO₄_{^{−}} | Σ |
| 1 | 50 | 10 | 3 | 9 | 83 | 92 |
| 2 | 50 | 10 | 3 | 20 | 85 | 105 |
| 3 | 50 | 50 | 3 | 26 | 73 | 99 |
| 4 | 50 | 50 | 4 | 16 | 76 | 92 |
| 5 | 50 | 100 | 3 | 34 | 63 | 97 |
| 6 | 50 | 100 | 4 | 24 | 72 | 96 |
| 7 | 50 | 300 | 3 | 63 | 37 | 100 |
| 8 | 50 | 300 | 4 | 44 | 53 | 97 |
| 9 | 500 | 100 | 3 | 29 | 70 | 99 |
| 10 | 500 | 100 | 4 | 14 | 88 | 102 |
| 11 | 500 | 300 | 3 | 43 | 56 | 99 |
| 12 | 500 | 300 | 4 | 32 | 70 | 102 |
| 13 | 1000 | 100 | 3 | 37 | 72 | 109 |
| 14 | 1000 | 100 | 4 | 18 | 78 | 96 |

* starting material did not dissolve completely before reaction, dissolved and new solid formed during the reaction.

### Synthesis Example 88: Recrystallization of Paraperiodate to Metaperiodate

Sodium paraperiodate (4.00 g, 13.6 mmol), HNO₃ (2.2 mL, 65%) and water (8 mL) were refluxed at 130° C. for several minutes. Water was distilled off until crystallisation started. The mixture was cooled to 4° C. and was kept at this temperature overnight. The crystals were filtered off and were dried under vacuum. Sodium metaperiodate was obtained as colourless crystals (2.057 g, 9.62 mmol, 71%). IR data were in accordance with the Bio-Rad database (Infrared spectral data were obtained from the Bio-Rad/Sadtler IR Data Collection, Bio-Rad Laboratories, Philadelphia, Pa. (US) and can be found under https://spectrabase.com. Spectrum ID (meta-periodate): 3ZPsHGmepSu).

### Synthesis Example 89: Electrolysis with Recovered Sodium Iodate

Sodium iodate (2.08 g, 10.5 mmol) recovered from the ruthenium-catalysed step in levetiracetam synthesis by precipitation through addition of alcohol (methanol or iso-propanol) and sodium hydroxide (2.00 g, 50.0 mmol) were dissolved in water (50 mL) and were electrolysed according to RP 3. A current density of j=50 mA cm⁻² and a charge amount of Q=4 F (4055 C) were applied. Sodium paraperiodate was obtained in reproducible 83% yield as determined by LC-PDA.

1. A process for the preparation of an oxidized heterocyclic alpha-amino amide compound of the general formula II 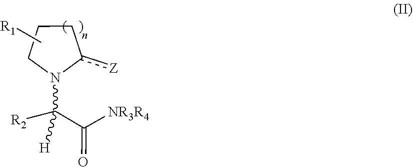
wherein
the group Z is bound to the heterocyclic ring via a single or a double bond and is selected from —OH and ═O;
n is 0 or an integer of 1 to 4;
R₁ and R₂ independently of each other represent H or a straight chain or branched, saturated or non-saturated hydrocarbon group having 1 to 6 carbon atoms;
R₃ and R₄ independently of each other represent H, a straight-chain or branched, saturated or non-saturated hydrocarbon group having 1 to 6 carbon atoms; or form, together with the nitrogen atom to which they are bound, a saturated or non-saturated, non-aromatic or aromatic, heterocyclic 4- to 7-membered ring group carrying one or more ring heteroatoms;
which process comprises
1) either contacting a non-oxidized heterocyclic alpha-amino amide compound of the general formula I 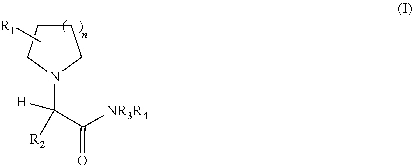
wherein Z, n and R₁ to R₄ have the same meanings as defined above,
with an oxidation catalyst thereby oxidizing the heterocyclic alpha-amino group in a compound of formula I, in particular thereby introducing a keto- or hydroxyl group into the heterocyclic amino residue at its alpha-methylene group of a compound of formula I,
or
performing an electrochemical (anodic) oxidation of a compound of formula I as defined above;
and
2) optionally isolating a compound of formula II.

2. The process of claim 1, wherein the oxidation catalyst is selected from:
a) an optionally immobilized inorganic ruthenium salt, in particular a ruthenium (+III), (+IV), (+V), or (+VI) salt, more particularly a ruthenium (+III) or (+IV) salt, and at least one oxidant capable of in situ oxidizing the ruthenium cation, in particular the ruthenium (+III), (+IV), (+V), or (+VI) cation, more particularly the ruthenium (+III) or (+IV) cation, in particular to ruthenium (+VIII), and optionally in the presence of a mono- or polyvalent metal binding ligand, as for example sodium oxalate (ox) or acetylacetonate (acac);
b) an inorganic iron (+II) or (+III) salt and at least one oxidant capable of in situ oxidizing the iron (+II) or (+III) cation, and optionally in the presence of a mono- or polyvalent metal ligand, as for example sodium oxalate oxalate (ox) or acetylacetonate (acac)
c) I₂/NaHCO₃
d) Au-coated Al₂O₃; and
e) combinations thereof.

3. The process of claim 2, wherein the inorganic ruthenium (+III) or (+IV) salt is selected from RuCl₃, RuO₂ and the respective hydrates, in particular monohydrates, thereof, and wherein the oxidant is selected from
a) alkali perhalogenates, and the hydrates thereof
b) alkali hypohalogenites, and the hydrates thereof
c) Oxone (MPS, potassium monopersulfate)
d) tert-butyl hydroperoxide (T-HYDRO), optionally in combination with KOtBu
e) HIO₄
f) KBrO₃
g) hypervalent iodide compounds, e.g. DIB (diacetoxyiodobenzene), and
h) combinations thereof.

4. The process of claim 2, wherein the, wherein the inorganic iron (+II) or (+III) salt is selected from FeCl₂, FeCl₃, FeSO₄ and the respective hydrates, and wherein the oxidant is selected from
a) hydrogen peroxide
b) T-HYDRO
c) PhCO₃tBu and
d) combinations thereof.

5. The process of claim 3, wherein the oxidant is selected from
a) alkali metal periodates, wherein the periodate is a para-periodate, meta-periodate, ortho-periodate or a mixture of two or three of these periodates, and is in particular a para-periodate, a meta-periodate or a mixture of a para-periodate and a meta-periodate, the double salts of such alkali metal periodates, e.g. NaIO₄*2NaOH; and wherein the alkali metal is in particular sodium;
b) alkali metal hypohalogenites, in particular alkali metal hypochlorites, more particularly NaOCl, and the hydrates thereof, in particular NaOCl*5H₂O;
c) mixtures of a) and b); or
d) mixtures of a) and at least one co-oxidant selected from hydrogen peroxide; Oxone (MPS, potassium monopersulfate); T-HYDRO, optionally in combination with KOtBu; I₂/NaHCO₃; HIO₄, KBrO₃ and hypervalent iodide compounds, e.g. DIB (diacetoxyiodobenzene).

6. The process of claim 1 wherein the reaction product comprises as oxidation product a compound of formula II or a mixture of at least two compounds of formula II.

7. The process of claim 6 wherein the reaction product comprises a compound of the formula III or IV 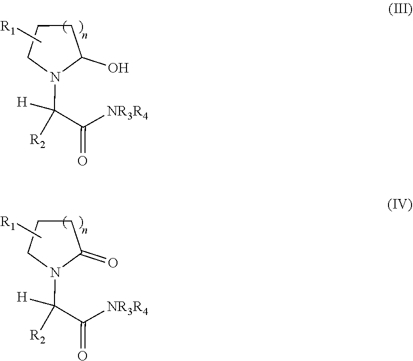
wherein n and R₁ to R₄ have the same meanings as defined above,
or a mixture of at least two of said compounds.

8. The process of claim 1, wherein the reaction product comprises a compound of the formula III or IV in stereoisomerically essentially pure or enriched form, or as a mixture of at least two stereoisomers.

9. The process of claim 1, wherein said non-oxidized heterocyclic alpha-amino amide compound of the general formula I is applied as a mixture of stereoisomers or in stereoisomerically essentially pure or enriched form.

10. The process of claim 9, wherein the oxidation reaction is performed under essential retention of the stereochemical configuration, in particular at the alpha carbon atom.

11. The process of claim 1, wherein the reaction product comprises one of the following stereoisomer of a compound of formula IV 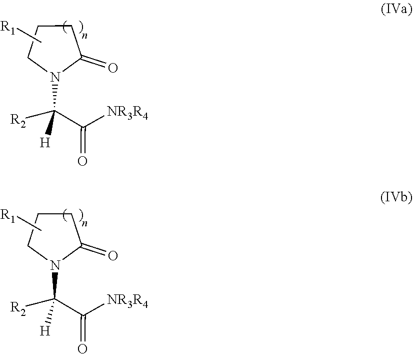
wherein n and R₁ to R₄ have the same meanings as defined above,
or a mixture thereof.

12. The process of claim 11, wherein the reaction product comprises a stereoisomer of formula LIVa or LIVb 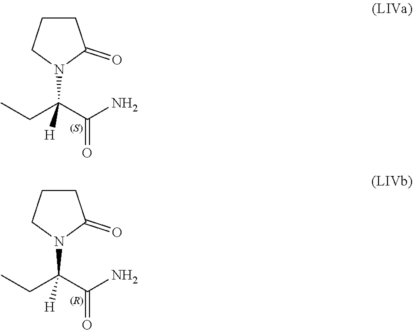
or a mixture thereof.

13. The process of claim 11, wherein the reaction product comprises an stereoisomer of formula LVIa or LVIb 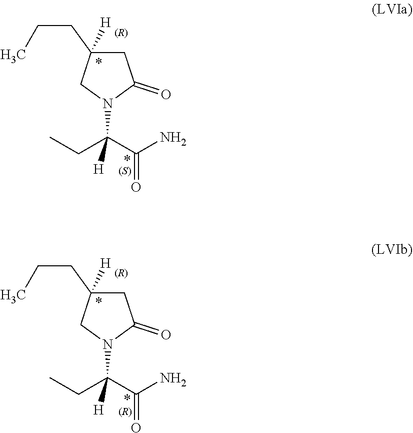
or a mixture of stereoisomers (diastereoisomers) thereof.

14. The process of claim 1 wherein the reaction product comprises a compound of formula LV

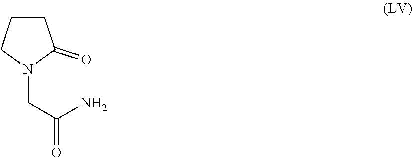

15. The process of claim 1, wherein said oxidation is performed by reacting an aqueous or aqueous-organic solution of said compound of formula I at a temperature in the range of 0 to 30° C. with the oxidation catalyst.

16. The process of claim 2, wherein the oxidation is performed by reacting said compound of formula I with a catalytic amount of said inorganic ruthenium (+III) or (+IV) salt and the oxidant, wherein the initial molar ratio of compound of formula I′ and oxidant is in the range of 1:1 to 1:5, in particular 1:1.5 to 1:3.

17. The process of claim 2, wherein the mono- or polyvalent metal ligand is added to the reaction mixture, so that the molar ratio of ruthenium (+III) or (+IV) salt to ligand is in the range of 1:1 to 1:5, in particular 1:1.5 to 1:2.5.

18. The use of a catalyst or catalyst combination as defined in claim 2 in a method for oxidation of heterocyclic alpha-amino amide compounds, carrying a cyclic amino substituent at an asymmetric carbon atom in alpha-position the carbonyl C-atom of the amide group.

19. The process of claim 1, wherein the electrochemical oxidation of a compound of formula I is performed under at least one of the following conditions:
a) anode material selected from glass-like carbon (GLC), graphite, boron-doped diamond (BDD), carbon fiber, Ni, Pt, Cu, and Au, and in particular Pt, GLC or BDD,
b) aqueous solution of at least one compound of formula I′ at an initial concentration of 0.001 to 0.1 M,
c) pH of the aqueous solution of 7 or more, in particular 8 or more,
d) temperature in the range of 0 to 80° C.,
e) voltage in the range 1 to 30 V,
f) current density in the range of 0.5 to 500 mA/cm²; and
g) applied charge in the range of 1 to 10 Farad,
in particular a combination comprising at least features a), b), c), f) and g).

20. The process of claim 3, further comprising the recovering and electrochemical recycling of the spent alkali perhalogenate oxidant, in particular of the electrochemical oxidation of an alkali halogenate back to an alkali perhalogenate oxidant.

21. The process of claim 20, wherein the electrochemical recycling comprises an anodic oxidation of an alkali halogenate back to an alkali perhalogenate oxidant.

22. The process of claim 20, wherein a boron-doped diamond anode is applied.

23. The method of claim 20, wherein the oxidation is performed under at least one of the following conditions:
a) aqueous solution of at least one alkali halogenate at an initial concentration of 0.001 to 10 M,
b) pH of the aqueous solution of 7 or more,
c) temperature in the range of 0 to 80° C.,
d) voltage in the range of 1 to 30V,
e) current density in the range of 10 to 500 mA/cm²; and
f) applied charge in the range of 1 to 10 Farad,
in particular a combination comprising at least features a), b), e) and f).

24. A process for the preparation of sodium para-periodate, which process comprises the electrochemical anodic oxidation of an aqueous solution of sodium iodate to sodium para-periodate under basic conditions so that sodium para-periodate precipitates in the anolyte, wherein a carbon-comprising anode, in particular a boron-doped diamond anode is applied.

25. A process for the preparation of sodium para-periodate, which process comprises the electrochemical anodic oxidation of an aqueous solution of sodium iodate to sodium pa-ra-periodate under basic conditions so that sodium para-periodate precipitates in the anolyte, wherein a carbon-comprising anode, in particular a boron-doped diamond anode is applied, wherein the anodic oxidation is performed under a combination of the conditions a), b), e) and f) and optionally at least one of c) and d) as defined below:
a) aqueous solution of at least one sodium iodate at an initial concentration cₒ of 0.001 to 1 M,
b) pH of the aqueous solution of 12 or more,
c) temperature in the range of 0 to 80° C.,
d) voltage in the range of 1 to 30V,
e) current density in the range of 10 to 500 mA/cm²; and
f) applied charge in the range of 1 to 10 Farad.

26. The process of claim 24, wherein the aqueous solution of sodium iodate contains NaOH at an initial molarity in the range of 0.6 to 3 M, in particular 0.9 to 2 M and specifically 1 M.

27. The process of claim 24, wherein the ratio of base to iodate is from 8:1 to 2:1, more particularly 6:1 to 3:1, and specifically in the range of 5:1 to 4:1.

28. The process of claim 1, wherein the anodic oxidation is performed under a combination of the following conditions:
initial concentration cₒ (NaIO3) of 0.21 M ±25%, in particular ±15%, ±10%, more particularly ±5%, ±2% or ±1%
initial concentration cₒ (NaOH) of 1.0 M ±25%, in particular ±15%, ±10%, more particularly ±5%, ±2% or ±1%
ratio of cₒ (NaIO₃):c₀ (NaOH) of about 1:5±25%, in particular ±15%, ±10%, more particularly ±5%, ±2% or ±1%

29. The process of claim 28, wherein the anodic oxidation is performed under a combination of the following conditions:
current density j in the range of 50 to 100 mA/cm² in batch electrolysis; or current density j in the range of 400 to 500 mA/cm² in flow electrolysis
applied charge Q in the range of 3 to 4 F
initial concentration cₒ (NaIO3) of 0.21 M ±25%, in particular ±15%, ±10%, more particularly ±5%, ±2% or ±1%
initial concentration cₒ (NaOH) of 1.0 M ±25%, in particular ±15%, ±10%, more particularly ±5%, ±2% or ±1%
ratio of cₒ (NaIO3):cₒ (NaOH) of 1:5 ±25%, in particular ±15%, ±10%, more particularly ±5%, ±2% or ±1%

30. The process for the preparation of sodium para-periodate of claim 24, wherein.
a(1) the aqueous solution said sodium iodate is applied at an initial concentration c₀ of 0.001 to 5 M or 0.001 to 1 M, more preferably from 0.001 to 2 M, in particular from 0.01 to 1 M or 0.01 to 0.5 M or 0.01 to 0.4 M or 0.05 to 0.25 M, and specifically from 0.1 to 0.3 M or 0.1 to 0. 25 M;
b(2) the initial molarity cₒ of the base is in the range of 0.3 to 5 M, preferably 0.6 to 3 M, in particular 0.9 to 2 M and specifically 1 M; and
c(3) the ratio of c₀ (base) to cₒ (iodate) is in the range of 10:1 to 1:1, more particularly from 8:1 to 2:1, even more particular 6:1 to 3:1, and specifically 5:1 to 4:1, wherein the base is NaOH.

31. The process of claim 24, wherein the anodic oxidation is per-formed in an electrolytic flow cell.

32. The process of claim 24, wherein the sodium para-periodate is isolated by filtration of the precipitate as contained in the anolyte.

33. The process of claim 32, wherein the sodium para-periodate is neutralized be the addition of acid in order to obtain sodium meta-periodate.
